(19) United States
(12) Patent Application Publication
(10) Pub. No.: US 2022/0205054 A1
(43) Pub. Date: Jun. 30, 2022

(54) QUANTITATIVE PROTEIN ANALYSIS

(71) Applicant: SpectraFlora Group Pty Ltd, Kirribilli (AU)

(72) Inventor: Steve Van Sluyter, Sydney (AU)

(21) Appl. No.: 17/554,980

(22) Filed: Dec. 17, 2021

(30) Foreign Application Priority Data
Dec. 18, 2020 (AU) 2020904736

(51) Int. Cl. C12Q 1/6895

(52) U.S. Cl. CPC C12Q 1/6895

(57) ABSTRACT

The disclosure relates to quantitative analysis of proteins in different species, including plant species. Disclosed are methods that utilize conserved peptides across species to be used as isotope labeled internal standards, which are then used for absolute quantification of proteins. For example, a method for quantitative protein analysis of two or more species is disclosed, the method including determining a set of common peptides that are common for the two or more species, creating a set of isotope-labeled peptides out of the set of common peptides, adding a predefined amount of the labeled peptides to a sample from one of the two or more species, performing mass spectrometry to create first intensity values for a group of peptides from the sample and second intensity values for the labeled peptides, and calculating a quantitative amount of the group of peptides based on the first intensity values and the second intensity values.

### CROSS-REFERENCE TO RELATED APPLICATIONS

This application claims priority to Australian Patent Application No. 2020904736, filed Dec. 18, 2020, which is hereby incorporated by reference in its entirety.

### REFERENCE TO SEQUENCE LISTING

This application contains a Sequence Listing that has been submitted electronically in ASCII format and is hereby incorporated by reference in its entirety. The Sequence Listing was created on Mar. 8, 2022, has a file name of 17554980_ST25.txt, and is 112 kilobytes in size.

### FIELD OF THE INVENTION

This disclosure relates to quantitative analysis of proteins across different species, including various species of plants.

### BACKGROUND

The vast majority of quantitative proteomics experiments use relative quantification that assigns unitless values as measures of protein amounts that are only meaningful among limited comparisons; specifically, comparisons of the same protein across treatments within an experiment. It is not possible with relative quantification results to make quantitative comparisons across different proteins, different species, or different experiments. Despite those limitations, relative quantification is widely used because it is less expensive and easier to implement than absolute quantification.

Absolute quantification makes it possible to measure proteins in real units, for example moles or grams of a protein per cell, per dry weight of tissue, per leaf area, per total protein in a sample, per absolute amount of another protein in the sample, etc. Real units of measurement enable quantitative comparisons of protein amounts across different proteins, different species, different experiments, and different laboratories.

Absolute quantification uses isotope labeled internal peptide standards, which are carefully selected, manufactured, purified, quantified, and spiked into experimental samples prior to mass spectrometry. Typically, unique peptides—peptides that only appear in a single isoform of a protein—are selected as internal standards so that non-target proteins do not interfere with the quantitative results. Some analysis software contains features that automatically exclude signals from peptides that are not unique. The limitation of using unique peptides is that they are specific to a single species. Consequently, most isotopically labeled internal peptide standards in quantitative proteomics experiments can only be used with a single species, making it time consuming and expensive to conduct absolute quantification experiments with multiple species—each new species requires a new set of internal peptide standards.

Given the foregoing, needs exist for novel methods, devices, and systems for quantitative analysis of proteins in different species, including plant species.

### SUMMARY

It is to be understood that both the following summary and the detailed description are exemplary and explanatory and are intended to provide further explanation of the invention as claimed. Neither the summary nor the description that follows is intended to define or limit the scope of the invention to the particular features mentioned in the summary or in the description.

In general, the present disclosure is directed towards novel methods, devices, and systems for quantitative analysis of proteins in different species, including plant species.

Protein quantities are an important factor in the assessment of a sample from a species. For example, the amount of a protein in plant matter can be a valuable indicator about the plant's qualities. As such, the observation of proteins in a plant can be considered a molecular phenotype of that plant. Accordingly, this protein phenotype can be used for selective breeding. For example, consider heat shock protein A (HSPA) that is highly expressed in response to acute subcellular heat damage. If HSPA amounts are higher in species X than Y under identical heat wave conditions, and macroscopic physiology does not change for either species, then species Y must possess an additional mechanism to cope with heat stress.

The example above relies on a quantitative assessment of plant proteins, that is, it relies on measuring the quantitative amount of a protein in the plant. However, quantitative assessments of proteins are generally difficult to perform in an accurate manner. This problem occurs because ultimately, current protein detection methods, such as mass spectrometry, split the proteins into peptides and only detect fragments of the peptides. However, each fragment behaves differently from a quantitative point of view and therefore, mass spectrometers perform peak detection to identify fragments, which does not enable a quantitative assessment. In other words, the height or amplitude of each peak does not provide an accurate measure of the quantity of the protein.

FIG. 1 illustrates a mass spectrometer 100 for analyzing a protein 101. Protein 101 is part of a plant sample, such as a leaf tissue. However, intact proteins in complex samples create signals that are too complex to readily interpret. Therefore, protein 101 is digested 102 by a protease (such as Trypsin) into peptides. The peptides are fed into a liquid chromatography (LC) column 103, from which the peptides elute into a quadrupole 104 followed by a collision cell 105 and a time of flight analyzer 106 comprising a grouping chamber 107, accelerator 108, and a detector 109.

When in use, the digestion 102 essentially “cuts” the protein 101 into peptides at predictable locations due to the chemical structure of the protein. For ease of presentation, the peptides are represented as circles in FIG. 1. The LC column 103 separates the peptides based on how long they take to pass through the column 103, which is referred to herein as “retention time.” This ensures that at any one point in time only a small number of different peptides elute from LC column 103, which greatly simplifies protein identification downstream. It is important to note that the retention time is typically independent from the mass-to-charge ratio (noting that the peptides are charged at this point). In other words, the peptides eluting from the LC column at any point in time, could have a m/z ratio distribution across the entire range of the spectrometer 100. The peptides entering the quadrupole 104 are also referred to as “precursor peptides” or “precursor ions.”

In a first measurement (also referred to herein as “first scan,” or MS1), the peptides are ionized and quadrupole 104 deactivated (precursor isolation window opened wide). The collision cell 105 is also turned off so that all peptides pass through to the TOF analyzer 106 and are detected across their m/z range.

In a second measurement (also referred to herein as “second scan,” or MS2), the quadrupole 104 is activated by applying a varying electromagnetic field onto four rod-shaped electrodes. Upon entry into the quadrupole 104, the peptides are charged and due to their different mass-to-charge ratio (m/z), they are affected differently by the electric field generated by the electrodes. As a result, only peptides in a specific range of m/z ratio exit the quadrupole 104. The other peptides are blocked and/or absorbed. This m/z range is also referred to as a precursor selection window or simply selection window. The selected peptides are then fed into collision chamber 105 (now activated), where they collide with a gas, such as nitrogen, which breaks the peptides into fragments represented by triangles in FIG. 1. It is noted that at this point, again, the fragments could have an m/z ratio distribution across the entire range of the TOF analyzer 106. It is also noted that there a now many different fragments that relate to a number of different peptides that, in turn, relate to a number of different proteins.

After fragmentation, the fragments pass into time of flight analyzer 106. This module collects a number of fragments in grouping chamber 107 and starts a timer by “launching” the grouped fragments into accelerator 108. Detector 109 then detects the fragments and records the timer value between the “launch” and the detection. Since fragments are accelerated based on their m/z ratio, detector 109 essentially detects how many fragments are present for a specific m/z ratio. Simply put, heavy fragments with low charge are slower than light fragments with high charge and detector 109 detects the number of fragments at those ratios.

In summary, there are three filters that “sweep” or step across different ranges: First, the LC column 103 filters peptides depending how long they take to pass the column, independent of the m/z ratio and essentially sweeping across the retention time. The result at each point in time are peptides potentially distributed across the entire m/z range. Second, the quadrupole 104 filters peptides using their m/z ratio and steps through the entire range using m/z selection windows. It is assumed that the type of peptides eluted from LC column 103 is constant during one sweep of the selection windows. Since the selected peptides are fragmented, the fragments, again, are distributed across the entire m/z range. Third, the TOF analyzer 106 effectively sweeps across the m/z range of the fragments during one MS2 “shot” of the grouped fragments to record an intensity value for each m/z value. It is emphasized again that MS2 scans the fragments while MS1 scans the peptides.

It is noted here that there is a difference between peptide m/z ratios and fragment m/z ratios. During MS1, all peptides pass through to mass analyzer 106 where the “MS1 shot” (one per retention time index) is a measurement across the entire peptide m/z range. However, during MS2 the peptide m/z ratio is windowed in quadrupole 104, so that only peptides with a particular m/z range pass through and are fragmented. The fragment m/z ratio is then detected by TOF analyzer 106 where each “MS2 shot” (multiple windows per retention time index) is a measurement across the entire fragment m/z range. It is noted that a variety of different technologies exist to perform this type of spectroscopy including Orbitrap fragment detectors and other variants. Further details can also be found in: Christina Ludwig, Ludovic Gillet, George Rosenberger, Sabine Amon, Ben C Collins, Ruedi Aebersold, “Data-independent acquisition-based SWATH-MS for quantitative proteomics: a tutorial,” Molecular Systems Biology (2018) 14, e8126, which is incorporated herein by reference.

For each MS2 shot, the result is an intensity signal along an m/z axis. It is then possible to perform a peak detection algorithm to identify m/z values where the intensity shows a peak, in order to identify fragments that have been detected and reduce noise. Therefore, the output of the MS process may be a series of m/z values of fragments (where peaks were detected). The output may also include the intensity of the peak. The peak intensity, or the peak area, from individual proteins is here correlated to the amount of protein in the sample. However, the individual signal depends on the amino acid sequence of the peptide, on the complexity of the sample, and on the settings of the instrument. Therefore, standard mass spectrometry can only provide relative amounts of fragments/peptides, which does not enable quantitative comparisons to other samples.

Without wishing to be bound by theory, the present disclosure is based on the finding that using highly conserved peptides makes it possible to create sets or kits of peptide standards that can be used across a range of species. Embodiments of this disclosure demonstrate that these highly conserved peptides can be used as isotope labeled internal standards that can be used for absolute quantification. It is more convenient and less expensive to use peptides that are common across groups of species. On the basis of this finding, new methods of quantitative protein analysis and kits comprising conserved peptides for quantitative protein analysis are also disclosed herein.

Accordingly, in one aspect, the present disclosure provides a method for quantitative protein analysis of two or more species, the method comprising: determining a set of common peptides that are common for the two or more species, creating a set of isotope-labeled peptides out of the set of common peptides, adding a predefined amount of the labeled peptides to a sample from one of the two or more species, performing mass spectrometry to create first intensity values for sample peptides from the sample and second intensity values for the labeled peptides, and calculating a quantitative amount of the sample peptides based on the first intensity values and the second intensity values.

In at least one embodiment, adding the predefined amount of the labeled peptides may comprise adding the predefined amount of the labeled peptides to a sample from species in a group for which the set of common peptides was determined.

In at least one embodiment, determining the common peptides may be based on taxonomy comprising the two or more species. The taxonomy may represent evolutionary relationships.

In at least one embodiment, determining the set of common peptides may comprise: determining, by a computer system, digital data indicative of multiple species-specific sets of peptides based on digital sequence data from each of the respective species, and determining peptides that are common for the multiple sets of species-specific peptides.

In at least one embodiment, determining the set of common peptides is based on mass spectrometry data of the two or more species, the mass spectrometry data being indicative of multiple species-specific sets of peptides, and the method further comprises determining peptides that are common for the multiple sets of species-specific peptides.

In at least one embodiment, the species-specific sets of peptides comprise species-specific sets determined based on the digital sequence data and species-specific sets determined based on the mass spectrometry data.

Various embodiments disclosed herein may include a method of quantifying one or more protein complexes. The protein complex may be the same protein complex in two or more species. The protein complex may be a protein complex set out in, for example, Table 7 below.

In another aspect, the present disclosure provides a kit when used for quantitative protein analysis of two or more species, comprising two or more labeled peptides corresponding to peptides that are common between two or more species.

In at least one embodiment, the peptides common to the two or more species are selected from a set of common peptides.

In at least one embodiment, the common peptides are selected using a computational, a hybrid, or an empirical approach. In one example, the common peptides are selected using a computational approach. In another example, the common peptides are selected using a hybrid approach. In another example, the common peptides are selected using an empirical approach.

The kits comprising conserved sets of peptides may make up stand-alone kits for categories of organisms, such as the set of peptides for all vascular plants exemplified herein. The kits which are designed in a hierarchical taxonomic structure may be used alone or in combination. For example, one kit may contain peptides conserved across all eukaryotes. Another kit may contain peptides conserved across all vascular plants. Another kit may contain peptides conserved across all Rosids, a large group of dicot plants. Thus, for the study of species within the Rosids, all three kits could be combined to quantify large numbers of proteins. The hierarchical structure of kit designs minimizes the number of kits required to cover large swaths of genetic diversity.

Thus, in another aspect, the present disclosure provides a kit when used for quantitative protein analysis of two or more species of prokaryotes, comprising one or more labeled peptides selected from Table 1 herein.

In another aspect, the present disclosure provides a kit when used for quantitative protein analysis of two or more species of eukaryotes, comprising one or more labeled peptides selected from Table 2 herein.

In one example, the kit may be used for quantitative protein analysis of two or more species of vascular plants, comprising one or more labeled peptides selected from peptides in Tables 2 and 4 herein.

In another example, the kit may be used for quantitative protein analysis of two or more species of Rosids, comprising one or more labeled peptides selected from peptides in Tables 2, 3, and 4 herein.

In another aspect, the present disclosure provides a kit when used for quantitative protein analysis of two or more species of Rosids, comprising one or more labeled peptides selected from Table 3 herein.

In another aspect, the present disclosure provides a kit when used for quantitative protein analysis of two or more species of vascular plants, comprising one or more labeled peptides selected from Table 4 herein.

Embodiments of the disclosure may comprise usage of one or more kits described herein.

In another aspect, the present disclosure provides a kit comprising peptides that are labeled and selected from a set of peptides that are common for multiple species.

In another aspect, the present disclosure provides a computer-implemented method for quantitative protein analysis, the computer implemented method comprising: receiving mass spectrometry data comprising measurements with intensity values and corresponding mass-to-charge values, based on the mass-to-charge values, identifying: first measurements that relate to labeled peptides from a set of common peptides that are common for two or more plant species, and second measurements that relate to sample peptides from the set of common peptides, and calculating a quantitative amount of the sample peptides based on the intensity values of the first measurements and the intensity values of the second measurements.

In one example, the computer implemented further comprises determining the set of common peptides that are common for the two or more plant species.

Embodiments of the disclosure provide a method to identify peptides that are highly conserved across multiple species to be used as isotope labeled internal standards—it is the opposite of the normal approach of using unique peptides in quantitative proteomics. Using highly conserved peptides makes it possible to create sets or kits of peptide standards that can be used across a range of species, which saves users time and money. Unlike unique peptides, conserved peptides cannot differentiate between isoforms of the same protein. Instead, those isoforms are quantitatively measured as a group, which is sufficient in most experiments because the isoforms share a common molecular function. Users typically are interested in molecular functions related to biology and are only rarely interested in differentiating isoform amounts, which can be done separately and in addition to using sets of conserved peptides.

Thus, absolute quantitative proteomics produces far more useful results than relative quantification, but absolute quantification is expensive because peptides are normally designed on a species by species basis. The solution disclosed herein makes absolute quantification more convenient and less expensive by using peptides that are common across groups of species. For example, a user interested in studying grains could use a peptide kit that works across all species of grasses instead of designing and using different sets of peptides for each species of interest (e.g., wheat, rice, corn, etc.). In other words, the number of labeled peptides that are required for a range of species can contain a significantly smaller number of labeled peptides compared to using a separate kit for each species.

In one embodiment, sets of peptides make up stand-alone kits for categories of organisms, such as the set of peptides for all vascular plants exemplified below. In another embodiment, kits are designed in a hierarchical taxonomic structure to be used in combination. For example, one kit contains peptides conserved across all eukaryotes. A second kit contains peptides conserved across all vascular plants. A third kit contains peptides conserved across all Rosids, a large group of dicot plants. For the study of species within the Rosids, all three kits could be combined to quantify large numbers of proteins. The hierarchical structure of kit designs minimizes the number of kits required to cover large swaths of genetic diversity. In other words, instead of designing individual stand-alone kits for, e.g., each individual family or genus of organism (which would often contain redundant peptides with kits of close relative families and genera), the hierarchical design of kits covers large numbers of diverse species with a minimum number of non-redundant kits.

These and further and other objects and features of the invention are apparent in the disclosure, which includes the above and ongoing written specification, as well as the drawings.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art.

FIG. 1 illustrates mass spectrometry of protein samples, according to an embodiment of the disclosure.

FIG. 2 illustrates a computer system for performing quantitative protein analysis, according to an embodiment of the present disclosure.

FIG. 3 illustrates a method for quantitative protein analysis, according to an embodiment of the present disclosure.

FIG. 4 illustrates a taxonomy tree of bacteria, where the numbers indicate how many peptides are conserved among the tested species contained within the corresponding classification.

FIG. 5 illustrates a taxonomy tree of plants.

FIG. 6 illustrates the process of photosynthesis including the major complexes.

FIG. 7 illustrates molar ratios of 14 species' protein complexes, according to an embodiment of the present disclosure.

FIG. 8 illustrates ratios from the 14 species, but the ratios are relative to Rubisco and the proteins are related to the light-independent reactions of photosynthesis, according to an embodiment of the present disclosure.

FIGS. 9A-9B illustrate alignment of peptides of 10 different species against Arabidopsis as a reference sequence, according to an embodiment of the present disclosure.

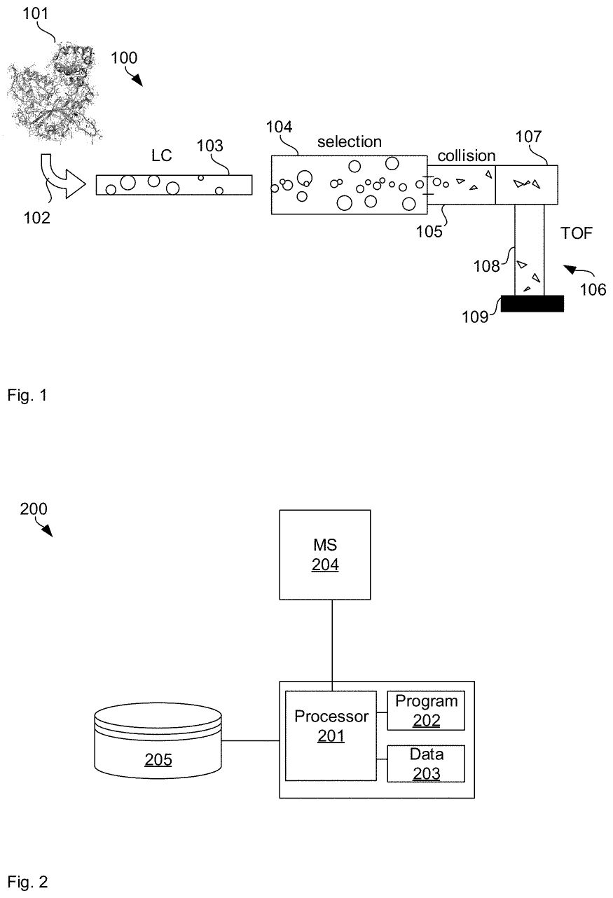

### DETAILED DESCRIPTION

The present invention is more fully described below with reference to the accompanying figures. The following description is exemplary in that several embodiments are described (e.g., by use of the terms “preferably,” “for example,” or “in one embodiment”); however, such should not be viewed as limiting or as setting forth the only embodiments of the present invention, as the invention encompasses other embodiments not specifically recited in this description, including alternatives, modifications, and equivalents within the spirit and scope of the invention. Further, the use of the terms “invention,” “present invention,” “embodiment,” and similar terms throughout the description are used broadly and not intended to mean that the invention requires, or is limited to, any particular aspect being described or that such description is the only manner in which the invention may be made or used. Additionally, the invention may be described in the context of specific applications; however, the invention may be used in a variety of applications not specifically described.

The embodiment(s) described, and references in the specification to “one embodiment”, “an embodiment”, “an example embodiment”, etc., indicate that the embodiment(s) described may include a particular feature, structure, or characteristic. Such phrases are not necessarily referring to the same embodiment. When a particular feature, structure, or characteristic is described in connection with an embodiment, persons skilled in the art may effect such feature, structure, or characteristic in connection with other embodiments whether or not explicitly described.

In the several figures, like reference numerals may be used for like elements having like functions even in different drawings. The embodiments described, and their detailed construction and elements, are merely provided to assist in a comprehensive understanding of the invention. Thus, it is apparent that the present invention can be carried out in a variety of ways, and does not require any of the specific features described herein. Also, well-known functions or constructions are not described in detail since they would obscure the invention with unnecessary detail. Any signal arrows in the drawings/figures should be considered only as exemplary, and not limiting, unless otherwise specifically noted. Further, the description is not to be taken in a limiting sense, but is made merely for the purpose of illustrating the general principles of the invention, since the scope of the invention is best defined by the appended claims.

It will be understood that, although the terms first, second, etc. may be used herein to describe various elements, these elements should not be limited by these terms. These terms are only used to distinguish one element from another. Purely as a non-limiting example, a first element could be termed a second element, and, similarly, a second element could be termed a first element, without departing from the scope of example embodiments. As used herein, the term “and/or” includes any and all combinations of one or more of the associated listed items. As used herein, the singular forms “a”, “an,” and “the” are intended to include the plural forms as well, unless the context clearly indicates otherwise. It should also be noted that, in some alternative implementations, the functions and/or acts noted may occur out of the order as represented in at least one of the several figures. Purely as a non-limiting example, two figures shown in succession may in fact be executed substantially concurrently or may sometimes be executed in the reverse order, depending upon the functionality and/or acts described or depicted.

As used herein, ranges are used herein in shorthand, so as to avoid having to list and describe each and every value within the range. Any appropriate value within the range can be selected, where appropriate, as the upper value, lower value, or the terminus of the range.

Unless indicated to the contrary, numerical parameters set forth herein are approximations that can vary depending upon the desired properties sought to be obtained. At the very least, and not as an attempt to limit the application of the doctrine of equivalents to the scope of any claims, each numerical parameter should be construed in light of the number of significant digits and ordinary rounding approaches.

The words “comprise”, “comprises”, and “comprising” are to be interpreted inclusively rather than exclusively. Likewise the terms “include”, “including” and “or” should all be construed to be inclusive, unless such a construction is clearly prohibited from the context. The terms “comprising” or “including” are intended to include embodiments encompassed by the terms “consisting essentially of” and “consisting of”. Similarly, the term “consisting essentially of” is intended to include embodiments encompassed by the term “consisting of”. Although having distinct meanings, the terms “comprising”, “having”, “containing” and “consisting of” may be replaced with one another throughout the description of the invention.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Terms such as, among others, “about,” “approximately,” “approaching,” or “substantially,” mean within an acceptable error for a particular value or numeric indication as determined by one of ordinary skill in the art, which depends in part on how the value is measured or determined. The aforementioned terms, when used with reference to a particular non-zero value or numeric indication, are intended to mean plus or minus 10% of that referenced numeric indication. As an example, the term “about 4” would include a range of 3.6 to 4.4. All numbers expressing dimensions, velocity, and so forth used in the specification are to be understood as being modified in all instances by the term “about.” Accordingly, unless indicated to the contrary, the numerical parameters set forth herein are approximations that can vary depending upon the desired properties sought to be obtained. At the very least, and not as an attempt to limit the application of the doctrine of equivalents to the scope of any claims, each numerical parameter should be construed in light of the number of significant digits and ordinary rounding approaches.

“Typically” or “optionally” means that the subsequently described event or circumstance may or may not occur, and that the description includes instances where said event or circumstance occurs and instances where it does not.

Wherever the phrase “for example,” “such as,” “including” and the like are used herein, the phrase “and without limitation” is understood to follow unless explicitly stated otherwise.

In general, the word “instructions,” as used herein, refers to logic embodied in hardware or firmware, or to a collection of software units, possibly having entry and exit points, written in a programming language, such as, but not limited to, Python, R, Rust, Go, SWIFT, Objective C, Java, JavaScript, Lua, C, C++, or C#. A software unit may be compiled and linked into an executable program, installed in a dynamic link library, or may be written in an interpreted programming language such as, but not limited to, Python, R, Ruby, JavaScript, or Perl. It will be appreciated that software units may be callable from other units or from themselves, and/or may be invoked in response to detected events or interrupts. Software units configured for execution on computing devices by their hardware processor(s) may be provided on a computer readable medium, such as a compact disc, digital video disc, flash drive, magnetic disc, or any other tangible medium, or as a digital download (and may be originally stored in a compressed or installable format that requires installation, decompression or decryption prior to execution). Such software code may be stored, partially or fully, on a memory device of the executing computing device, for execution by the computing device. Software instructions may be embedded in firmware, such as an EPROM. It will be further appreciated that hardware modules may be comprised of connected logic units, such as gates and flip-flops, and/or may be comprised of programmable units, such as programmable gate arrays or processors. Generally, the instructions described herein refer to logical modules that may be combined with other modules or divided into sub-modules despite their physical organization or storage. As used herein, the term “computer” is used in accordance with the full breadth of the term as understood by persons of ordinary skill in the art and includes, without limitation, desktop computers, laptop computers, tablets, servers, mainframe computers, smartphones, handheld computing devices, and the like.

In this disclosure, references are made to users performing certain steps or carrying out certain actions with their client computing devices/platforms. In general, such users and their computing devices are conceptually interchangeable. Therefore, it is to be understood that where an action is shown or described as being performed by a user, in various implementations and/or circumstances the action may be performed entirely by the user's computing device or by the user, using their computing device to a greater or lesser extent (e.g. a user may type out a response or input an action, or may choose from preselected responses or actions generated by the computing device). Similarly, where an action is shown or described as being carried out by a computing device, the action may be performed autonomously by that computing device or with more or less user input, in various circumstances and implementations.

In this disclosure, various implementations of a computer system architecture are possible, including, for instance, thin client (computing device for display and data entry) with fat server (cloud for app software, processing, and database), fat client (app software, processing, and display) with thin server (database), edge-fog-cloud computing, and other possible architectural implementations known in the art.

Generally, embodiments of the present disclosure provide a method for quantitative protein analysis. As set out above herein, the peak in the m/z intensity depends not only on the abundance of a protein, but also on the protein (peptide) structure and other factors. Therefore, it is inaccurate to infer quantities from relative peak values. For example, if a first fragment has peak at twice the intensity as a second fragment, it is not accurate to conclude that the corresponding first protein is twice as abundant than the second protein.

However, it is possible to label chemically synthesized peptides with isotopes or synthesize proteins that have labeled peptides. This way, the labeled synthesized peptide and the unlabeled natural peptide go through the same MS process and if they were equally abundant in the sample, they would show roughly equal intensity in their m/z peaks. It is noted that the peaks for the fragments of the labeled peptides are different from the unlabeled peptides due to the different mass of the isotopes. More information can be found in U.S. Pat. No. 7,501,286 entitled “ABSOLUTE QUANTIFICATION OF PROTEINS AND MODIFIED FORMS THEREOF BY MULTISTAGE MASS SPECTROMETRY,” which is incorporated herein by reference.

More particularly, the process of protein quantification comprises identifying a set of peptides that are to be analyzed quantitatively, combining the peptides to form a protein, synthesizing DNA to express that protein, providing the DNA to an organism (such as a bacterium) to express that protein while providing labeled pre-cursor molecules to the organism. Alternatively, the individual isotope labeled peptides are chemically synthesized. The labeled protein or peptides can then be added to the sample at a set amount (i.e., known abundance). The peaks of the natural peptides can then be “normalized” using the peaks of the labeled peptides. In other words, the quantitative abundance of the natural peptides can be calculated using the relative intensities between the peaks of the natural peptides and the peaks of the labeled peptides. Therefore, for example, if the amount of labeled peptide in the sample is 1 μmol/l and the peak of the natural peptide is ten times the peak of the labeled peptide, the abundance of the natural peptide is 10 μmol/l. More information on this process can be found in Julie M. Pratt, Deborah M. Simpson, Mary K. Doherty, Jenny Rivers, Simon J Gaskell, and Robert J Beynon: “Multiplexed absolute quantification for proteomics using concatenated signature peptides encoded by QconCAT genes,” Nature Protocols, Vol. 1 No. 2, 2006, which is incorporated herein by reference.

While the above process using QconCAT synthetic proteins comprised of concatenated peptides can provide quantitative abundances, it is difficult to use for quantitative proteomics across different species because protein sequences differ across species and manufacturing the labeled peptides is burdensome and inefficient as a high number of labeled peptides is required. Of course, this also increases costs to a level where quantitative protein analysis across multiple protein targets, multiple species, and experiments is practically unviable. More particularly, analyzing samples from different species may require a different set of labeled peptides and therefore re-starting the process from the beginning. This problem is less relevant, although still problematic, for humans and other mammals since they share a relatively high percentage sequence identity across conserved proteins. In other groups of organisms, however, the species are vastly different and therefore, a set of peptides that works for one species, is unlikely to yield useful results for a different species.

Embodiments of the disclosure provide a method for standardized quantitative analysis across different species. In particular, one or more embodiments provide a method to determine a set of peptides that can be used for quantitative protein analysis of all species of a selected group of species. This way, the set of labeled proteins only needs to be constructed once and can then be manufactured in a large amount, which reduces costs and complexity.

The species may be plant species. For example, a producer of grain seeds wants to achieve genetic gain through selection based on quantitative proteomic phenotyping. That producer may produce rice, barley and wheat. Instead of constructing one set of labeled peptides for each of these species, the producer can now use a single set of peptides that leads to useful quantitative data on all of those species.

In other examples, the species are prokaryotes, protocista, fungi, plants, and animals. When reference is made to “different species” herein, the species may be from the same kingdom or from different kingdoms. For example, the methods disclosed herein may be used for quantitative protein analysis of fungi and plants, or for quantitative protein analysis of only plants. Thus, in one example, the species may be prokaryotes. In another example, the species may be eukaryotes.

Peptide Selection

In order to construct labeled proteins that are usable for different species, methods disclosed herein may comprise a step of finding peptides that are common to the species of interest.

For example, a universal set of peptides may be constructed by finding peptides that are common across species from all existing plant divisions, such as Marchantiophyta (liverworts), Anthocerotophyta (hornworts), Bryophyta (mosses), Filicophyta (ferns), Sphenophyta (horsetails), Cycadophyta (cycads), Ginkgophyta (ginkgos), Pinophyta (conifers), Gnetophyta (gnetophytes), and the Magnoliophyta (Angiosperms, flowering plants). In other examples, the peptides are selected such that they are common across all groups of flowering plants (angiosperms).

In one example, the method comprises accessing a tree-structured taxonomy of plants, where each plant is represented by a node and connected to other nodes via common nodes (which may be ancestors in the tree), so that connected plant nodes form a Glade (a group of organisms believed to comprise all the evolutionary descendants of a common ancestor). The method then comprises receiving a selection of species of interest and then determining, based on the tree-structured taxonomy, the common node in the tree. This common node may be a common ancestor or an estimated common ancestor. From there, the method may sample representative species from the sub-trees below that ancestor. This may involve random sampling of species below the single common ancestor or identifying most relevant sub-trees in the taxonomy and choosing representative species of those sub-trees.

For each species, its comprehensive set of peptides is determined theoretically based on sequence data, empirically, or a combination of the two. There may be various different ways for determining a set of peptides for each species as set out in more detail below. For example, in cases where genome sequencing data is available for the species, it is possible to determine the peptides computationally from the genome by determining which proteins can be expressed from that genome and then determine which peptides are in those proteins according to cleavage characteristics of a selected protease such as trypsin. The genome may be retrieved from public databases or sequenced specifically for this purpose. In another example, the peptides are determined by mass spectrometry of the actual organisms. Therefore, once the species have been selected, biological samples of those species can be obtained and a set of peptides identified through mass spectrometry for each species.

In another example, an individual species may have a protein existing as different isoforms (due to alternative splicing, for example). In further examples, a group of species may have one or more common proteins that exist as homologs. As a result, the proteins have some different peptides and not all peptides are common across the group of species despite the common protein molecular function. For this reason, one or more embodiments of the disclosed method determines the set of peptides for a group of species.

Then, the method determines an intersection of the sets of peptides of the selected group of species. The intersection then contains the common peptides that can be used for labelling and quantitative protein analysis of the originally provided group of species.

For example, there are two different plant species I and II, which are different (fern and tomato). Both species have an example protein but different homologs of this protein. The homologs are functionally equivalent, but their sequences differ (except for the conserved parts). Species I has protein homolog A and species II has protein homolog B and it is desired to perform a quantitative protein analysis. In this example, homolog A has peptides abc and homolog B has peptides bef, so peptide b is in common, which means peptide b is evolutionarily conserved.

In other words, Species I has homolog A, which has peptides abc, while Species II has homolog B, which has peptides bef.

Then, the labeled peptides could be bhi. This would provide quantitative protein analysis because peptide b is in common and because of the 1:1:1 ratio of protein to peptide it is possible to quantify A as well as B (in the different samples). Also, if the protein exists in a protein complex of known and conserved stoichiometry, then the amounts of the complex and the additional proteins in the complex can be calculated.

Once the set of common peptides have been found, it is possible to perform the previously described method of creating QconCAT genes, expressing them into a labeled protein and sample that at known amounts together with samples from the species of interest. Alternatively, the set of common peptides could be chemically synthesized with isotope labeled amino acids.

Computational Approach

As mentioned above, there are different ways to determine the set of common peptides. First, there is a computational approach where the set of peptides is determined on digital data sources. More particularly, a digital representation of the genome of different plant species can be obtained and a computer system loads this representation, such as on random access memory (RAM) or hard disk drive (HDD).

The computer system starts with the first genome and scans the first genome to identify data patterns where trypsin would, if applied chemically, split a protein produced by the genome. More specifically, the computer system processes the digitally encoded DNA and replaces all occurrences of “T” (thymine) with “U” (uracil) to create a digitally encoded RNA. The computer system then translates the digitally encoded RNA into an amino acid sequence via the genetic code that converts each 3-mer of RNA (or “codon”), into one of 20 amino acids, which again are digitally encoded. The computing system then iterates over the amino acid sequence and every time the computer system encounters arginine or lysine, except when followed by proline, splits the amino acid sequence.

The resulting parts of the amino acid sequence resulting from the splits are the digitally encoded peptide sequences (i.e., sequences of amino acids). Given that there are 20 amino acids, each amino acid can be encoded by a 5-bit variable. Alternative encodings, such as one-hot 20 bit are also possible.

In at least one embodiment, available tools such as “translate” from the Swiss Bioinformatics Resource Portal (available at the expasy.org website) may also be used. While the above example relates to DNA as a starting point, other forms of digital sequence data, such as RNA, may be used as a starting point for the calculation of lists of proteins.

In at least one embodiment, the computer system stores the resulting list of peptides and repeats the process for the second genome and all further genomes of further species under consideration. This produces multiple lists of peptides including one list for each species. The computer system now processes the lists to find common elements. For example, the lists may be sorted, such as by converting the binary encoding of the amino acids into decimal numbers. Alternatively, the lists may be ordered by first amino acid, then by second amino acid, and so on similarly to how decimal numbers would be ordered sequentially by digits. The ordering speeds-up the search for common peptides because it is not necessary to iterate over the entire list.

In yet another example, the peptides may be stored in a database, such that each entry of a peptide in one of the lists has one entry in a database table. The computer system can then execute a query for common peptides, such as using a JOIN operation to find common peptides or an AND connection, like peptide_1 is in List_1 AND is in List_2. The advantage is that databases, such as SQL, have sophisticated mechanisms to optimize this search. In yet another example, Microsoft Excel can be used with the COUNTIF function to find common peptides.

The result of these processing methods is a list of peptides that are common for the two or more species under consideration. The advantage of this computational approach is that it requires no empirical steps, such as actual mass spectrometry data of biological samples. A potential disadvantage is that some identified peptides may be difficult to detect due to low expression levels in most species or other chemical behavior during mass spectrometry.

Empirical Approach

Aside from the computational method described above, it is possible to perform mass-spectrometry of samples from a reference species or group of species under consideration. This will yield a list of peptides per species and those lists can then be processed to identify common peptides as described above. It will be understood by those skilled in the art that any suitable mass-spectrometric instrument or mass-spectrometric data acquisition method may be used to identify common peptides. For example, SWATH analysis or other data independent methods may be used. In the case of data independent methods, peptide fragment data can be compared to a reference ion library created from a reference species.

In at least one embodiment, the reference ion library is created from data dependent acquisition analysis, and subsequent peptide-spectrum matching uses probabilistic scoring of a reference species for which comprehensive genome sequence data are available. Data independent acquisition is then used for additional species that may or may not have available genome sequence data. Comparisons of the data independent data from multiple species versus the reference ion library are scored probabilistically and identifications of conserved peptides are accepted or rejected based on a probability score such as false discovery rate. Similarly, data dependent acquisition mass spectrometry methods may be used.

In data dependent methods, the fragment ion spectra are either compared to a reference ion library as above or compared to peptide sequence data using peptide spectrum matching software that assigns peptide identifications to spectra. Those resulting peptide identifications can then be searched for conserved peptides across the multiple representative species of the taxonomic group of interest.

While this empirical approach only detects peptides that are observable, it requires the task of mass spectrometry of samples and therefore may be cumbersome and expensive, especially where a large number of species are considered for common peptides, such as ten species. The empirical approach does not require whole genome sequence data from more than one species. It only requires whole genome sequence data from the species that serves as the reference species. For example, Arabidopsis thaliana was the reference species in the empirical approach that identified the conserved peptides from vascular plants in Table 4. Data dependent A. thaliana peptide data were used with its full theoretical proteome, derived from its full genome sequence, to create an ion library. Then data independent data from peptides of additional 11 species of vascular plants were compared to the A. thaliana ion library.

Hybrid Approach

While the above sections describe a computational approach and an empirical approach, it is noted that not all representative species need to be processed by the same approach but a combination is possible. For example, one of the species may be analyzed empirically, which may even involve the use of a public database to obtain mass spectrometry data including a list of observed peptides from that one species. The other species can be analyzed using the computational approach. Since unobservable peptides are not included in the first list of peptides from the first species, they are automatically “filtered” from the computationally determined lists. This is so because all peptides in the final list of common peptides need to be in all of the lists, including the first that only contains observable peptides.

Computer Systems and Computer-Implemented Methods

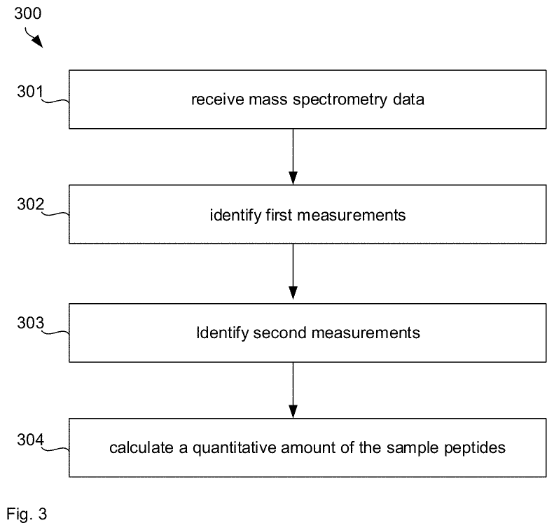

Turning now to FIG. 2, a computer system 200 for quantitative protein analysis is shown. Computer system 200 comprises a processor 201 connected to non-transitory (e.g. non-volatile) program memory 202 and data memory 203 (such as RAM or hard disk). Stored on program memory 202 is software code that, when executed by processor 201 causes processor 201 to execute the methods disclosed herein. In particular, processor 201 receives mass-spectrometry data from a mass spectrometer 204 and calculates quantities of proteins by performing, e.g., the steps of method 300 in FIG. 3. Processor 201 is also connected to database 205, which may store lists of peptides for two or more species or list of common peptides across two or more species.

FIG. 3 illustrates a computer-implemented method 300 for quantitative protein analysis of two or more species as performed by processor 201. First, processor 201 receives 301 mass spectrometry data. This data comprises measurements with intensity values and corresponding mass-to-charge values. The data may be provided in the form of a text file stored on data memory 203 or provided differently, such as through distributed data storage systems, e.g. Apache's Hadoop.

Based on the mass-to-charge values, processor 201 identifies 302 first measurements that relate to labeled peptides from a set of common peptides that are common for the two or more plant species. Processor 201 then identifies 303 second measurements that relate to sample peptides from the set of common peptides. These second measurements are for un-labeled peptides, which are naturally occurring in the sample and to be measured quantitatively. Finally, processor 201 calculates 304 a quantitative amount of the sample peptides based on the intensity values of the first measurements and the intensity values of the second measurements.

Calculating the quantitative amount in step 304 may be based on a known amount of labeled peptides that was added to the sample. This known amount may have been entered by the user through a user interface. In another example, the known amount is provided electronically by a dosing machine that automatically adds a pre-set amount of labeled peptides to the sample.

The quantitative amount may be relative to the added amount. For example, the processor 201 may calculate that the amount of unlabeled peptides is 10 times higher than the amount of unlabeled peptides. Processor 201 may output this result as a quantitative amount or may multiple the result with the known amount of added peptide to provide an absolute amount.

Importantly, processor 201 can repeat the receiving and identification steps for a different species but using the same set of common peptides, which is also referred herein as a “kit of labeled peptides.” As a result, the peptides of the second species can be quantitatively analyzed without the need to provide a different kit of labeled peptides. This makes the kit of peptides applicable for a wide range of species.

Even further, processor 201 can repeat the receiving and identification steps for a species that was not used for determining the common peptides. This can be done where a related species was used for determining the common peptides. In other words, there is a set of “training species” and processor 201 determines the set of common peptides for the training species as described above with reference to the computational, empirical and hybrid approaches. Processor 201 can then perform method 300 for one or more “test species” using the set of common peptides determined for the training species. Importantly, the test species does not have to be in the set of training species.

However, in examples described herein, the test species is within a space of species that is spanned by the training species in relation to a taxonomy of species, which may be an evolutionary relationship. In other words, the test species has a common ancestor in the taxonomy that is in the set of training species. In that sense, the kit of labeled peptides can be used for quantitative protein analysis of all species that have a common ancestor in the set of training species for which the kit was created.

The following examples further illustrate one or more embodiments of the present disclosure, but should not be construed as limiting the present disclosure, which is defined by the claims.

### EXAMPLES

Exemplary processes for the identification of conserved peptides and their uses in quantitative methods are set out in the Examples below.

### Example 1

### Computational Identification of Conserved Peptides in Bacteria

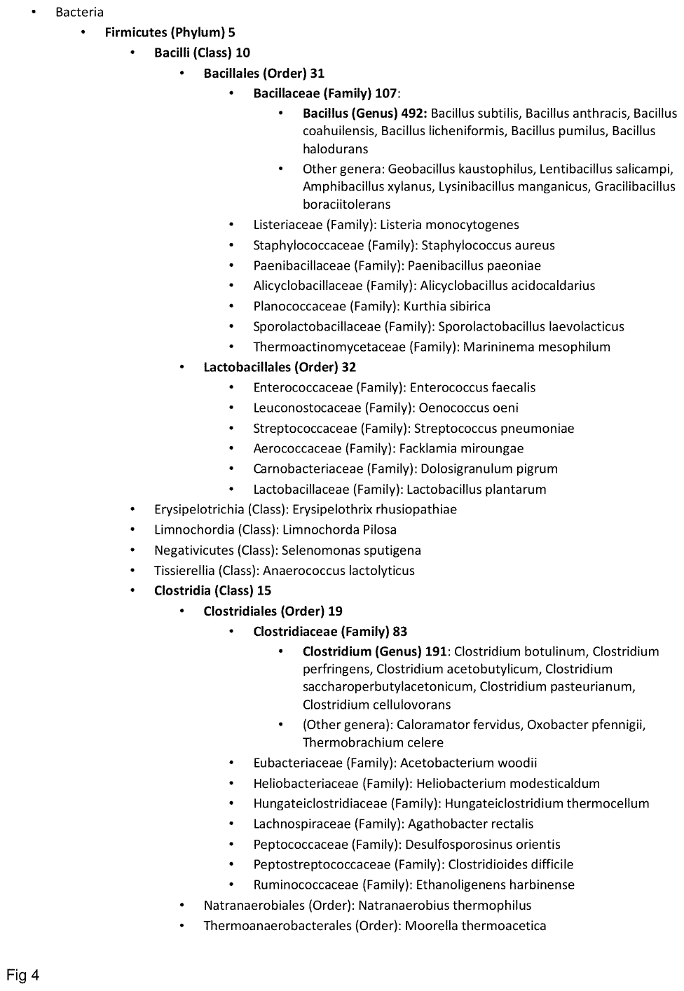

Conserved peptides were identified by theoretically digesting amino acid sequences from the bacterial genomes of 46 species of bacteria (FIG. 4). The species were selected to span the phylum Firmicutes, which is a large group of economically and medically significant bacteria.

Theoretical digestion of the FASTA amino acid sequences was carried out by using Protein Digestion Simulator with the following parameters: (a) no missed cleavages with trypsin cleavage defined as occurring at the C-terminal side of K or R residues and not at KP or RP; (b) a minimum of 7 residues; and (c) a minimum mass of 400 Da and a maximum of 6,000 Da.

The data was processed in Excel. Peptides in common among two or more species were identified using the COUNTIF function. For each pair or set of species in a comparison one was the reference—the set that was the range for the COUNTIF. Shared peptides returned COUNTIF values of 1 or more (more if the peptides occurred two or more times in the reference proteome).

The process was quickened by first, for a set of species, doing a simple pairwise comparison between two species to create a list of peptides in common between them, which was much shorter than the lists of total tryptic peptides for either species. Then, the resulting short list served as the reference list for additional comparisons.

The numbers in FIG. 4 indicate how many peptides are conserved among the tested species contained within the corresponding classification. Once a set of conserved peptides was found at a level of taxonomy, for example the 492 peptides conserved in the genus Bacillus, only those peptides were used for comparisons at the next higher level of taxonomy. In the Bacillus example, that means the 492 conserved peptides were used as the reference set for the family Bacillaceae—they were compared against the peptides of the representative species of the other genera in Bacillaceae. Then, the 107 conserved peptides of the Bacillaceae were used as the reference set for finding conserved peptides among the families that make up the Order Bacillales (see FIG. 4).

TABLE 1
Conserved peptides across bacterial species

| | Example protein in | Example protein in | SEQ ID |
|---|---|---|---|
| Sequence | Bacillus subtilis | Streptococcus pneumoniae | NO: |
| DVSGEGVQQALLK | sp\|P50866\|CLPX_BACSU | | 1 |
| NNPVLIGEPGVGK | sp\|O31673\|CLPE_BACSU | | 2 |
| RPIGSFIFLGPTGVGK | sp\|P37571\|CLPC_BACSU | | 3 |
| IIVDTYGGYAR | sp\|P54419\|METK_BACSU | | 4 |
| NFSIIAHIDHGK | sp\|P37949\|LEPA_BACSU | | 5 |
| VGIGPGSICTTR | sp\|P21879\|IMDH_BACSU | tr\|Q8DMX2\|Q8DMX2_STRR6 | 6 |
| AHILEGLR | sp\|P05653\|GYRA_BACSU | | 7 |
| EFTELGSGFK | sp\|P37474\|MFD_BACSU | | 8 |
| SVGELLQNQFR | sp\|P37870\|RPOB_BACSU | | 9 |
| LSALGPGGLTR | sp\|P37870\|RPOB_BACSU | sp\|Q8DNF0\|RPOB_STRR6 | 10 |
| LLHAIFGEK | sp\|P37870\|RPOB_BACSU | | 11 |
| STGPYSLVTQQPLGGK | sp\|P37870\|RPOB_BACSU | | 12 |
| AQFGGQR | sp\|P37870\|RPOB_BACSU | sp\|Q8DNF0\|RPOB_STRR6 | 13 |
| KPETINYR | sp\|P37871\|RPOC_BACSU | sp\|Q8DNF1\|RPOC_STRR6 | 14 |
| FATSDLNDLYR | sp\|P37871\|RPOC_BACSU | | 15 |
| GRPVTGPGNRPLK | sp\|P37871\|RPOC_BACSU | | 16 |
| SLSHMLK | sp\|P37871\|RPOC_BACSU | | 17 |
| IFGPVAR | sp\|P12875\|RL14_BACSU | sp\|P0A474\|RL14_STRR6 | 18 |
| GLMPNPK | sp\|Q06797\|RL1_BACSU | | 19 |
| ELIIGDR | sp\|P37808\|ATPA_BACSU | | 20 |
| DYLVPSR | sp\|O32038\|SYDND_BACSU | | 21 |
| KPNSALR | sp\|P21472\|RS12_BACSU | sp\|P0A4A8\|RS12_STRR6 | 22 |
| LVVSIAK | sp\|P06224\|SIGA_BACSU | sp\|P0A4J0\|SIGA_STRR6 | 23 |
| FSTYATWWIR | sp\|P06224\|SIGA_BACSU | sp\|P0A4J0\|SIGA_STRR6 | 24 |
| AIADQAR | sp\|P06224\|SIGA_BACSU | sp\|P0A4J0\|SIGA_STRR6 | 25 |
| IPVHMVETINK | sp\|P06224\|SIGA_BACSU | sp\|P0A4J0\|SIGA_STRR6 | 26 |
| FGLDDGR | sp\|P06224\|SIGA_BACSU | | 27 |
| ELPMEYAVEMNR | sp\|O32162\|SUFB_BACSU | | 28 |
| HYAHVDCPGHADYVK | sp\|P33166\|EFTU_BACSU | | 29 |
| GTVATGR | sp\|P33166\|EFTU_BACSU | | 30 |
| APGFGDR | sp\|P28598\|CH60_BACSU | sp\|P0A336\|CH60_STRR6 | 31 |
| IEDALNSTR | sp\|P28598\|CH60_BACSU | | 32 |
| GGGGYIR | | tr\|Q8DMZ9\|Q8DMZ9_STRR6 | 33 |
| TMDIGGDK | | tr\|Q8DPQ1\|Q8DPQ1_STRR6 | 34 |
| NTTIPTSK | | sp\|Q8CWT3\|DNAK_STRR6 | 35 |
| STLFNAITK | | tr\|Q8DRQ3\|Q8DRQ3_STRR6 | 36 |
| LLQGDVGSGK | | tr\|Q7ZAK6\|Q7ZAK6_STRR6 | 37 |
| GLLMGAR | | tr\|Q8DR06\|Q8DR06_STRR6 | 38 |
| DGLKPVQR | | tr\|Q8DQB4\|Q8DQB4_STRR6 | 39 |
| DGLKPVHR | | sp\|Q8DPM2\|GYRA_STRR6 | 40 |
| GGTDGSK | | sp\|Q8DQ05\|PEPT_STRR6 | 41 |
| VADNSGAR | | sp\|P0A474\|RL14_STRR6 | 42 |
| GYGTTLGNSLR | | sp\|P66709\|RPOA_STRR6 | 43 |
| LRPGEPK | | sp\|Q8DNF0\|RPOB_STRR6 | 44 |
| ALMGANMQR | | sp\|Q8DNF0\|RPOB_STRR6 | 45 |
| STPEGAR | | sp\|Q8CWN4\|SYD_STRR6 | 46 |
| EVIAFPK | | sp\|Q8CWN4\|SYD_STRR6 | 47 |
| GMTDTALK | | sp\|Q8DNF1\|RPOC_STRR6 | 48 |
| VLTDAAIR | | sp\|Q8DNF1\|RPOC_STRR6 | 49 |
| ENVIIGK | | sp\|Q8DNF1\|RPOC_STRR6 | 50 |
| VEFFGDEIDR | | sp\|Q8DPK7\|UVRB_STRR6 | 51 |
| GDWVISR | | sp\|Q8DNW4\|SYI_STRR6 | 52 |
| SSLAFDTLYAEGQR | | sp\|P63385\|UVRA_STRR6 | 53 |

### Example 2

### Computational Identification of Conserved Peptides in Eukaryotes

Amino acid sequences from the following Uniprot proteome entries were theoretically digested using Protein Digestion Simulator as above: Human (vertebrate animal), 75,069 sequences; Yeast—Saccharomyces cerevisiae (fungus), 6049 sequences; Nematode—Caenorhabditis elegans (invertebrate animal), 26,701 sequences; Arabidopsis thaliana (plant), 39,349 sequences; and Oomycete—Phytophthora infestans (member of a clade of oomycetes and protists distant from other eukaryotes), 17,514 sequences.

The digest outputs were processed in Excel. The yeast and phytophthora outputs were combined into one excel file. The organisms with the smallest proteomes were processed first

As above, Countif was used to determine if yeast peptides were present in phytophthora, resulting in 352 unique peptides conserved between yeast and phytophthora.

Countif was again used to identify peptides from Caenorhabditis elegans which are common to the 352 unique peptides identified between yeast and phytophthora. A total of 141 peptides conserved were identified in yeast, phytophthora and C. elegans.

Countif was again used to identify peptides from A. thaliana which are common to the 141 unique peptides identified between yeast, phytophthora and C. elegans. A total of 106 peptides conserved were identified in yeast, phytophthora, C. elegans and A. thaliana.

Countif was again used to identify human peptides which are common to the 106 unique peptides identified between yeast, phytophthora, C. elegans and A. thaliana . A total of 100 peptides conserved were identified in humans, yeast, phytophthora, C. elegans and A. thaliana . These are set out in Table 2, with example protein identifiers for yeast and Arabidopsis and example functional annotations from the MapMan annotation scheme for Arabidopsis.

TABLE 2
Conserved peptides in eukaryotes

| | | | MapMan annotation | |
|---|---|---|---|---|
| | | | [manual annotations | |
| | | | from TAIR proteins | |
| | | | names arc in | |
| | | TAIR10 | brackets when | SEQ |
| | | Arabidopsis | Mercator did not | ID |
| Sequence | Yeast Uniprot name | accession | provide annotation] | NO: |
| LTGMAFR | sp\|P00359\|G3P3_YEAST | AT1G79530 | Carbohydrate | 54 |
| | | | metabolism.plastidial | |
| | | | glycolysis.glyceralde | |
| | | | hyde 3-phosphate | |
| | | | dehydrogenase | |
| IGLFGGAGVGK | sp\|P00830\|ATPB_YEAST | AT5G08690 | Cellular | 55 |
| | | | respiration.oxidative | |
| | | | phosphorylation. ATP | |
| | | | synthase | |
| | | | complex.peripheral MF1 | |
| | | | subcomplex.subunit beta | |
| LQIWDTAGQER | sp\|P01123\|YPT1_YEAST | AT5G59840 | Vesicle | 56 |
| | | | trafficking.regulation | |
| | | | of membrane tethering | |
| | | | and fusion.RAB-GTPase | |
| | | | activities.E-class | |
| | | | RAB GTPase | |
| TITSSYYR | sp\|P01123\|YPT1_YEAST | AT4G17530 | Vesicle | 57 |
| | | | trafficking.regulation | |
| | | | of membrane tethering | |
| | | | and fusion.RAB-GTPase | |
| | | | activities.D-class RAB | |
| | | | GTPase | |
| EIQTAVR | sp\|P02294\|H2B2_YEAST | AT5G59910 | Chromatin | 58 |
| | | | organisation.histones. | |
| | | | histone (H2B) | |
| DNIQGITKPAIR | sp\|P02309\|H4_YEAST | AT5G59690 | Chromatin | 59 |
| | | | organisation.histones. | |
| | | | histone (H4) | |
| TLYGFGG | sp\|P02309\|H4_YEAST | AT5G59690 | Chromatin | 60 |
| | | | organisation.histonce. | |
| | | | histone (H4) | |
| ELISNASDALDK | sp\|P02829\|HSP82_YEAST | AT4G24190 | Protein | 61 |
| | | | homeostasis.protein | |
| | | | quality control.Hsp90 | |
| | | | chaperone system. | |
| | | | chaperone (Hsp90) | |
| STTTGHLIYK | sp\|P02994\|EF1A_YEAST | AT5G60390 | Protein biosynthesis. | 62 |
| | | | translation elongation. | |
| | | | eEF1 aminoacyl-tRNA | |
| | | | binding factor activity. | |
| | | | aminoacyl-tRNA binding | |
| | | | factor (cEF1A) | |
| LPLQDVYK | sp\|P02994\|EF1A_YEAST | AT5G60390 | Protein biosynthesis. | 63 |
| | | | translation elongation. | |
| | | | eEF1 aminoacyl-tRNA | |
| | | | binding factor | |
| | | | activity.aminoacyl- | |
| | | | tRNA binding factor | |
| | | | (eEF1A) | |
| IGGIGTVPVGR | sp\|P02994\|EF1A_YEAST | AT5G60390 | Protein biosynthesis. | 64 |
| | | | translation elongation. | |
| | | | cEF1 aminoacyl-tRNA | |
| | | | binding factor | |
| | | | activity.aminoacyl- | |
| | | | tRNA binding factor | |
| | | | (eEFlA) | |
| QTVAVGVIK | sp\|P02994\|EF1A_YEAST | AT5G60390 | Protein | 65 |
| | | | biosynthesis.translation | |
| | | | elongation.eEFl aminoacyl- | |
| | | | tRNA binding factor | |
| | | | activity.aminoacyl- | |
| | | | tRNA binding factor | |
| | | | (eEF1A) | |
| EGLIDTAVK | sp\|P04050\|RPB1_YEAST | AT4G35800 | RNA biosynthesis.DNA- | 66 |
| | | | dependent RNA polymerase | |
| | | | (Pol) complexes.Pol II | |
| | | | catalytic componcnts. | |
| | | | subunit 1 | |
| EGLVDTAVK | sp\|P04051\|RPC1_YEAST | AT5G60040 | RNA biosynthesis.DNA- | 67 |
| | | | dependent RNA polymerase | |
| | | | (Pol) complexes.Pol III | |
| | | | catalytic components. | |
| | | | subunit 1 | |
| EGIPPDQQR | sp\|P05759\|RS31_YEAST | AT5G37640 | Protein | 68 |
| | | | homeostasis.ubiquitin- | |
| | | | piuleasume system, | |
| | | | ubiquitin-fold protein | |
| | | | conjugation, ubiquitin | |
| | | | conjugation | |
| | | | (ubiquitylation). | |
| | | | ubiquitin-fold protein | |
| | | | (UBQ) | |
| ESTLHLVLR | sp\|P05759\|RS31_YEAST | AT5G37640 | Protein | 69 |
| | | | homeostasis.ubiquitin- | |
| | | | proteasome system. | |
| | | | ubiquitin-fold protein | |
| | | | conjugation.ubiquitin | |
| | | | conjugation | |
| | | | (ubiquitylation). | |
| | | | ubiquitin-fold protein | |
| | | | (UBQ) | |
| VADFGLAR | sp\|P06242\|KIN28_YEAST | AT5G07280 | Phytohormone | 70 |
| | | | action.signalling | |
| | | | peptides.NCRP (non- | |
| | | | cysteine-rich-peptide) | |
| | | | category.TDL-peptide | |
| | | | activity.TDL-peptide | |
| | | | receptor (EMS1/MSP1) | |
| MLDMGFEPQIR | sp\|P06634\|DED1_YEAST | AT5G63120 | RNA processing, pre- | 71 |
| | | | mRNA splicing.U2- | |
| | | | type-intron-specific | |
| | | | major spliceusuine.U1 | |
| | | | small nuclear | |
| | | | ribonucleoprotein | |
| | | | particle (snRNP).pre- | |
| | | | mRNA splicing regulator | |
| | | | (DDX5) | |
| SSALASK | sp\|P07259\|PYR1_YEAST | AT1G29900 | Amino acid metabolism. | 72 |
| | | | biosynthesis.glutamate | |
| | | | family.glutamate-derived | |
| | | | amino acids.arginine. | |
| | | | carbamoyl phosphate | |
| | | | synthetase heterodimer. | |
| | | | large subunit | |
| YDLTVPFAR | sp\|P07263\|SYH_YEAST | AT3G02760 | Protein | 73 |
| | | | biosynthesis.aminoacyl- | |
| | | | tRNA synthetase | |
| | | | activities.histidine- | |
| | | | tRNA ligase | |
| TITTAYYR | sp\|P07560\|SEC4_YEAST | AT5G59840 | Vesicle | 74 |
| | | | trafficking.regulation | |
| | | | of membrane tethering | |
| | | | and fusion.RAB-GTPase | |
| | | | activities.E-class | |
| | | | RAB GTPase | |
| QLWWGHR | sp\|P07806\|SYV_YEAST | AT5G16715 | Protein | 75 |
| | | | biosynthesis.aminoacyl- | |
| | | | tRNA synthetase | |
| | | | activities.valine- | |
| | | | tRNA ligasc | |
| AGVSQVLNR | sp\|P08518\|RPB2_YEAST | AT4G21710 | RNA biosynthesis.DNA- | 76 |
| | | | dependent RNA polymerase | |
| | | | (Pol) complexes.Pol II | |
| | | | catalytic components. | |
| | | | subunit 2 | |
| NTYQSAMGK | sp\|P08518\|RPB2_YEAST | AT4G21710 | RNA biosynthesis. DNA- | 77 |
| | | | dependent RNA polymerase | |
| | | | (Pol) complcxcs.Pol II | |
| | | | catalytic components. | |
| | | | subunit 2 | |
| LLLLGAGESGK | sp\|P08539\|GPA1_YEAST | AT2G26300 | Multi-process regulation. | 78 |
| | | | G-protein signalling. | |
| | | | heterotrimeric G-protein | |
| | | | complex.component alpha | |
| VEIIANDQGNR | sp\|P09435\|HSP73_YEAST | AT5G02500 | Protein homeostasis. | 79 |
| | | | protein quality control. | |
| | | | cytosolic Hsp70 chaperone | |
| | | | system.chaperone (Hsp70) | |
| TTPSYVAFTDTER | sp\|P09435\|HSP73_YEAST | AT1G16030 | Protein homeostasis. | 80 |
| | | | protein quality control. | |
| | | | cytosolic Hsp70 chaperone | |
| | | | system.chaperone (Hsp70) | |
| IINEPTAAAIAYGLDK | sp\|P09435\|HSP73_YEAST | AT5G42020 | [In 11 heat shock proteins | 81 |
| | | | in Arabidopsis] | |
| ITITNDK | sp\|P09435\|HSP73_YEAST | AT5G02490 | Protein homeostasis. | 82 |
| | | | protein quality control. | |
| | | | cytosolic Hsp70 chaperone | |
| | | | system.chaperone (Hsp70) | |
| FDLMYAK | sp\|P09733\|TBA1_YEAST | AT5G19770 | Cytoskeleton organisation. | 83 |
| | | | microtubular network.alpha- | |
| | | | beta-Tubulin heterodimer. | |
| | | | component alpha-Tubulin | |
| GGMQIFVK | sp\|P0CG63\|UBI4P_YEAST | AT5G37640 | Protein | 84 |
| | | | homeostasis.ubiquitin- | |
| | | | proteasome system. | |
| | | | ubiquitin-fold protein | |
| | | | conjugation, ubiquitin | |
| | | | conjugation | |
| | | | (ubiquitylation). | |
| | | | ubiquitin-fold protein | |
| | | | (UBQ) | |
| NTTIPTK | sp\|P0CS90\|HSP77_YEAST | AT5G02490 | Protein | 85 |
| | | | homeostasis.protein | |
| | | | quality control.cytosolic | |
| | | | Hsp70 chaperone system. | |
| | | | chaperone (Hsp70) | |
| VHGSLAR | sp\|P0CX34\|RS30B_YEAST | AT4G29390 | Protein biosynthesis. | 86 |
| | | | ribosome biogenesis. | |
| | | | small ribosomal subunit | |
| | | | (SSU).SSU | |
| | | | proteome.component | |
| | | | RPS30 | |
| ECADLWPR | sp\|P0CX42\|RL23B_YEAST | AT3G04400 | Protein biosynthesis. | 87 |
| | | | ribosome biogenesis.large | |
| | | | ribosomal subunit | |
| | | | (LSU).LSU | |
| | | | proteome.component RPL23 | |
| DELTLEGIK | sp\|P10081\|IF4A_YEAST | AT3G13920 | Protein biosynthesis. | 88 |
| | | | translation initiation. | |
| | | | mRNA loading.mRNA | |
| | | | unwinding factor (eIF4A) | |
| IDHYLGK | sp\|Pl1412\|G6PD_YEAST | AT5G40760 | Carbohydrate metabolism. | 89 |
| | | | oxidative pentose | |
| | | | phosphate pathway. | |
| | | | oxidative phase.glucosc-6- | |
| | | | phosphate dehydrogenase | |
| NAEYNPK | sp\|P13393\|TBP_YEAST | AT3G13445 | RNA biosynthesis.RNA | 90 |
| | | | polymerase II-dependent | |
| | | | transcription.transcription | |
| | | | initiation.TFIId basal | |
| | | | transcription regulation | |
| | | | complex.TATA-box-binding | |
| | | | component | |
| ALCTGEK | sp\|P14832\|CYPH_YEAST | AT5G13120 | Photosynthesis. | 91 |
| | | | photophosphorylation. | |
| | | | chlororespiration.NADH | |
| | | | dehydrogenase-like (NDH) | |
| | | | complex, lumen subcomplex | |
| | | | L.component PnsL5 | |
| DVIAFPK | sp\|P15179\|SYDM_YEAST | AT4G33760 | Protein biosynthesis. | 92 |
| | | | aminoacyl-tRNA | |
| | | | synthetase activities. | |
| | | | aspartate-tRNA ligase | |
| SAIGEGMTR | sp\|P16140\|VATB_YEAST | AT4G38510 | Solute transport.primary | 93 |
| | | | active transport.V-type | |
| | | | ATPase complex.peripheral | |
| | | | V1 subcomplex.subunit B | |
| DNNLLGK | sp\|P16474\|BIP_YEAST | AT5G02490 | Protein homeostasis. | 94 |
| | | | protein quality control. | |
| | | | cytosolic Hsp70 chaperone | |
| | | | system.chaperone (Hsp70) | |
| YFPTQALNFAFK | sp\|P18239\|ADT2_YEAST | AT5G13490 | Solute transport.carrier- | 95 |
| | | | mediated transport.solute | |
| | | | transporter (MTCC) | |
| APGFGDNR | sp\|P19882\|HSP60_YEAST | AT3G13860 | Protein homeostasis. | 96 |
| | | | proteinquality control. | |
| | | | Hsp60 chaperone system. | |
| | | | chaperone (Hsp60) | |
| AGAFDQLK | sp\|P20424\|SRP54_YEAST | AT5G49500 | Protein translocation. | 97 |
| | | | endoplasmic reticulum.co- | |
| | | | translational insertion | |
| | | | system.SRP (signal | |
| | | | recognition particle) | |
| | | | complex.component | |
| | | | SRP54 | |
| GYIDLSK | sp\|P20459\|IF2A_YEAST | AT5G05470 | Protein biosynthesis. | 98 |
| | | | translation initiation. | |
| | | | Pre-Initiation Complex | |
| | | | (PIC) module.eIF2 | |
| | | | Met-tRNA binding | |
| | | | factor activity.eIF2 | |
| | | | Met-tRNA binding factor | |
| | | | complex.component | |
| | | | eIF2-alpha | |
| TTLLHMLK | sp\|P20606\|SAR1_YEAST | AT3G62560 | Vesicle trafficking.Coat | 99 |
| | | | protein II (COPII) | |
| | | | coatomer machinery.coat | |
| | | | protein recruiting.GTPase | |
| | | | (Sar1) | |
| HITIFSPEGR | sp\|P21243\|PSA1_YEAST | AT2G05840 | Protein homeostasis. | 100 |
| | | | ubiquitin-proteasome | |
| | | | system.26S proteasome.20S | |
| | | | core particle.alpha-type | |
| | | | components.component | |
| | | | alpha type-1 | |
| NTYQCAMGK | sp\|P22276\|RPC2_YEAST | AT5G45140 | RNA biosynthesis.DNA- | 101 |
| | | | dependent RNA polymerase | |
| | | | (Pol) complexes.Pol III | |
| | | | catalytic components. | |
| | | | subunit 2 | |
| QITQVYGFYDECLR | sp\|P23595\|PP2A2_YEAST | AT5G55260 | Protein modification. | 102 |
| | | | phosphorylation. | |
| | | | serine/threonine protein | |
| | | | phosphatase superfamily. | |
| | | | PPP Fe—Zn-dependent | |
| | | | phosphatase families. | |
| | | | PP4-class phosphatase | |
| | | | complex.catalytic | |
| | | | component PP4c | |
| NIGISAHIDSGK | sp\|P25039\|EFGM_YEAST | AT2G45030 | Protein biosynthesis. | 103 |
| | | | organelle machinery. | |
| | | | translation elongation. | |
| | | | elongation factor (EF-G) | |
| GSLPWQGLK | sp\|P29295\|HRR25_YEAST | AT5G57015 | Protein modification. | 104 |
| | | | phosphorylation.CK | |
| | | | protein kinase | |
| | | | superfamily.protein | |
| | | | kinase (CKL) | |
| VAIHEAMEQQTISIAK | sp\|P29496\|MCM5_YEAST | AT2G07690 | Cell cycle organisation. | 105 |
| | | | DNA replication. | |
| | | | preinitiation.MCM | |
| | | | replicative DNA | |
| | | | helicase complex. | |
| | | | component MCM5 | |
| NMSVIAHVDHGK | sp\|P32324\|EF2_YEAST | AT1G56070 | Protein biosynthesis. | 106 |
| | | | translation elongation. | |
| | | | eEF2 mRNA-translocation | |
| | | | factor activity. mRNA- | |
| | | | translocation factor | |
| | | | (eEF2) | |
| QATINIGTIGHVAHGK | sp\|P32481\|IF2G_YEAST | AT4G18330 | Protein biosynthesis. | 107 |
| | | | translation initiation. | |
| | | | Pre-Initiation Complex | |
| | | | (PIC) module.eIF2 Met- | |
| | | | tRNA binding factor | |
| | | | activity.eIF2 Met-tRNA | |
| | | | binding factor complex. | |
| | | | component eIF2-gamma | |
| LGYANAK | sp\|P32481\|IF2G_YEAST | AT4G18330 | Protein biosynthesis. | 108 |
| | | | translation initiation. | |
| | | | Pre-Initiation Complex | |
| | | | (PIC) module.eIF2 Met- | |
| | | | tRNA binding factor | |
| | | | activity.eIF2 Met-tRNA | |
| | | | binding factor complex. | |
| | | | component eIF2-gamma | |
| QSLETICLLLAYK | sp\|P32598\|PP12_YEAST | AT5G59160 | Protein modification. | 109 |
| | | | phosphorylation. | |
| | | | serine/threonine | |
| | | | protein phosphatase | |
| | | | superfamily.PPP Fe—Zn- | |
| | | | dependent phosphatase | |
| | | | families.PP1-class | |
| | | | phosphatase | |
| GNHECASINR | sp\|P32598\|PP12_YEAST | AT5G59160 | Protein modification. | 110 |
| | | | phosphorylation. | |
| | | | serine/threonine | |
| | | | protein phosphatase | |
| | | | superfamily.PPP Fe—Zn- | |
| | | | dependent phosphatase | |
| | | | families.PP1-class | |
| | | | phosphatase | |
| IYGFYDECK | sp\|P32598\|PP12_YEAST | AT5G59160 | Protein modification. | 111 |
| | | | phosphorylation. | |
| | | | serine/threonine | |
| | | | protein phosphatase | |
| | | | superfamily.PPP Fe—Zn- | |
| | | | dependent phosphatase | |
| | | | families.PP1-class | |
| | | | phosphatase | |
| HLTGEFEK | sp\|P32836\|GSP2_YEAST | AT5G55190 | Protein translocation. | 112 |
| | | | nucleus. | |
| | | | nucleocytoplasmic | |
| | | | transport.Ran GTPase | |
| VCENIPIVLCGNK | sp\|P32836\|GSP2_YEAST | AT5G55190 | Protein translocation. | 113 |
| | | | nucleus. | |
| | | | nucleocytoplasmic | |
| | | | transport.Ran GTPase | |
| FQSLGVAFYR | sp\|P32939\|YPT7_YEAST | AT3G16100 | Vesicle trafficking. | 114 |
| | | | regulation of membrane | |
| | | | tethering and fusion. | |
| | | | RAB-GTPase activities. | |
| | | | G-class RAB GTPase | |
| YLGEGPR | sp\|P33298\|PRS6B_YEAST | AT5G58290 | Protein homeostasis, | 115 |
| | | | ubiquitin-proteasome | |
| | | | system. 26S proteasome. | |
| | | | 19S regulatory particle. | |
| | | | ATPase components. | |
| | | | regulatory component | |
| | | | RPT3 | |
| VIMATNR | sp\|P33298\|PRS6B_YEAST | AT5G58290 | Protein homeostasis. | 116 |
| | | | ubiquitin-proteasome | |
| | | | system.26S proteasome. | |
| | | | 19S regulatory particle. | |
| | | | ATPase components. | |
| | | | regulatory component | |
| | | | RPT3 | |
| VIGSELVQK | sp\|P33299\|PRS7_YEAST | AT1G53750 | Protein homeostasis. | 117 |
| | | | ubiquitin-proteasome | |
| | | | system.26S proteasome. | |
| | | | 19S regulatory particle. | |
| | | | ATPase components. | |
| | | | regulatory component | |
| | | | RPT1 | |
| YVGEGAR | sp\|P33299\|PRS7_YEAST | AT1G53750 | Protein homeostasis, | 118 |
| | | | ubiquitin-proteasome | |
| | | | system.26S proteasome. | |
| | | | 19S regulatory particle. | |
| | | | ATPase components. | |
| | | | regulatory component | |
| | | | RPT1 | |
| TGHSGTLDPK | sp\|P33322\|CBF5_YEAST | AT3G57150 | Protein biosynthesis. | 119 |
| | | | ribosome biogenesis. | |
| | | | rRNA biosynthesis.post- | |
| | | | transcriptional rRNA | |
| | | | modification. | |
| | | | pseudouridylation. | |
| | | | H/ACA small nucleolar | |
| | | | ribonucleoprotein | |
| | | | (snoRNP) rRNA | |
| | | | pseudouridylation | |
| | | | complex.pseudouridine | |
| | | | synthase component | |
| | | | Nap57/CBF5 | |
| FTLWWSPTINR | sp\|P33334\|PRP8_YEAST | AT4G38780 | RNA processing.pre- | 120 |
| | | | mRNA splicing.U2- | |
| | | | type-intron-specific | |
| | | | major spliceosome.U5 | |
| | | | small nuclear | |
| | | | ribonucleoprotein | |
| | | | particle (snRNP). | |
| | | | protein factor | |
| | | | (PRPF8/SUS2) | |
| ISLIQIFR | sp\|P33334\|PRP8_YEAST | AT4G38780 | RNA processing.pre- | 121 |
| | | | mRNA splicing.U2- | |
| | | | type-intron-spccific | |
| | | | major spliceosome.U5 | |
| | | | small nuclear | |
| | | | ribonucleoprotein | |
| | | | particle (snRNP). | |
| | | | protein factor | |
| | | | (PRPF8/SUS2) | |
| IIHTSVWAGQK | sp\|P33334\|PRP8_YEAST | AT4G38780 | RNA processing.pre- | 122 |
| | | | mRNA splicing.U2- | |
| | | | type-intron-specific | |
| | | | major spliceosome.U5 | |
| | | | small nuclear | |
| | | | ribonucleoprotein | |
| | | | particle (snRNP). | |
| | | | protein factor | |
| | | | (PRPF8/SUS2) | |
| LAEQAER | sp\|P34730\|BMH2YEAST | AT5G65430 | [In 16 regulatory | 123 |
| | | | proteins in | |
| | | | Arabidopsis] | |
| NLLSVAYK | sp\|P34730\|BMH2_YEAST | AT5G65430 | [In 16 regulatory | 124 |
| | | | proteins in | |
| | | | Arabidopsis] | |
| DSTLIMQLLR | sp\|P34730\|BMH2_YEAST | AT5G65430 | [In 25 regulatory | 125 |
| | | | proteins in | |
| | | | Arabidopsis] | |
| DIVFAASLYL | sp\|P35207\|SKI2_YEAST | AT1G59760 | RNA proccssing.RNA | 126 |
| | | | surveillance.exosome | |
| | | | complex.associated | |
| | | | co-factor activities. | |
| | | | Nuclear Exosome | |
| | | | Targeting (NEXT) | |
| | | | activation complex. | |
| | | | RNA helicase | |
| | | | component MTR4/HEN2 | |
| AQIWDTAGQER | sp\|P38555\|YPT31_YEAST | AT5G65270 | Vesicle trafficking. | 127 |
| | | | regulation of membrane | |
| | | | tethering and fusion. | |
| | | | RAB-GTPase activities. | |
| | | | A-class RAB GTPase | |
| AITSAYYR | sp\|P38555\|YPT31_YEAST | AT5G60860 | Vesicle trafficking. | 128 |
| | | | regulation of membrane | |
| | | | tethering and fusion. | |
| | | | RAB-GTPase activities. | |
| | | | A-class RAB GTPase | |
| LCDFGSAK | sp\|P38615\|RIM11_YEAST | AT5G26751 | Phytohormone action. | 129 |
| | | | brassinosteroid. | |
| | | | perception and signal | |
| | | | transduction.GSK3- | |
| | | | type protein kinase | |
| | | | (BIN2) | |
| IADFGLAK | sp\|P39009\|DUN1_YEAST | AT5G67080 | Protein modification. | 130 |
| | | | phosphorylation. | |
| | | | STE protein kinase | |
| | | | superfamily.protein | |
| | | | kinase (MAP3K- | |
| | | | MEKK) | |
| GANEATK | sp\|P39990\|SNU13_YEAST | AT5G20160 | RNA processing.pre- | 131 |
| | | | mRNA splicing.U2- | |
| | | | type-intron-specific | |
| | | | major spliceosome. | |
| | | | U4/U6 small nuclear | |
| | | | ribonucleoprotein | |
| | | | particle (snRNP). | |
| | | | protein factor | |
| | | | (NHP2L1/SNU13) | |
| LIGDAAK | sp\|P40150\|SSB2_YEAST | AT5G02500 | Protein homeostasis. | 132 |
| | | | protein quality | |
| | | | control.cytosolic | |
| | | | Hsp70 chaperone | |
| | | | system.chaperone | |
| | | | (Hsp70) | |
| DTQCGFK | sp\|P40350\|ALG5_YEAST | AT2G39630 | Protein modification. | 133 |
| | | | glycosylation.N-linked | |
| | | | glycosylalion.dolichol- | |
| | | | phosphate-glucose | |
| | | | synthase (ALG5) | |
| MLSCAGADR | sp\|P41805\|RL10_YEAST | AT1G66580 | Protein biosynthesis. | 134 |
| | | | ribosome biogenesis. | |
| | | | large ribosomal subunit | |
| | | | (LSU).LSU proteome. | |
| | | | component RPL10 | |
| ICDFGLAR | sp\|P41808\|SMK1_YEAST | AT5G19010 | Protein modification. | 135 |
| | | | phosphorylation. | |
| | | | CMGC protein kinase | |
| | | | superfamily.protein | |
| | | | kinase (MAPK) | |
| AVAVVVDPIQSVK | sp\|P43588\|RPN11_YEAST | AT5G23540 | Protein homeostasis. | 136 |
| | | | ubiquitin-proteasome | |
| | | | system.26S proteasome. | |
| | | | 19S regulatory | |
| | | | particle.non-ATPase | |
| | | | components.regulatory | |
| | | | component RPN11 | |
| VVIDAFR | sp\|P43588\|RPN11_YEAST | AT5G23540 | Protein homeostasis. | 137 |
| | | | ubiquitin-proteasome | |
| | | | system.26S proteasome. | |
| | | | 19S regulatory particle. | |
| | | | non-ATPase components. | |
| | | | regulatory component | |
| | | | RPN11 | |
| YMTDGMLLR | sp\|P53131\|PRP43_YEAST | AT4G16680 | [RNA helicase] | 138 |
| GVLLYGPPGTGK | sp\|P53549\|PRS10_YEAST | AT5G53540 | [RNA helicase] | 139 |
| YIGESAR | sp\|P53549\|PRS10_YEAST | AT1G45000 | Protein homeostasis. | 140 |
| | | | ubiquitin-proteasome | |
| | | | system.26S proteasome. | |
| | | | 19S regulatory particle. | |
| | | | ATPase components. | |
| | | | regulatory component | |
| | | | RPT4 | |
| LTSLGVIGALVK | sp\|P53829\|CAF40_YEAST | AT5G12980 | [Cell differentiation. | 141 |
| | | | Rcd1-like protein] | |
| GAFGEVR | sp\|P53894\|CBK1_YEAST | AT5G09890 | Protein modification. | 142 |
| | | | phosphorylation. | |
| | | | AGC protein kinase | |
| | | | superfamily.protein | |
| | | | kinase (AGC-VII/NDR) | |
| CATITPDEAR | sp\|P53982\|IDHH_YEAST | AT1G54340 | Enzyme classification. | 143 |
| | | | EC_l oxidoreductases. | |
| | | | EC_1.1 oxidoreductase | |
| | | | acting on CH—OH | |
| | | | group of donor | |
| SPNGTIR | sp\|P53982\|IDHH_YEAST | AT1G54340 | Enzyme classification. | 144 |
| | | | EC_1 oxidoreductases. | |
| | | | EC_1.1 oxidoreductase | |
| | | | acting on CH—OH | |
| | | | group of donor | |
| AGFAGDDAPR | sp\|P60010\|ACT_YEAST | AT5G59370 | Cytoskeleton organisation. | 145 |
| | | | microfilament network. | |
| | | | actin filament protein | |
| IWHHTFYNELR | sp\|P60010\|ACT_YEAST | AT5G59370 | Cytoskeleton organisation. | 146 |
| | | | microfilament network. | |
| | | | actin filament protein | |
| STELLIR | sp\|P61830\|H3_YEAST | AT5G10980 | Chromatin organisation. | 147 |
| | | | histones.histone (H3) | |
| EIAQDFK | sp\|P61830\|H3_YEAST | AT5G65350 | Chromatin organisation. | 148 |
| | | | histones. histone (H3) | |
| LGLTATLVR | sp\|Q00578\|RAD25_YEAST | AT5G41370 | DNA damage response. | 149 |
| | | | nucleotide excision | |
| | | | repair (NER).multi- | |
| | | | functional TFIIh | |
| | | | complex.core module. | |
| | | | subunit SSL2/XPB | |
| ELFVMAR | sp\|Q01939\|PRS8_YEAST | AT5G19990 | Protein homeostasis. | 150 |
| | | | ubiquitin-proteasome | |
| | | | system.26S proteasome. | |
| | | | 19S regulatory particle. | |
| | | | ATPase components. | |
| | | | regulatory component | |
| | | | RPT6 | |
| GTGLYELWK | sp\|Q02908\|ELP3_YEAST | AT5G50320 | RNA biosynthesis.RNA | 151 |
| | | | polymerase II-dependent | |
| | | | transcription. | |
| | | | transcription elongation. | |
| | | | ELONGATOR transcription | |
| | | | elongation complex. | |
| | | | component ELP3 | |
| TEALTQAFR | sp\|Q12464\|RUVB2_YEAST | AT3G49830 | Chromatin organisation. | 152 |
| | | | chromatin remodeling | |
| | | | complexes.SWR1/Nu | |
| | | | A4-shared helicase | |
| | | | (RVB) | |
| AGLQFPVGR | sp\|Q12692\|H2AZ_YEAST | AT5G54640 | Chromatin organisation. | 153 |
| | | | histones.histone (H2A) | |

### Example 3

### Hybrid Approach for the Identification of Conserved Peptides in Rosids

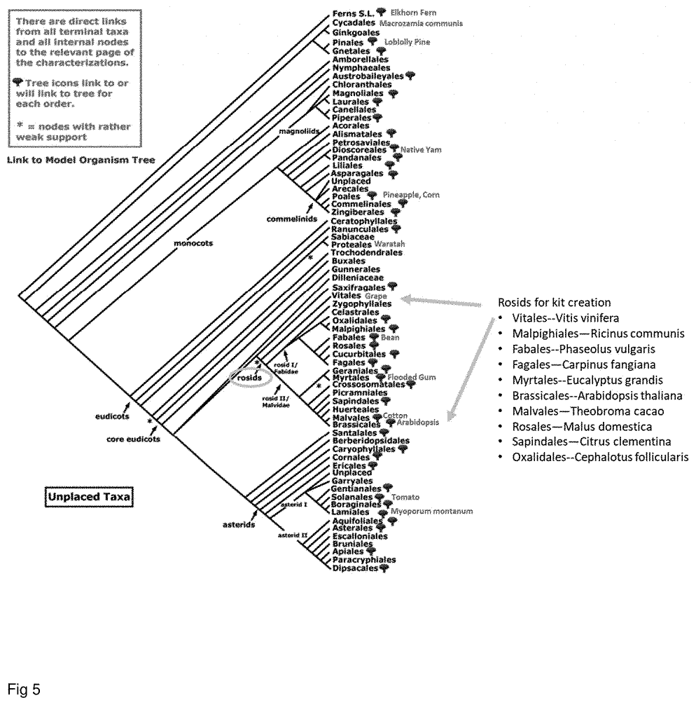

The Rosids is a large group of 17 orders of flowering plants (see FIG. 5). A list of 6647 conserved peptides among 10 species of Rosids (A. thaliana, Eucalyptus grandis, Ricinus communis, Phaseolus vulgaris, Vitis vinifera, Carpinus fangiana, Theobroma cacao, Malus domestica, Citrus clementina, and Cephalotus follicularis) were identified following the procedures outlined in Examples 1 and 2 above.

The list of 6647 conserved peptides were compared to the list of peptides identified in mass spectrometric experiments in the AraSpec database (Mergner et al., 2020). AraSpec has two large lists of reference peptides contained in ion libraries. One set contains phosphopeptides and the other contains non-phosphorylated peptides. For this analysis, the non-phosphorylated set was used and the redundant peptides, modified peptides and non-tryptic peptides were removed by comparing to a theoretical digest of A. thaliana.

Of these, 4647 peptides computationally found to be conserved among the ten species were also in AraSpec.

A list of peptides observed at FDR <0.01% was created from the four Rosid species in the dataset used to create the set of peptides for all vascular plants (Arabidopsis, Flooded gum, Grape, Bean) in Example 4 below. There were 647 peptides observed in all three replicates of the four species.

There were 231 peptides in common among all three sets: in the ten Rosids species theoretically, in AraSpec, and in the mass spec data from the four Rosids in triplicate.

Fifteen (15) of these peptides are found in all Eukaryotes (see Example 2). Thirty-six (36) of them are in the QconCATs for all vascular plants (see Example 4) and there are 5 peptides in the QconCATs that are found in all eukaryotes.

Not including the peptides in all eukaryotes and the QconCATs, there are 185 peptides that could be used for a Rosids kit.

In summary, the 185 Rosids peptides are: (1) theoretically conserved, (2) confirmed empirically from two sets of mass spectrometry data, (3) not in all eukaryotes, (4) not in the vascular plants prototype kit (QconCATs in Examples 4 through 7), (5) from 109 exemplary Arabidopsis proteins, (6) designed to be used with the eukaryotes kit and/or vascular plants kit, and (7) shown in Table 3 below.

TABLE 3
Conserved Rosid peptides

| | | | SEQ |
|---|---|---|---|
| | | Mercator or TAIR protein | ID |
| TAIR10 name | Sequence | description | NO: |
| AT1G03475.1 | NPFAPTLHFNYR | oxygen-dependent | 154 |
| | | coproporphyrinogen III | |
| | | oxidase (HemF) | |
| AT1G04420.1 | LNLFPGYMER | NAD(P)-linked | 155 |
| | | oxidoreductase superfamily | |
| | | protein | |
| AT1G06690.1 | FAALPWR | NAD(P)-linked | 156 |
| | | oxidoreductase superfamily | |
| | | protein | |
| AT1G15690.1 | AAVIGDTIGDPLK | proton-translocating | 157 |
| | | pyrophosphatase (VHP1) | |
| AT1G15690.2 | AADVGADLVGK | proton-translocating | 158 |
| | | pyrophosphatase (VHP1) | |
| AT1G15690.2 | TDALDAAGNTTAAIGK | proton-translocating | 159 |
| | | pyrophosphatase (VHP1) | |
| AT1G20010.1 | INVYYNEASGGR | component beta-Tubulin of | 160 |
| | | alpha-beta-Tubulin | |
| | | heterodimer | |
| AT1G29900.1 | VLILGGGPNR | large subunit of carbamoyl | 161 |
| | | phosphate synthetase | |
| | | heterodimer | |
| AT1G32060.1 | FYGEVTQQMLK | phosphoribulokinase | 162 |
| AT1G42970.1 | VVAWYDNEWGYSQR | glyceraldehyde 3-phosphate | 163 |
| | | dehydrogenase | |
| AT1G54340.1 | TIEAEAAHGTVTR | Peroxisomal isocitrate | 164 |
| | | dehydrogenase [NADP] | |
| | | OS = Arabidopsis thaliana | |
| | | (sp\|q9s1k0\|icdhx_arath: | |
| | | 872.0) & Enzyme | |
| | | classification.EC_1 | |
| | | oxidoreductases.EC_1.1 | |
| | | oxidoreductase acting on | |
| | | CH—OH group of | |
| | | donor(50.1.1:732.9) | |
| AT1G62750.1 | MDFPDPVIK | EF-G translation elongation | 165 |
| | | factor | |
| AT1G62750.1 | VEANVGAPQVNYR | EF-G translation elongation | 166 |
| | | factor | |
| AT1G62750.1 | LAQEDPSFHFSR | EF-G translation elongation | 167 |
| | | factor | |
| AT1G62750.1 | INIIDTPGHVDFTLEVER | EF-G translation elongation | 168 |
| | | factor | |
| AT1G62750.1 | IGEVHEGTATMDWMEQEQER | EF-G translation elongation | 169 |
| | | factor | |
| AT1G67280.2 | AFGMELLR | lactoyl-glutathione lyase | 170 |
| | | (GLX1) | |
| AT1G67280.2 | ITACLDPDGWK | lactoyl-glutathione lyase | 171 |
| | | (GLX1) | |
| AT1G67280.2 | GPTPEPLCQVMLR | lactoyl-glutathione lyase | 172 |
| | | (GLX1) | |
| AT1G70730.3 | LSGTGSEGATIR | cytosolic | 173 |
| | | phosphoglucomutase | |
| AT1G78900.2 | EDDLNEIVQLVGK | subunit A of V-type ATPase | 174 |
| | | peripheral V1 subcomplex | |
| AT1G78900.2 | HFPSVNWLISYSK | subunit A of V-type ATPase | 175 |
| | | peripheral V1 subcomplex | |
| AT1G78900.2 | VLDALFPSVLGGTCAIPGAFGCGK | subunit A of V-type ATPase | 176 |
| | | peripheral V1 subcomplex | |
| AT2G04030.2 | ELVSNASDALDK | chaperone (Hsp90) | 177 |
| AT2G28000.1 | VVNDGVTIAR | subunit alpha of Cpn60 | 178 |
| | | chaperonin complex | |
| AT2G30950.1 | FQMEPNTGVTFDDVAGVDEAK | component FtsH1\|2\|5\|6\|8 of | 179 |
| | | FtsH plastidial protease | |
| | | complexes | |
| AT2G39730.3 | VPLILGIWGGK | ATP-dependent activase | 180 |
| | | involved in RuBisCo | |
| | | regulation | |
| AT2G39730.3 | MCCLFINDLDAGAGR | ATP-dependent activase | 181 |
| | | involved in RuBisCo | |
| | | regulation | |
| AT2G39730.3 | MGINPIMMSAGELESGNAGEPAK | ATP-dependent activase | 182 |
| | | involved in RuBisCo | |
| | | regulation | |
| AT3G01340.2 | DVAWAPNLGLPK | scaffolding component | 183 |
| | | Sec13 of coat protein | |
| | | complex | |
| AT3G02360.1 | IGLAGLAVMGQNLALNIAEK | 6-phosphogluconate | 184 |
| | | dehydrogenase | |
| AT3G02450.1 | GVLLVGPPGTGK | component FtsHi of protein | 185 |
| | | translocation ATPase motor | |
| | | complex | |
| AT3G04400.2 | GSAITGPIGK | component RPL23 of LSU | 186 |
| | | proteome component | |
| AT3G04400.2 | NLYIISVK | component RPL23 of LSU | 187 |
| | | proteome component | |
| AT3G04400.2 | MSLGLPVAATVNCADNTGAK | component RPL23 of LSU | 188 |
| | | proteome component | |
| AT3G04770.2 | LLILTDPR | component RPSa of SSU | 189 |
| | | proteome | |
| AT3G05530.1 | ADILDPALMR | regulatory component RPT5 | 190 |
| | | of 26S proteasome | |
| AT3G09200.2 | VGSSEAALLAK | component RPP0 of LSU | 191 |
| | | proteome component | |
| AT3G11940.2 | QAVDISPLR | component RPS5 of SSU | 192 |
| | | proteome | |
| AT3G11940.2 | TIAECLADELINAAK | component RPS5 of SSU | 193 |
| | | proteome | |
| AT3G13120.2 | TMGPVPLPTK | component psRPS10 of | 194 |
| | | small ribosomal subunit | |
| | | proteome | |
| AT3G13930.1 | VIDGAIGAEWLK | component E2 of | 195 |
| | | mitochondrial pyruvate | |
| | | dehydrogenase complex | |
| AT3G15020.2 | LFGVTTLDVVR | mitochondrial NAD- | 196 |
| | | dependent malate | |
| | | dehydrogenase | |
| AT3G15020.2 | DDLFNINAGIVK | mitochondrial NAD- | 197 |
| | | dependent malate | |
| | | dehydrogenase | |
| AT3G16640.1 | VVDIVDTFR | translationally controlled | 198 |
| | | tumor protein | |
| AT3G26650.1 | LLDASHR | glyceraldehyde 3-phosphate | 199 |
| | | dehydrogenase | |
| AT3G26650.1 | VAINGFGR | glyceraldehyde 3-phosphate | 200 |
| | | dehydrogenase | |
| AT3G26650.1 | GTMTTTHSYTGDQR | glyceraldehyde 3-phosphate | 201 |
| | | dehydrogenase | |
| AT3G26650.1 | VIAWYDNEWGYSQR | glyceraldehyde 3-phosphate | 202 |
| | | dehydrogenase | |
| AT3G46970.1 | MSILSTAGSGK | cytosolic alpha-glucan | 203 |
| | | phosphorylase | |
| AT3G54050.2 | QIASLVQR | fructose- 1,6-bispho sphatase | 204 |
| AT3G54050.2 | TLLYGGIYGYPR | fructose- 1,6-bispho sphatase | 205 |
| AT3G58610.3 | GHSYSEIINESVIESVDSLNPFMHAR | ketol-acid reductoisomerase | 206 |
| AT3G63140.1 | DCEEWFFDR | endoribonuclease (CSP41) | 207 |
| AT3G63410.1 | NVTILDQSPHQLAK | MSBQ-methyltransferase | 208 |
| | | (APG1) | |
| AT4G01800.2 | VENYFFDIR | component SecA1 of | 209 |
| | | thylakoid membrane Sec1 | |
| | | translocation system | |
| AT4G02080.1 | ILFLGLDNAGK | GTPase (Sar1) | 210 |
| AT4G02770.1 | EQCLALGTR | component PsaD of PS-I | 211 |
| | | complex | |
| AT4G02770.1 | EQIFEMPTGGAAIMR | component PsaD of PS-I | 212 |
| | | complex | |
| AT4G04640.1 | VELLYTK | subunit gamma of | 213 |
| | | peripheral CF1 subcomplex | |
| | | of ATP synthase complex | |
| AT4G09000.2 | QAFDEAIAELDTLGEESYK | general regulatory factor 1 | 214 |
| AT4G13570.2 | GDEELDTLIK | histone (H2A) | 215 |
| AT4G13940.4 | HSLPDGLMR | S-adenosyl homocysteine | 216 |
| | | hydrolase | |
| AT4G15000.2 | YTLDVDLK | component RPL27 of LSU | 217 |
| | | proteome component | |
| AT4G17170.1 | YIIIGDTGVGK | B-class RAB GTPase | 218 |
| AT4G20360.1 | MVMPGDR | EF-Tu translation | 219 |
| | | elongation factor | |
| AT4G20360.1 | YDEIDAAPEER | EF-Tu translation | 220 |
| | | elongation factor | |
| AT4G20360.1 | GITINTATVEYETENR | EF-Tu translation | 221 |
| | | elongation factor | |
| AT4G20360.1 | HSPFFAGYRPQFYMR | EF-Tu translation | 222 |
| | | elongation factor | |
| AT4G24190.2 | FGWSANMER | chaperone (Hsp90) | 223 |
| AT4G26970.1 | ILLESAIR | aconitase | 224 |
| AT4G27700.1 | EWTAWDIAR | Rhodanese/Cell cycle | 225 |
| | | control phosphatase | |
| | | superfamily protein | |
| AT4G29060.2 | EETGAGMMDCK | EF-Ts translation elongation | 226 |
| | | factor | |
| AT4G30190.2 | ELSEIAEQAK | P3A-type proton- | 227 |
| | | translocating ATPase | |
| | | (AHA) | |
| AT4G30920.1 | TIEVNNTDAEGR | M17-class leucyl | 228 |
| | | aminopeptidase (LAP) | |
| AT4G33010.1 | VDNVYGDR | glycine dehydrogenase | 229 |
| | | component P-protein of | |
| | | glycine cleavage system | |
| AT4G33010.2 | TFCIPHGGGGPGMGPIGVK | glycine dehydrogenase | 230 |
| | | component P-protein of | |
| | | glycine cleavage system | |
| AT4G34450.1 | SIATLAITTLLK | subunit gamma of cargo | 231 |
| | | adaptor F-subcomplex | |
| AT4G35650.1 | LADGLFLESCR | regulatory component of | 232 |
| | | isocitrate dehydrogenase | |
| | | heterodimer | |
| AT4G35830.1 | VLLQDFTGVPAVVDLACMR | aconitase | 233 |
| AT4G35830.2 | TSLAPGSGVVTK | aconitase | 234 |
| AT4G38510.5 | IALTTAEYLAYECGK | subunit B of V-type ATPase | 235 |
| | | peripheral V1 subcomplex | |
| AT4G38510.5 | IPLFSAAGLPHNEIAAQICR | subunit B of V-type ATPase | 236 |
| | | peripheral V1 subcomplex | |
| AT4G38970.1 | ALQNTCLK | fructose 1,6-bisphosphate | 237 |
| | | aldolase | |
| AT5G03340.1 | DFSTAILER | platform ATPase (CDC48) | 238 |
| AT5G03340.1 | GILLYGPPGSGK | platform ATPase (CDC48) | 239 |
| AT5G03340.1 | IVS QLLTLMDGLK | platform ATPase (CDC48) | 240 |
| AT5G04140.2 | WPLAQPMR | Fd-dependent glutamate | 241 |
| | | synthase | |
| AT5G04140.2 | FCTGGMSLGAISR | Fd-dependent glutamate | 242 |
| | | synthase | |
| AT5G08690.1 | EMIESGVIK | subunit beta of ATP | 243 |
| | | synthase peripheral MF1 | |
| | | subcomplex | |
| AT5G08690.1 | TVLIMELINNVAK | subunit beta of ATP | 244 |
| | | synthase peripheral MF1 | |
| | | subcomplex | |
| AT5G08690.1 | FTQANSEVSALLGR | subunit beta of ATP | 245 |
| | | synthase peripheral MF1 | |
| | | subcomplex | |
| AT5G08690.1 | CALVYGQMNEPPGAR | subunit beta of ATP | 246 |
| | | synthase peripheral MF1 | |
| | | subcomplex | |
| AT5G09660.4 | ANTFVAEVLGLDPR | peroxisomal NAD- | 247 |
| | | dependent malate | |
| | | dehydrogenase | |
| AT5G09810.1 | YPIEHGIVSNWDDMEK | actin filament protein | 248 |
| AT5G10860.1 | VGDIMTEENK | Cystathionine beta- synthase | 249 |
| | | (CBS) family protein | |
| AT5G11520.1 | LNLGVGAYR | aspartate aminotransferase | 250 |
| AT5G13490.2 | TAAAPIER | solute transporter (MTCC) | 251 |
| AT5G13490.2 | MMMTSGEAVK | solute transporter (MTCC) | 252 |
| AT5G14300.1 | DLQMVNLTLR | prohibitin 5 | 253 |
| AT5G14670.1 | ILMVGLDAAGK | ARF-GTPase | 254 |
| AT5G14670.1 | NISFTVWDVGGQDK | ARF-GTPase | 255 |
| AT5G15200.2 | IFEGEALLR | component RPS9 of SSU | 256 |
| | | proteome | |
| AT5G15650.1 | DELDIVIPTIR | UDP-L-arabinose mutase | 257 |
| AT5G16440.1 | AFSVFLFNSK | isopentenyl diphosphate | 258 |
| | | isomerase | |
| AT5G16990.1 | NLYLSCDPYMR | NADP-dependent alkenal | 259 |
| | | double bond reductase P2 | |
| | | OS = Arabidopsis thaliana | |
| | | (sp\|q39173\|p2_arath: | |
| | | 704.0) & Enzyme | |
| | | classification.EC_1 | |
| | | oxidoreductases.EC_1.3 | |
| | | oxidoreductase acting on | |
| | | CH—CH group of | |
| | | donor(50.1.3:295.5) | |
| AT5G17920.2 | YLFAGVVDGR | methyl-tetrahydrofolate- | 260 |
| | | dependent methionine | |
| | | synthase | |
| AT5G18380.2 | TLLVADPR | component RPS16 of SSU | 261 |
| | | proteome | |
| AT5G19780.1 | AVFVDLEPTVIDEVR | component alpha-Tubulin of | 262 |
| | | alpha-beta-Tubulin | |
| | | heterodimer | |
| AT5G20980.2 | SWLAFAAQK | methyl-tetrahydrofolate- | 263 |
| | | dependent methionine | |
| | | synthase | |
| AT5G20980.2 | YGAGIGPGVYDIHSPR | methyl-tetrahydrofolate- | 264 |
| | | dependent methionine | |
| | | synthase | |
| AT5G20980.2 | GMLTGPVTILNWSFVR | methyl-tetrahydrofolate- | 265 |
| | | dependent methionine | |
| | | synthase | |
| AT5G23120.1 | GFGILDVGYR | HCF136 protein involved in | 266 |
| | | PS-II assembly | |
| AT5G23860.2 | LAVNLIPFPR | component beta-Tubulin of | 267 |
| | | alpha-beta-Tubulin | |
| | | heterodimer | |
| AT5G23860.2 | LHFFMVGFAPLTSR | component beta-Tubulin of | 268 |
| | | alpha-beta-Tubulin | |
| | | heterodimer | |
| AT5G23860.2 | GHYTEGAELIDSVLDVVR | component beta-Tubulin of | 269 |
| | | alpha-beta-Tubulin | |
| | | heterodimer | |
| AT5G25880.1 | IWLVDSK | cytosolic NADP-dependent | 270 |
| | | malic enzyme | |
| AT5G25880.1 | ILGLGDLGCQGMGIPVGK | cytosolic NADP-dependent | 271 |
| | | malic enzyme | |
| AT5G26780.2 | GAMIFFR | serine | 272 |
| | | hydroxymethyltransferase | |
| AT5G26780.2 | MGTPALTSR | serine | 273 |
| | | hydroxymethyltransferase | |
| AT5G26780.2 | LIVAGASAYAR | serine | 274 |
| | | hydroxymethyltransferase | |
| AT5G26780.2 | NTVPGDVSAMVPGGIR | serine | 275 |
| | | hydroxymethyltransferase | |
| AT5G26780.2 | ISAVSIFFETMPYR | serine | 276 |
| | | hydroxymethyltransferase | |
| AT5G30510.1 | AEEMAQTFR | component psRPS1 of small | 277 |
| | | ribosomal subunit proteome | |
| AT5G35530.1 | GLCAIAQAESLR | component RPS3 of SSU | 278 |
| | | proteome | |
| AT5G36700.4 | ENPGCLFIATNR | phosphoglycolate | 279 |
| | | phosphatase | |
| AT5G37600.1 | WNYDGSSTGQAPGEDSEVILYPQAIFK | cytosolic glutamine | 280 |
| | | synthetase (GLN1 ) | |
| AT5G38480.2 | YEEMVEFMEK | general regulatory factor 3 | 281 |
| AT5G41670.2 | GFPISVYNR | 6-phosphogluconate | 282 |
| | | dehydrogenase | |
| AT5G42270.1 | LESGLYSR | component FtsH1\|2\|5\|6\|8 of | 283 |
| | | FtsH plastidial protease | |
| | | complexes | |
| AT5G42270.1 | DEISDALER | component FtsH1\|2\|5\|6\|8 of | 284 |
| | | FtsH plastidial protease | |
| | | complexes | |
| AT5G42270.1 | LELQEVVDFLK | component FtsH1\|2\|5\|6\|8 of | 285 |
| | | FtsH plastidial protease | |
| | | complexes | |
| AT5G42270.1 | TPGFTGADLQNLMNEAAILAAR | component FtsH1\|2\|5\|6\|8 of | 286 |
| | | FtsH plastidial protease | |
| | | complexes | |
| AT5G45775.2 | YEGVILNK | component RPL11 of LSU | 287 |
| | | proteome component | |
| AT5G45775.2 | AMQLLESGLK | component RPL11 of LSU | 288 |
| | | proteome component | |
| AT5G45930.1 | IGGVMIMGDR | component CHL-I of | 289 |
| | | magnesium-chelatase | |
| | | complex | |
| AT5G45930.1 | INMVDLPLGATEDR | component CHL-I of | 290 |
| | | magnesium-chelatase | |
| | | complex | |
| AT5G45930.1 | FILIGSGNPEEGELRPQLLDR | component CHL-I of | 291 |
| | | magnesium-chelatase | |
| | | complex | |
| AT5G48300.1 | MLDADVTDSVIGEGCVIK | ADP-glucose | 292 |
| | | pyrophosphorylase | |
| AT5G49910.1 | IAGLEVLR | chaperone (cpHsc70) | 293 |
| AT5G49910.1 | FEELCSDLLDR | chaperone (cpHsc70) | 294 |
| AT5G49910.1 | QFAAEEISAQVLR | chaperone (cpHsc70) | 295 |
| AT5G50920.1 | LDEMIVFR | chaperone component ClpC | 296 |
| | | of chloroplast Clp-type | |
| | | protease complex | |
| AT5G50920.1 | LDMSEFMER | chaperone component ClpC | 297 |
| | | of chloroplast Clp-type | |
| | | protease complex | |
| AT5G50920.1 | VIMLAQEEAR | chaperone component ClpC | 298 |
| | | of chloroplast Clp-type | |
| | | protease complex | |
| AT5G50920.1 | IGFDLDYDEK | chaperone component ClpC | 299 |
| | | of chloroplast Clp-type | |
| | | protease complex | |
| AT5G50920.1 | VITLDMGLLVAGTK | chaperone component ClpC | 300 |
| | | of chloroplast Clp-type | |
| | | protease complex | |
| AT5G50920.1 | ALAAYYFGSEEAMIR | chaperone component ClpC | 301 |
| | | of chloroplast Clp-type | |
| | | protease complex | |
| AT5G50920.1 | NTLLIMTSNVGSSVIEK | chaperone component ClpC | 302 |
| | | of chloroplast Clp-type | |
| | | protease complex | |
| AT5G50920.1 | AHPDVFNMMLQILEDGR | chaperone component ClpC | 303 |
| | | of chloroplast Clp-type | |
| | | protease complex | |
| AT5G50920.1 | LIGSPPGYVGYTEGGQLTEAVR | chaperone component ClpC | 304 |
| | | of chloroplast Clp-type | |
| | | protease complex | |
| AT5G55070.1 | GLVVPVIR | component E2 of 2- | 305 |
| | | oxoglutarate dehydrogenase | |
| | | complex | |
| AT5G56030.2 | EEYAAFYK | chaperone (Hsp90) | 306 |
| AT5G56030.2 | AVENSPFLEK | chaperone (Hsp90) | 307 |
| AT5G56030.2 | ADLVNNLGTIAR | chaperone (Hsp90) | 308 |
| AT5G56030.2 | EDQLEYLEER | chaperone (Hsp90) | 309 |
| AT5G56030.2 | GIVDSEDLPLNISR | chaperone (Hsp90) | 310 |
| AT5G56500.2 | VEDALNATK | subunit beta of Cpn60 | 311 |
| | | chaperonin complex | |
| AT5G56500.2 | VVAAGANPVLITR | subunit beta of Cpn60 | 312 |
| | | chaperonin complex | |
| AT5G56500.2 | EVELEDPVENIGAK | subunit beta of Cpn60 | 313 |
| | | chaperonin complex | |
| AT5G56500.2 | AAVEEGIVVGGGCTLLR | subunit beta of Cpn60 | 314 |
| | | chaperonin complex | |
| AT5G56500.2 | LSGGVAVIQVGAQTETELK | subunit beta of Cpn60 | 315 |
| | | chaperonin complex | |
| AT5G57350.2 | LGDIIPADAR | P3A-type proton- | 316 |
| | | translocating ATPase | |
| | | (AHA) | |
| AT5G57350.2 | ADGFAGVFPEHK | P3A-type proton- | 317 |
| | | translocating ATPase | |
| | | (AHA) | |
| AT5G57350.2 | ADIGIAVADATDAAR | P3A-type proton- | 318 |
| | | translocating ATPase | |
| | | (AHA) | |
| AT5G57350.2 | MTAIEEMAGMDVLCSDK | P3A-type proton- | 319 |
| | | translocating ATPase | |
| | | (AHA) | |
| AT5G59370.2 | GYSFTTTAER | actin filament protein | 320 |
| AT5G59370.2 | HTGVMVGMGQK | actin filament protein | 321 |
| AT5G59370.2 | VAPEEHPVLLTEAPLNPK | actin filament protein | 322 |
| AT5G59840.1 | LLLIGDSGVGK | E-class RAB GTPase | 323 |
| AT5G59850.1 | IVVELNGR | component RPS15a of SSU | 324 |
| | | proteome | |
| AT5G59910.1 | LVLPGELAK | histone (H2B) | 325 |
| AT5G59910.1 | AMGIMNSFINDIFEK | histone (H2B) | 326 |
| AT5G59970.1 | DAVTYTEHAR | histone (H4) | 327 |
| AT5G59970.1 | ISGLIYEETR | histone (H4) | 328 |
| AT5G59970.1 | TVTAMDVVYALK | histone (H4) | 329 |
| AT5G60390.3 | STNLDWYK | aminoacyl-tRNA binding | 330 |
| | | factor (eEF1A) | |
| AT5G60390.3 | EHALLAFTLGVK | aminoacyl-tRNA binding | 331 |
| | | factor (eEF1A) | |
| AT5G60390.3 | YYCTVIDAPGHR | aminoacyl-tRNA binding | 332 |
| | | factor (eEF1A) | |
| AT5G60390.3 | NMITGTSQADCAVLIIDSTTGGFEAGISK | aminoacyl-tRNA binding | 333 |
| | | factor (eEF1A) | |
| AT5G61410.2 | VIEAGANALVAGSAVFGAK | phosphopentose epimerase | 334 |
| AT5G64040.1 | CGSNVFWK | component PsaN of PS-I | 335 |
| | | complex | |
| AT5G64040.2 | FPENFTGCQDLAK | component PsaN of PS-I | 336 |
| | | complex | |
| AT5G66140.1 | ALLEVVESGGK | component alpha type-4 of | 337 |
| | | 26S proteasome | |
| AT5G66190.2 | LDFAVSR | ferredoxin-NADP | 338 |
| | | oxidoreductase | |

### Example 4

### Empirical Identification of Conserved Peptides in Vascular Plants

An empirical mass spectrometric approach was used to identify conserved peptides in pineapple (Ananas comosus), Thale Cress (Arabidopsis thaliana ), Flooded gum (Eucalyptus grandis), bean (Phaseolus vulgaris), native yam (Dioscorea transversa), elkhorn fern (Platycerium bifurcatum), burrawang (Macrozamia communis), loblolly pine (Pinus taeda), tomato (Solanum lycopersicum), waratah (Telopea speciosissima), grape (Vitis Vinifera), and maize (Zea mays). The 12 species were selected to span the diversity of vascular plants (see FIG. 5).

Briefly, an ion library (SWATH library) was created for Arabidopsis, based on mass spectrometric data from three Arabidopsis leaf samples. Lys-C and trypsin digested protein extracts from the three leaf samples were analyzed on a Sciex 6600 TripleTOF mass spectrometer with a data dependent acquisition method according to Aspinwall et al. (2019), “Range size and growth temperature influence Eucalyptus species responses to an experimental heatwave,” Glob. Chang. Biol. 25:1665-1684. The resulting data were matched to a list of Arabidopsis proteins (available at the arabidopsis.org website, TAIR10) using ProteinPilot (Sciex). The ProteinPilot.group file was used to create a SWATH library in the PeakView SWATH microapp (Sciex) with a peptide FDR of <1%.

The same Arabidopsis samples, and three samples each from the 11 additional species (pineapple, flooded gum, bean, native yam, elkhorn fern, burrawang, loblolly pine, tomato, waratah, grape, and maize) were analyzed using data independent SWATH (Aspinwall et al., 2019). The MS data from this analysis were matched to the Arabidopsis ion library using the SWATH microapp, identifying conserved peptides across the 12 different species and ensuring that the peptides were observable through MS analysis. Merely using an amino acid sequence alignment approach may produce peptides that may not be reliably observed through MS analysis. Presence/absence of conserved peptides were based on FDR scores assigned by the SWATH microapp, i.e., a peptide was considered genuinely present in a species, and conserved between that species and Arabidopsis, if all three replicates from a species had a peptide FDR <1%.

A subset of 105 conserved peptides (see Table 4 below) was selected to be used as a set of isotope labeled internal standards for absolute quantification of their corresponding proteins in subsequent analyses of leaves from additional plant species. Most of the selected peptides were present in all 12 of the diverse species, meaning that they are likely present in all vascular plants. Additional criteria for selection included standard chemical stability preferences for isotope labeled peptide standards, such as peptides not arising from unfavorable trypsin cleavage sites and not containing amino acids likely to undergo spontaneous chemical modification (based on Pratt et al. 2006, “Multiplexed absolute quantification for proteomics using concatenated signature peptides encoded by QconCAT genes,” Nat. Protoc. 1:1029-43). Peptides were also selected so that highly conserved protein complexes were represented, e.g., PSII, ATP synthase. The stoichiometries of protein subunits within conserved complexes are themselves often highly conserved. Therefore, amounts of overall complexes can be inferred from isotope labeled standards covering a small number of subunits within the complex.

TABLE 4
Subset of 105 conserved peptides

| | | | Exemplary | | |
|---|---|---|---|---|---|
| | | | TAIR10 or | | SEQ |
| QconCAT | | Protein | Uniprot | MapMan protein | ID |
| number | Peptide | target | protein | description | NO: |
| 1 | LIFQYASFNNSR | psbA/D1 | atcg00020 | component PsbA/D1 of | 339 |
| | | | | PS-II reaction center | |
| | | | | complex | |
| 1 | VINTWADIINR | psbA/D1 | atcg00020 | component PsbA/D1 of | 340 |
| | | | | PS-II reaction center | |
| | | | | complex | |
| 1 | AYDFVSQEIR | psbD/D2 | atcg00270 | component PsbD/D2 of | 341 |
| | | | | PS-II reaction center | |
| | | | | complex | |
| 1 | NILLNEGIR | psbD/D2 | atcg00270 | component PsbD/D2 of | 342 |
| | | | | PS-II reaction center | |
| | | | | complex | |
| 1 | LAFYDYIGNNPAK | psbB/CP47 | atcg00680 | component PsbB/CP47 | 343 |
| | | | | of PS-II reaction center | |
| | | | | complex | |
| 1 | VHTVVLNDPGR | psbB/CP47 | atcg00680 | component PsbB/CP47 | 344 |
| | | | | of PS-II reaction center | |
| | | | | complex | |
| 1 | APWLEPLR | psbC/CP43 | atcg00280 | component PsbC/CP43 | 345 |
| | | | | of PS-II reaction center | |
| | | | | complex | |
| 1 | DQETTGFAWWAGNAR | psbC/CP43 | atcg00280 | component PsbC/CP43 | 346 |
| | | | | of PS-II reaction center | |
| | | | | complex | |
| 1 | YPIYVGGNR | petA | atcg00540 | apocytochrome f | 347 |
| | | | | component PetA of | |
| | | | | cytochrome b6/f | |
| | | | | complex | |
| 1 | VYDWFEER | petB | atcg00720 | apocytochrome b | 348 |
| | | | | component PetB of | |
| | | | | cytochrome b6/f | |
| | | | | complex | |
| 1 | DFGYSFPC[Pye]DGPGR | psaB | atcg00340 | apoprotein PsaB of PS- | 349 |
| | | | | I complex | |
| 1 | DKPVALSIVQAR | psaB | atcg00340 | apoprotein PsaB of PS- | 350 |
| | | | | I complex | |
| 1 | QILIEPIFAQWIQSAHGK | psaB | atcg00340 | apoprotein PsaB of PS- | 351 |
| | | | | I complex | |
| 1 | VFPNGEVQYLHPK | PsaD | at4g02770 | component PsaD of PS- | 352 |
| | | | | I complex | |
| 1 | FVQAGSEVSALLGR | atpB | atcg00480 | subunit beta of | 353 |
| | | | | peripheral CF1 | |
| | | | | subcomplex of ATP | |
| | | | | synthase complex | |
| 1 | LSIFETGIK | atpB | atcg00480 | subunit beta of | 354 |
| | | | | peripheral CF1 | |
| | | | | subcomplex of ATP | |
| | | | | synthase complex | |
| 1 | DTDILAAFR | RbcL | atcg00490 | large subunit of | 355 |
| | | | | ribulose-1,5- | |
| | | | | bisphosphat | |
| | | | | carboxylase/oxygenase | |
| | | | | heterodimer | |
| 1 | TFQGPPHGIQVER | RbcL | atcg00490 | large subunit of | 356 |
| | | | | ribulose-1,5- | |
| | | | | bisphosphat | |
| | | | | carboxylase/oxygenase | |
| | | | | heterodimer | |
| 1 | FYWAPTR | RCA | at2g39730 | ATP-dependent | 357 |
| | | | | activase involved in | |
| | | | | RuBisCo regulation | |
| 1 | VYDDEVR | RCA | at2g39730 | ATP-dependent | 358 |
| | | | | activase involved in | |
| | | | | RuBisCo regulation | |
| 1 | IGVIESLLEK | PGK | at3g12780 | phosphoglycerate | 359 |
| | | chloroplast | | kinase | |
| 1 | AAALNIVPTSTGAAK | GAPB | at1g42970 | glyceraldehyde 3- | 360 |
| | | | | phosphate | |
| | | | | dehydrogenase | |
| 1 | VIITAPAK | GAPB | at1g42970 | glyceraldehyde 3- | 361 |
| | | | | phosphate | |
| | | | | dehydrogenase | |
| 1 | GKRLASIGLENTEANR | FBA1 | at2g21330 | fructose 1,6- | 362 |
| | | | | bisphosphate aldolase | |
| 1 | YIGSLVGDFHR | CFBP1 | at3G54050 | fructose-1,6- | 363 |
| | | | | bisphosphatase | |
| 1 | FFQLYVYK | GLO1, | at3g14420 | glycolate oxidase | 364 |
| | | GOX1 | | | |
| 1 | NFEGLDLGK | GLO1, | at3g14420 | glycolate oxidase | 365 |
| | | GOX1 | | | |
| 1 | AIPWIFAWTQTR | PEPC2 | at2g42600 | PEP carboxylase | 366 |
| 1 | AIPWIFSWTQTR | PEPC | This variant of PEPC is not in | | 367 |
| | | mutant | Arabidopsis, but it is in many species | | |
| | | | that undergo C4 photosynthesis. | | |
| 1 | EFAPSIPEK | MDH | at1g04410 | NAD-dependent malate | 368 |
| | | | | dehydrogenase | |
| 1 | VLVVANPANTNALILK | MDH | at1g04410 | NAD-dependent malate | 369 |
| | | | | dehydrogenase | |
| 1 | AGLQFPVGR | Histone | at1g54690 | histone | 370 |
| | | H2A | | | |
| 1 | IFLENVIR | Histone H4 | at5g59970 | histone | 371 |
| 1 | VTGGEVGAASSLAPK | Ribosome | at3g53430 | component RPL12 of | 372 |
| | | LSU | | LSU proteome | |
| | | | | component | |
| 1 | VSGVSLLALFK | Ribosome | at5g02960 | component RPS23 of | 373 |
| | | RPS23 | | SSU proteome | |
| 1 | ELAEDGYSGVEVR | Ribosome | at3g53870 | component RPS3 of | 374 |
| | | RPS3 | | SSU proteome | |
| 1 | GLDVIQQAQSGTGK | EIF4A-2 | at1g54270 | mRNA unwinding | 375 |
| | | | | factor | |
| 1 | VLITTDLLAR | EIF4A-2 | at1g54270 | mRNA unwinding | 376 |
| | | | | factor | |
| 1 | IGGIGTVPVGR | eEF1A | at5g60390 | aminoacyl-tRNA | 377 |
| | | | | binding factor | |
| 1 | LPLQDVYK | eEF1A | at5g60390 | aminoacyl-tRNA | 378 |
| | | | | binding factor | |
| 1 | GSGFVAVEIPFTPR | ClpC1 | at5g50920 | chaperone component | 379 |
| | | | | ClpC of chloroplast | |
| | | | | Clp-type protease | |
| | | | | complex | |
| 1 | TAIAEGLAQR | ClpC1 | at5g50920 | chaperone component | 380 |
| | | | | ClpC of chloroplast | |
| | | | | Clp-type protease | |
| | | | | complex | |
| 1 | GILAADESTGTIGK | FBA8 | at3g52930 | aldolase | 381 |
| 1 | AVDSLVPIGR | Mitochondrial | at2g07698 | subunit alpha of ATP | 382 |
| | | ATP | | synthase peripheral | |
| | | synthase | | MF1 subcomplex | |
| | | alpha | | | |
| 1 | AHGGFSVFAGVGER | Mitochondrial | at5g08680 | subunit beta of ATP | 383 |
| | | ATP | | synthase peripheral | |
| | | synthase | | MF1 subcomplex | |
| | | beta | | | |
| 1 | VVDLLAPYQR | Mitochondrial | at5g08680 | subunit beta of ATP | 384 |
| | | ATP | | synthase peripheral | |
| | | synthase | | MF1 subcomplex | |
| | | beta | | | |
| 1 | AGFAGDDAPR | Actin | at5g09810 | actin filament protein | 385 |
| 1 | IWHHTFYNELR | Actin | at5g09810 | actin filament protein | 386 |
| 1 | ATAGDTHLGGEDFDNR | HSP70-1 | at5g02500 | chaperone | 387 |
| 1 | IINEPTAAAIAYGLDK | HSP70-1 | at5g02500 | chaperone | 388 |
| 1 | ETDGYFIK | ADG1 | at5g48300 | ADP-glucose | 389 |
| | | | | pyrophosphorylase | |
| 1 | IYVLTQFNSASLNR | ADG1 | at5g48300 | ADP-glucose | 390 |
| | | | | pyrophosphorylase | |
| 1 | YNQLLR | Enolase | at2g36530 | Bifunctional enolase | 391 |
| | | | | 2/transcriptional | |
| | | | | activator | |
| | | | | OS = Arabidopsis | |
| | | | | thaliana | |
| 1 | LFTGHPETLEK | Myoglobin, | Uniprot | | 392 |
| | | horse | P68082 | | |
| | | | MYG_HORSE | | |
| 1 | VEADIAGHGQEVLIR | Myoglobin, | Uniprot | | 393 |
| | | horse | P68082 | | |
| | | | MYG_HORSE | | |
| 1 | DEDTQAMPFR | Ovalbumin, | Uniprot | | 394 |
| | | chicken | P01012 | | |
| | | | OVAL_CHICK | | |
| 1 | GGLEPINFQTAADQAR | Ovalbumin, | Uniprot | | 395 |
| | | chicken | P01012 | | |
| | | | OVAL_CHICK | | |
| 1 | ISQAVHAAHAEINEAGR | Ovalbumin, | Uniprot | | 396 |
| | | chicken | P01012 | | |
| | | | OVAL_CHICK | | |
| 2 | WAMLGALGCVFPELLAR | Lhcb1.3 | at1g29930 | component LHCb1/2/3 | 397 |
| | | | | of LHC-II complex | |
| 2 | STPQSIWYGPDRPK | Lhcb2 | at2g05070 | component LHCb1/2/3 | 398 |
| | | | | of LHC-II complex | |
| 2 | ALEVIHGR | Lhcb3 | at5g54270 | component LHCb1/2/3 | 399 |
| | | | | of LHC-II complex | |
| 2 | ECELIHGR | Lhcb4/CP29 | at2g40100 | component LHCb4 of | 400 |
| | | | | LHC-II complex | |
| 2 | LHPGGPFDPLGLAK | Lhcb5/CP26 | at4g10340 | component LHCb5 of | 401 |
| | | | | LHC-II complex | |
| 2 | TGALLLDGNTLNYFGK | Lhcb5/CP26 | at4g10340 | component LHCb5 of | 402 |
| | | | | LHC-II complex | |
| 2 | EAELIHGR | Lhcb6 | at1g15820 | component LHCb6 of | 403 |
| | | | | LHC-II complex | |
| 2 | GGSTGYDNAVALPAGGR | PsbO2 | at3g50820 | component | 404 |
| | | | | PsbO/OEC33 of PS-II | |
| | | | | oxygen-evolving center | |
| 2 | GSSFLDPK | PsbO2 | at3g50820 | component | 405 |
| | | | | PsbO/OEC33 of PS-II | |
| | | | | oxygen-evolving center | |
| 2 | AYGEAANVFGKPK | PsbP | at1g06680 | component PsbP of PS- | 406 |
| | | | | II oxygen-evolving | |
| | | | | center | |
| 2 | AWPYVQNDLR | PsbQ | at4g05180 | component PsbQ of | 407 |
| | | | | PS-II oxygen-evolving | |
| | | | | center | |
| 2 | ANELFVGR | PsbS | at1g44575 | non-photochemical | 408 |
| | | | | quenching PsbS protein | |
| 2 | ESELIHCR | Lhca1 | at3g54890 | component LHCa1 of | 409 |
| | | | | LHC-I complex | |
| 2 | QYFLGLEK | Lhca3 | at1g61520 | component LHCa3 of | 410 |
| | | | | LHC-I complex | |
| 2 | EIPLPHEFILNR | psaA | atcg00350 | apoprotein PsaA of PS- | 411 |
| | | | | I complex | |
| 2 | TAVNPLLR | PsaL | at4g12800 | component PsaL of PS- | 412 |
| | | | | I complex | |
| 2 | VYLWHETTR | PsaC | atcg01060 | component PsaC of PS- | 413 |
| | | | | I complex | |
| 2 | EIIIDVPLASR | PsaF | at1g31330 | component PsaF of PS- | 414 |
| | | | | I complex | |
| 2 | LYSIASSAIGDFGDSK | FNR | at5g66190 | ferredoxin-NADP | 415 |
| | | | | oxidoreductase | |
| 2 | GYISPYFVTDSEK | Cnp60 | at1g55490 | subunit beta of Cpn60 | 416 |
| | | | | chaperonin complex | |
| 2 | LADLVGVTLGPK | Cnp60 | at1g55490 | subunit beta of Cpn60 | 417 |
| | | | | chaperonin complex | |
| 2 | AMHAVIDR | RbcL | atcg00490 | large subunit of | 418 |
| | | | | ribulose-1,5-bisphosphat | |
| | | | | carboxylase/oxygenase | |
| | | | | heterodimer | |
| 2 | SQAETGEIK | RbcL | atcg00490 | large subunit of | 419 |
| | | | | ribulose-1,5- | |
| | | | | bisphosphat | |
| | | | | carboxylase/oxygenase | |
| | | | | heterodimer | |
| 2 | LDELIYVESHLSNLSTK | PRK | at1g32060 | phosphoribulokinase | 420 |
| 2 | QYADAVIEVLPTTLIPD | PRK | at1g32060 | phosphoribulokinase | 421 |
| | DNEGK | | | | |
| 2 | GVTTIIGGGDSVAAVEK | PGK both | at1g56190 | phosphoglycerate | 422 |
| | | | | kinase | |
| 2 | GGAFTGEISVEQLK | TIM | at2g21170 | triosephosphate | 423 |
| | | | | isomerase | |
| 2 | EAAWGLAR | FBA1 | at2g21330 | fructose 1,6- | 424 |
| | | | | bisphosphate aldolase | |
| 2 | VTTTIGYGSPNK | TKL1 | at3g60750 | transketolase | 425 |
| 2 | YTGGMVPDVNQIIVK | SBPase | at3g55800 | sedoheptulose-1,7- | 426 |
| | | | | bisphosphatase | |
| 2 | IDLAIDGADEVDPNLDLVK | RPI3 | at3g04790 | phosphopentose | 427 |
| | | | | isomerase | |
| 2 | LVFVTNNSTK | PGLP1B | at5g36790 | phosphoglycolate | 428 |
| | | | | phosphatase | |
| 2 | LLEATGISTVPGSGFGQK | GGT1 | at1g23310 | glutamate-glyoxylate | 429 |
| | | | | transaminase | |
| 2 | LAVEAWGLK | AGT1 | at2g13360 | serine-glyoxylate | 430 |
| | | | | transaminase | |
| 2 | IAILNANYMAK | GLDP1 | at4g33010 | glycine dehydrogenase | 431 |
| | | | | component P-protein of | |
| | | | | glycine cleavage | |
| | | | | system | |
| 2 | SLLALQGPLAAPVLQHLTK | GDCST | at1g11860 | aminomethyltransferase | 432 |
| | | | | component T-protein | |
| | | | | of glycine cleavage | |
| | | | | system | |
| 2 | YSEGYPGAR | SHM1 | at4g37930 | serine | 433 |
| | | | | hydroxymethyltransferase | |
| 2 | GQTVGVIGAGR | HPR | at1g68010 | hydroxypyruvate | 434 |
| | | | | reductase | |
| 2 | FDFDPLDVTK | catalase | at1g20620 | catalase | 435 |
| 2 | FSVSPVVR | eEF2 | at1g56070 | mRNA-translocation | 436 |
| | | | | factor | |
| 2 | GVQYLNEIK | eEF2 | at1g56070 | mRNA-translocation | 437 |
| | | | | factor | |
| 2 | AASFNIIPSSTGAAK | GAPC2 | at1g13440 | NAD-dependent | 438 |
| | | | | glyceraldehyde 3- | |
| | | | | phosphate | |
| | | | | dehydrogenase | |
| 2 | VPTVDVSVVDLTVR | GAPC2 | at1g13440 | NAD-dependent | 439 |
| | | | | glyceraldehyde 3-phosphate | |
| | | | | dehydrogenase | |
| 2 | LVAGLPEGGVLLLENVR | PGK | at1g79550 | phosphoglycerate | 440 |
| | | | | kinase | |
| 2 | LAADTPLLTGQR | Vacuolar | at1g78900 | subunit A of V-type | 441 |
| | | ATP | | ATPase peripheral V1 | |
| | | synthase A | | subcomplex | |
| 2 | AVVQVFEGTSGIDNK | Vacuolar | at1g76030 | subunit B of V-type | 442 |
| | | ATP | | ATPase peripheral V1 | |
| | | synthase B | | subcomplex | |
| 2 | AILNLSLR | GS2 | at5g35630 | plastidial glutamine | 443 |
| | | | | synthetase | |
| 2 | EHIAAYGEGNER | GSR1 | at5g37600 | cytosolic glutamine | 444 |
| | | | | synthetase | |
| 2 | LVAEAGIGTVASGVAK | GLU1 | at5g04140 | Fd-dependent | 445 |
| | | | | glutamate synthase | |
| 2 | VCPSHILNFQPGEAFVVR | BCA | at3g01500 | | 446 |
| 2 | DVATILHWK | BCA | at3g01500 | | 447 |
| 2 | FALESFWDGK | ATCIMS | at5g17920 | methyl- | 448 |
| | | | | tetrahydrofolate- | |
| | | | | dependent methionine | |
| | | | | synthase | |
| 2 | DEDTQAMPFR | Ovalbumin, | Uniprot | | 449 |
| | | chicken | P01012 | | |
| | | | OVAL_CHICK | | |
| 2 | GGLEPINFQTAADQAR | Ovalbumin, | Uniprot | | 450 |
| | | chicken | P01012 | | |
| | | | OVAL_CHICK | | |
| 2 | VEADIAGHGQEVLIR | Myoglobin, | Uniprot | | 451 |
| | | horse | P68082 | | |
| | | | MYG_HORSE | | |
| 1 | MAGRNFEGLDLGKELA | | Full | | 452 |
| | EDGYSGVEVRAHGGFS | | QconCAT1 | | |
| | VFAGVGERTAIAEGLA | | amino acid | | |
| | QREFAPSIPEKGGLEPIN | | sequence | | |
| | FQTAADQARLPLQDVY | | | | |
| | KAYDFVSQEIRGKRLAS | | | | |
| | IGLENTEANRDKPVALS | | | | |
| | IVQARAGFAGDDAPRQI | | | | |
| | LIEPIFAQWIQSAHGKIG | | | | |
| | GIGTVPVGRVHTVVLN | | | | |
| | DPGRVYDDEVRLSIFET | | | | |
| | GIKVYDWFEERLIFQYA | | | | |
| | SFNNSRVSGVSLLALFK | | | | |
| | ETDGYFIKVIITAPAKYP | | | | |
| | IYVGGNRAVDSLVPIGR | | | | |
| | AGLQFPVGRVVDLLAP | | | | |
| | YQRLAFYDYIGNNPAK | | | | |
| | VLVVANPANTNALILK | | | | |
| | AIPWIFAWTQTRLFTGH | | | | |
| | PETLEKFVQAGSEVSAL | | | | |
| | LGRNILLNEGIRFYWAP | | | | |
| | TRGLDVIQQAQSGTGK | | | | |
| | ATAGDTHLGGEDFDNR | | | | |
| | DFGYSFPCDGPGRAAA | | | | |
| | LNIVPTSTGAAKISQAV | | | | |
| | HAAHAEINEAGRYIGSL | | | | |
| | VGDFHRYNQLLRIGVIE | | | | |
| | SLLEKFFQLYVYKVLIT | | | | |
| | TDLLARIYVLTQFNSAS | | | | |
| | LNRAPWLEPLRGILAA | | | | |
| | DESTGTIGKIWHHTFYN | | | | |
| | ELRVTGGEVGAASSLA | | | | |
| | PKVFPNGEVQYLHPKVI | | | | |
| | NTWADIINRIFLENVIRII | | | | |
| | NEPTAAAIAYGLDKTF | | | | |
| | QGPPHGIQVERGSGFVA | | | | |
| | VEIPFTPRDQETTGFAW | | | | |
| | WAGNARVEADIAGHG | | | | |
| | QEVLIRAIPWIFSWTQT | | | | |
| | RDTDILAAFRDEDTQA | | | | |
| | MPFRLAAALEHHHHHH | | | | |
| 2 | HMAGRGGLEPINFQTA | | Full | | 453 |
| | ADQARLHPGGPFDPLG | | QconCAT2 | | |
| | LAKTGALLLDGNTLNY | | amino acid | | |
| | FGKDEDTQAMPFRWA | | sequence | | |
| | MLGALGCVFPELLARA | | | | |
| | WPYVQNDLRYSEGYPG | | | | |
| | ARFSVSPVVRGVQYLN | | | | |
| | EIKEAELIHGRECELIHG | | | | |
| | RAYGEAANVFGKPKAN | | | | |
| | ELFVGRLVFVTNNSTKL | | | | |
| | LEATGISTVPGSGFGQK | | | | |
| | LAVEAWGLKQYFLGLE | | | | |
| | KESELIHCREIIIDVPLAS | | | | |
| | RVYLWHETTREIPLPHE | | | | |
| | FILNRTAVNPLLRSTPQ | | | | |
| | SIWYGPDRPKAILNLSL | | | | |
| | RIAILNANYMAKSLLAL | | | | |
| | QGPLAAPVLQHLTKGQ | | | | |
| | TVGVIGAGRAMHAVID | | | | |
| | REHIAAYGEGNERALE | | | | |
| | VIHGRGVTTIIGGGDSV | | | | |
| | AAVEKGGAFTGEISVE | | | | |
| | QLKEAAWGLARGGST | | | | |
| | GYDNAVALPAGGRFAL | | | | |
| | ESFWDGKFDFDPLDVT | | | | |
| | KLYSIASSAIGDFGDSK | | | | |
| | GSSFLDPKLVAEAGIGT | | | | |
| | VASGVAKSQAETGEIKI | | | | |
| | DLAIDGADEVDPNLDL | | | | |
| | VKLDELIYVESHLSNLS | | | | |
| | TKQYADAVIEVLPTTLI | | | | |
| | PDDNEGKLADLVGVTL | | | | |
| | GPKGYISPYFVTDSEKY | | | | |
| | TGGMVPDVNQIIVKVT | | | | |
| | TTIGYGSPNKAVVQVFE | | | | |
| | GTSGIDNKLAADTPLLT | | | | |
| | GQRLVAGLPEGGVLLL | | | | |
| | ENVRVPTVDVSVVDLT | | | | |
| | VRAASFNIIPSSTGAAK | | | | |
| | DVATILHWKVCPSHILN | | | | |
| | FQPGEAFVVRVEADIA | | | | |
| | GHGQEVLIRLAAALEH | | | | |
| | HHHHH | | | | |

Enzymatic and biological functions of the proteins targeted by the isotope labeled peptides were assigned using the MapMan functional annotation scheme (Schwacke et al., 2019). The MapMan scheme arranges protein functions hierarchically, including the subunits of complexes. Additionally, the stoichiometries of protein complex subunits were determined from publicly available sources, for example from crystallography and electron microscopy data (e.g., the RCSB Protein Data Bank, available at the rcsb.org website).

Exemplary processes for protein quantification using conserved peptides are set out in the further Examples below.

### Example 5

### Protein Quantification in Leaves of Three Plant Species

The conserved peptides identified in Example 4 were made into QconCATs by PolyQuant (Germany). The full sequences of the QconCATs are set out in Table 4 (SEQ ID Nos: 452 and 453). QconCAT1 contained 15N and 13C labeled lysines and arginines. QconCAT2 lysines are arginines were labeled with only 13C. The cysteines in both QconCATs were alkylated for 1 hour with 2-vinylpyridine in N-methylmorpholine/acetic acid buffer; reactions were stopped with 2-mercaptoethanol. The alkylated QconCATs were combined into a stock solution at equimolar concentrations, approximately 50 ng/μL of each.

Leaf Sample Protein Extraction

Leaf protein extraction from three species (Flooded gum, bean, corn) was carried out via the methods described in Aspinwall et al. (2019). Critically, the extraction method is quantitative and extracts nearly all the protein from leaves. Also, the leaf area of each sample was known and 38 picomoles of ovalbumin per square centimeter of leaf was added to each sample early in the extraction protocol as an internal standard. Ovalbumin was used instead of QconCATs early in the protocol because it is far less expensive. QconCATs were added later in the protocol to a small proportion of the overall extracted leaf protein. Adding QconCATs to samples early in the protocol instead of ovalbumin is functionally equivalent to adding ovalbumin early and QconCATs later. The QconCATs both contained ovalbumin peptides, which allowed measured target-to-standard ratios to be converted to target per leaf area based on the addition rate of ovalbumin (38 μmol cm⁻²). Additionally, target protein amounts per leaf dry weight can be calculated if dry weight per leaf area is known.

Addition of QconCAT to the Leaf Samples, Acetate Solvent Protein Extraction Method and Lys-C/trypsin Digestion

Following the alkylation step in the leaf protein extraction method, extract protein concentrations were measured using a FluroProfile Protein Quantification Kit (Sigma). Then 50 μg protein was transferred to a new microcentrifuge tube and combined with 10 μg of the QconCAT stock solution (˜0.5 μg each QconCAT). The mixture was then subjected to a methanol-chloroform extraction method modified to be quantitative according to Aspinwall et al. (2019). The resulting pellets were digested with Lys-C and trypsin in a mass spec-compatible N-methylmorpholine buffer containing Rapigest detergent (Waters) according to Aspinwall et al. (2019), with modifications to promote complete digestion. Modifications included a higher concentration of trypsin, 1.25 μg per digest, and the addition of 4 mM CaCl₂. Lys-C digestion at 45° C. for 1 hour was followed by the addition of trypsin and an overnight incubation at 37° C. Digests were stopped by the addition of 2% TFA.

If peptides are chemically synthesized instead of produced as QconCATs, then the peptides are added to samples following trypsin digestion. Also, QconCATs can be digested separately from samples and added as peptides following the digestion step as if they were chemically synthesized peptides. The addition of peptides post-digestion works with or without ovalbumin as an internal standard added during the extraction method. However, adding ovalbumin or intact QconCATs early in the extraction method is preferable to adding only peptides post-digestion because the added proteins effectively account for non-specific protein losses during sample processing.

Mass Spectrometric Analysis

Following digestion, the peptides were subjected to mass spectrometric analysis according to

Aspinwall et al. (2019). Briefly, 0.2 μg peptides per sample were analyzed by SWATH LC-MS/MS on a Sciex TripleTOF 6600 according to Cain et al. (2019) with the following modifications. The column was 10 centimeters and was run at room temperature. The acquisition LC gradient was 60 minutes. Sixty (60) variable width SWATH windows were used.

Using SWATH to analyze samples that include isotope labeled standards differs from more typical targeted mass spectrometry methods such as Selected Reaction Monitoring (SRM). SRM sets the mass spectrometer to only measure targeted analytes and their corresponding internal standards. SWATH captures data for all observable peptides in a sample—afterwards, data for the target analytes and internal standards are extracted using software. SWATH data allow the analysis of additional proteins not represented by internal standards by other means, if desired, without having to re-run the sample on a mass spectrometer.

SWATH Data Analysis

SWATH data were analyzed using MultiQuant software (Sciex), which extracts and integrates chromatograms for individual target peptide fragment ions. A list of target fragment ions, four per peptide for each target peptide and four for each isotope labeled standard, was created manually and used for the MultiQuant integration method. Example target peptide fragment ions (transitions) are shown in Table 5. The data in Table 5 can be used to create a Selected Reaction Monitoring method to target peptides with a mass spectrometer method, as opposed to extracting those data from SWATH results. The resulting outputs, integrated peak areas for each fragment ion of interest, were exported to Excel.

TABLE 5
Sample target peptide fragment ions (transitions)

| | | QconCAT | Retention | precursor | fragment |
|---|---|---|---|---|---|
| protein_name | peptide | # | time | m/z | m/z |
| GAPB | AAALNIVPTSTGAAK | 1 | 20.8 | 692.8934 | 732.3887 |
| GAPB | AAALNIVPTSTGAAK | 1 | 20.8 | 692.8934 | 831.457 |
| GAPB | AAALNIVPTSTGAAK | 1 | 20.8 | 692.8934 | 1058.584 |
| GAPB | AAALNIVPTSTGAAK | 1 | 20.8 | 692.8934 | 944.5411 |
| GAPB | AAALNIVPTSTGAAK[+08] | 1 | 20.8 | 696.9005 | 740.4028 |
| GAPB | AAALNIVPTSTGAAK[+08] | 1 | 20.8 | 696.9005 | 839.4713 |
| GAPB | AAALNIVPTSTGAAK[+08] | 1 | 20.8 | 696.9005 | 1066.598 |
| GAPB | AAALNIVPTSTGAAK[+08] | 1 | 20.8 | 696.9005 | 952.5553 |
| Actin | AGFAGDDAPR | 1 | 9 | 488.7278 | 630.2842 |
| Actin | AGFAGDDAPR | 1 | 9 | 488.7278 | 701.3213 |
| Actin | AGFAGDDAPR | 1 | 9 | 488.7278 | 458.2358 |
| Actin | AGFAGDDAPR | 1 | 9 | 488.7278 | 573.2627 |
| Actin | AGFAGDDAPR[+10] | 1 | 9 | 493.7319 | 640.2924 |
| Actin | AGFAGDDAPR[+10] | 1 | 9 | 493.7319 | 711.3296 |
| Actin | AGFAGDDAPR[+10] | 1 | 9 | 493.7319 | 468.244 |
| Actin | AGFAGDDAPR[+10] | 1 | 9 | 493.7319 | 583.271 |
| Histone H2A | AGLQFPVGR | 1 | 23.7 | 472.7693 | 575.33 |
| Histone H2A | AGLQFPVGR | 1 | 23.7 | 472.7693 | 428.2616 |
| Histone H2A | AGLQFPVGR | 1 | 23.7 | 472.7693 | 703.3886 |
| Histone H2A | AGLQFPVGR | 1 | 23.7 | 472.7693 | 352.1979 |
| Histone H2A | AGLQFPVGR[+10] | 1 | 23.7 | 477.7734 | 585.3383 |
| Histone H2A | AGLQFPVGR[+10] | 1 | 23.7 | 477.7734 | 438.2699 |
| Histone H2A | AGLQFPVGR[+10] | 1 | 23.7 | 477.7734 | 713.3969 |
| Histone H2A | AGLQFPVGR[+10] | 1 | 23.7 | 477.7734 | 357.2021 |

Data Analysis Workflow

Target:standard ratios were calculated for each pair of unlabeled:labeled ions, then the ratios were averaged for each peptide, producing a ratio of moles of target per moles of QconCAT. Those ratios were converted to moles of target protein per cm² using ion areas from unlabeled ovalbumin (added on a per leaf area basis during protein extraction) and the corresponding ovalbumin peptides in the QconCATs. For target proteins that are not part of conserved complexes (e.g., the complexes below), the amounts of protein in grams per leaf area were calculated by multiplying moles by the molecular weight of the corresponding Arabidopsis reference protein. Arabadopsis protein molecular weights are used for all plant species because the structural annotation of Arabidopsis is better than most species and molecular weights of homologs are likely largely conserved. Functional annotations were assigned based on the reference Arabidopsis proteins in the MapMan functional annotation scheme (available at the MapMen Site of Analysis website).

For proteins that are subunits of complexes with highly conserved stoichiometry (e.g., the photosystems, ATP synthase, ribosomes, histones, etc.), the molar ratios of those proteins per complex were calculated from publicly available data such as the RCSB Protein Data Bank. Additional protein subunits in the complexes were also identified in the MapMan scheme from publicly available data, thereby identifying what subunits are effectively quantified by peptides in the QconCATs because they are all part of the same complex with known stoichiometry (shown in Table 7 below). The peptides in the QconCATs include subunits in 25 reference complexes, which, by extension through known complex stoichiometries, covers 167 total complex subunits. Gram amounts of complexes per leaf area were calculated based on the molecular weights of the complexes from publicly available sources.

Results

Amounts of proteins and protein complexes in nanomoles per m² leaf area, plus or minus one standard deviation, for leaf samples from Flooded gum, Bean, and Corn, are shown in Table 6 below. These three species are all examples from the 12 training species used to identify conserved peptides. Samples were extracted and analyzed in triplicate, splitting one leaf into three samples, to demonstrate the technical precision of the method. The average percentage coefficients of variation for Flooded gum, Bean, and Corn were 10%, 9%, and 11%, respectively.

TABLE 6
Amounts of proteins and protein complexes in nmoles per m² leaf area from leaf
samples from flooded gum, bean, and corn

| | | | Flooded | | |
|---|---|---|---|---|---|
| MapMan | | Protein or | gum, nmol | Bean, nmol | Corn, nmol |
| bin | MapMan name | complex | per m² | per m² | per m² |
| 1.1.1.2.1 | Photosynthesis.photophos- | PSII | 1217 ± 168 | 587 ± 32 | 936 ± 104 |
| | phorylation.photosystem | complex | | | |
| | II.PS-II complex.reaction | | | | |
| | center complex | | | | |
| 1.1.1.5.1.2.1 | Photosynthesis.photophos- | PsbS | 881 ± 92 | 482 ± 35 | 34 ± 0 |
| | phorylation.photosystem | | | | |
| | II.photoprotection.non- | | | | |
| | photochemical quenching | | | | |
| | (NPQ).PsbS-dependent | | | | |
| | machinery.regulatory | | | | |
| | protein (PsbS) | | | | |
| 1.1.2 | Photosynthesis.photophos- | Cytochrome | 589 ± 96 | 370 ± 28 | 567 ± 66 |
| | phorylation.cytochrome b6/f | b6/f | | | |
| | complex | | | | |
| 1.1.4.2 | Photosynthesis.photophos- | PSI | 524 ± 87 | 190 ± 27 | 357 ± 47 |
| | phorylation.photosystem | complex | | | |
| | I.PS-I complex | | | | |
| 1.1.5.2.1 | Photosynthesis.photophos- | FNR | 22 ± 3 | 273 ± 15 | 89 ± 10 |
| | phorylation.linear electron | | | | |
| | flow.ferredoxin-NADP | | | | |
| | reductase (FNR) | | | | |
| | activity.ferredoxin-NADP | | | | |
| | oxidoreductase | | | | |
| 1.1.8.1.6.2 | Photosynthesis.photophos- | Cnp60 | 42 ± 3 | 60 ± 3 | 36 ± 4 |
| | phorylation.chlororespiration. | complex | | | |
| | NADH dehydrogenase- | | | | |
| | like (NDH) | | | | |
| | complex.assembly and | | | | |
| | stabilization.Cpn60 | | | | |
| | chaperonin heterodimer | | | | |
| 1.1.9 | Photosynthesis.photophos- | ATP | 438 ± 38 | 325 ± 17 | 638 ± 70 |
| | phorylation.ATP synthase | synthase | | | |
| | complex | complex | | | |
| 1.2.1.1 | Photosynthesis.calvin | Rubisco | 3733 ± 433 | 3476 ± 223 | 1129 ± 128 |
| | cycle.ribulose-1,5- | complex | | | |
| | bisphosphat | | | | |
| | carboxylase/oxygenase | | | | |
| | (RuBisCo) | | | | |
| | activity.RuBisCo | | | | |
| | heterodimer | | | | |
| 1.2.1.2.1 | Photosynthesis.calvin | Cnp60 | 42 ± 3 | 60 ± 3 | 36 ± 4 |
| | cycle.ribulose-1,5- | complex | | | |
| | bisphosphat | | | | |
| | carboxylase/oxygenase | | | | |
| | (RuBisCo) | | | | |
| | activity.RuBisCo | | | | |
| | assembly.CPN60 assembly | | | | |
| | chaperone complex | | | | |
| 1.2.1.3.2 | Photosynthesis.calvin | RCA | 2803 ± 89 | 2891 ± 170 | 563 ± 70 |
| | cycle.ribulose-1,5- | | | | |
| | bisphosphat | | | | |
| | carboxylase/oxygenase | | | | |
| | (RuBisCo) | | | | |
| | activity.RuBisCo | | | | |
| | regulation.ATP-dependent | | | | |
| | activase (RCA) | | | | |
| 1.2.2 | Photosynthesis.calvin | PGK both | 84 ± 6 | 540 ± 23 | 1071 ± 149 |
| | cycle.phosphoglycerate | | | | |
| | kinase | | | | |
| 1.2.2 | Photosynthesis.calvin | PGK | 569 ± 92 | 513 ± 229 | 1316 ± 176 |
| | cycle.phosphoglycerate | chloroplast | | | |
| | kinase | | | | |
| 1.2.3 | Photosynthesis.calvin | GAP | 254 ± 24 | 156 ± 7 | 365 ± 42 |
| | cycle.glyceraldehyde 3- | | | | |
| | phosphate dehydrogenase | | | | |
| 1.2.5 | Photosynthesis.calvin | FBA | 1347 ± 62 | 937 ± 63 | 2320 ± 230 |
| | cycle.fructose 1,6- | chloroplast | | | |
| | bisphosphate aldolase | | | | |
| 1.2.6 | Photosynthesis.calvin | FBPase | 271 ± 46 | 137 ± 8 | 268 ± 32 |
| | cycle.fructose-1,6- | | | | |
| | bisphosphatase | | | | |
| 1.2.7 | Photosynthesis.calvin | Transketolase | 459 ± 40 | 351 ± 18 | 6 ± 1 |
| | cycle.transketolase | | | | |
| 1.2.8 | Photosynthesis.calvin | SBPase | 376 ± 28 | 252 ± 10 | 359 ± 36 |
| | cycle.sedoheptulose-1,7- | | | | |
| | bisphosphatase | | | | |
| 1.3.1 | Photosynthesis.photo- | PGLP | 147 ± 18 | 100 ± 5 | 36 ± 3 |
| | respiration.phosphoglycolate | | | | |
| | phosphatase | | | | |
| 1.3.2 | Photosynthesis.photo- | GLO | 246 ± 33 | 611 ± 295 | 123 ± 15 |
| | respiration.glycolate oxidase | | | | |
| 1.3.3.1 | Photosynthesis.photo- | GGT | 242 ± 20 | 169 ± 10 | 58 ± 6 |
| | respiration.aminotransferase | | | | |
| | activities.glutamate- | | | | |
| | glyoxylate transaminase | | | | |
| 1.3.3.2 | Photosynthesis.photo- | AGT | 551 ± 40 | 250 ± 13 | 8 ± 0 |
| | respiration.aminotransferase | | | | |
| | activities.serine-glyoxylate | | | | |
| | transaminase | | | | |
| 1.3.4.1 | Photosynthesis.photo- | GLDP | 1180 ± 290 | 350 ± 13 | 66 ± 14 |
| | respiration.glycine decarboxylase | | | | |
| | complex.glycine | | | | |
| | dehydrogenase component | | | | |
| | P-protein | | | | |
| 1.3.4.2 | Photosynthesis.photo- | GDCST | 493 ± 33 | 157 ± 7 | 5 ± 1 |
| | respiration.glycine decarboxylase | | | | |
| | complex.aminomethyltrans- | | | | |
| | ferase component T-protein | | | | |
| 1.3.5 | Photosynthesis.photo- | SHM | 425 ± 15 | 225 ± 11 | 44 ± 3 |
| | respiration.serine | | | | |
| | hydroxymethyltransferase | | | | |
| | (SHM) | | | | |
| 1.3.6 | Photosynthesis.photo- | HPR | 172 ± 5 | 103 ± 11 | 38 ± 5 |
| | respiration.hydroxypyruvate | | | | |
| | reductase (HPR) | | | | |
| 1.4.1.1 | Photosynthesis.CAM/C4 | PEPC | 73 ± 3 | 53 ± 2 | 2829 ± 350 |
| | photosynthesis.phosphoenol- | | | | |
| | pyruvate (PEP) | | | | |
| | carboxylase activity.PEP | | | | |
| | carboxylase | | | | |
| 1.4.2 | Photosynthesis.CAM/C4 | MDH | 150 ± 15 | 95 ± 7 | 196 ± 19 |
| | photosynthesis.NAD- | | | | |
| | dependent malate | | | | |
| | dehydrogenase | | | | |
| 2.1.1.2 | Cellular | FBA8 | 338 ± 31 | 186 ± 13 | 99 ± 11 |
| | respiration.glycolysis.cytosolic | | | | |
| | glycolysis.aldolase | | | | |
| 2.1.1.4.1 | Cellular | GAPC2 | 305 ± 12 | 183 ± 6 | 616 ± 80 |
| | respiration.glycolysis.cytosolic | | | | |
| | glycolysis.glyceraldehyde | | | | |
| | 3-phosphate dehydrogenase | | | | |
| | activities .NAD-dependent | | | | |
| | glyceraldehyde 3- | | | | |
| | phosphate dehydrogenase | | | | |
| 2.4.6 | Cellular | ATP | 78 ± 6 | 31 ± 2 | 45 ± 2 |
| | respiration.oxidative | synthase | | | |
| | phosphorylation.ATP | mitochondrial | | | |
| | synthase complex | | | | |
| 3.1.2.2 | Carbohydrate | FBA8 | 338 ± 31 | 186 ± 13 | 99 ± 11 |
| | metabolism.sucrose | | | | |
| | metabolism.biosynthesis.cytosolic | | | | |
| | fructose- | | | | |
| | bisphosphate aldolase | | | | |
| 3.2.2.3 | Carbohydrate | ADG1 | 151 ± 23 | 82 ± 4 | 130 ± 13 |
| | metabolism, starch | | | | |
| | metabolism.biosynthesis.ADP- | | | | |
| | glucose | | | | |
| | pyrophosphorylase | | | | |
| 3.9.2.3 | Carbohydrate | Transketolase | 459 ± 40 | 351 ± 18 | 6 ± 1 |
| | metabolism.oxidative | | | | |
| | pentose phosphate | | | | |
| | pathway.non-oxidative | | | | |
| | phase.transketolase | | | | |
| 3.12.2 | Carbohydrate | FBA | 1347 ± 62 | 937 ± 63 | 2320 ± 230 |
| | metabolism.plastidial | chloroplast | | | |
| | glycolysis.fructose-1,6- | | | | |
| | bisphosphate aldolase | | | | |
| 3.12.5 | Carbohydrate | PGK both | 84 ± 6 | 540 ± 23 | 1071 ± 149 |
| | metabolism.plastidial | | | | |
| | glycolysis.phosphoglycerate | | | | |
| | kinase | | | | |
| 3.12.5 | Carbohydrate | PGK | 569 ± 92 | 513 ± 229 | 1316 ± 176 |
| | metabolism.plastidial | chloroplast | | | |
| | glycolysis.phosphoglycerate | | | | |
| | kinase | | | | |
| 4.1.2.1.3 | Amino acid | AGT | 551 ± 40 | 250 ± 13 | 8 ± 0 |
| | metabolism.biosynthesis. | | | | |
| | aspartate | | | | |
| | family.asparagine.asparagine | | | | |
| | aminotransaminase | | | | |
| 4.1.2.2.6.2.1 | Amino acid | ATCIMS | 22 ± 3 | 39 ± 3 | 50 ± 8 |
| | metabolism.biosynthesis. | | | | |
| | aspartate family.aspartate- | | | | |
| | derived amino | | | | |
| | acids.methionine.L- | | | | |
| | homocysteine S- | | | | |
| | methyltransferase | | | | |
| | activities.methyl- | | | | |
| | tetrahydrofolate-dependent | | | | |
| | methionine synthase | | | | |
| 5.1.1.3 | Lipid metabolism.fatty acid | MDH | 150 ± 15 | 95 ± 7 | 196 ± 19 |
| | biosynthesis.citrate | | | | |
| | shuttle.cytosolic NAD- | | | | |
| | dependent malate | | | | |
| | dehydrogenase | | | | |
| 10.2.1 | Redox | Catalase | 116 ± 50 | 132 ± 75 | 9 ± 1 |
| | homeostasis.enzymatic | | | | |
| | reactive oxygen species | | | | |
| | scavengers.catalase | | | | |
| 12.1 | Chromatin | Histone | 169 ± 17 | 53 ± 5 | 218 ± 26 |
| | organisation.histones | complex | | | |
| 17.1.2 | Protein | Ribosome | 104 ± 9 | 74 ± 8 | 102 ± 11 |
| | biosynthesis.ribosome | complex | | | |
| | biogenesis.large ribosomal | | | | |
| | subunit (LSU) | | | | |
| 17.4.2 | Protein | EIF4 | 128 ± 12 | 54 ± 7 | 87 ± 8 |
| | biosynthesis.translation | | | | |
| | initiation.mRNA loading | | | | |
| 17.5.1.1 | Protein | eEF1A | 559 ± 40 | 295 ± 18 | 553 ± 79 |
| | biosynthesis.translation | | | | |
| | elongation.eEF1 | | | | |
| | aminoacyl-tRNA binding | | | | |
| | factor activity.aminoacyl- | | | | |
| | tRNA binding factor | | | | |
| | (eEF1A) | | | | |
| 17.5.2.1 | Protein | eEF2 | 97 ± 2 | 57 ± 1 | 99 ± 11 |
| | biosynthesis.translation | | | | |
| | elongation.eEF2 mRNA- | | | | |
| | translocation factor | | | | |
| | activity.mRNA- | | | | |
| | translocation factor (eEF2) | | | | |
| 18.4.25.2 | Protein | PGLP | 147 ± 18 | 100 ± 5 | 36 ± 3 |
| | modification.phosphorylation. | | | | |
| | aspartate-based protein | | | | |
| | phosphatase | | | | |
| | superfamily.phosphatase | | | | |
| | (CIN) | | | | |
| 19.1.5.1 | Protein homeostasis.protein | HSP70-1 | 300 ± 10 | 124 ± 8 | 161 ± 18 |
| | quality control.cytosolic | | | | |
| | Hsp70 chaperone | | | | |
| | system.chaperone (Hsp70) | | | | |
| 19.1.7 | Protein homeostasis.protein | Cnp60 | 42 ± 3 | 60 ± 3 | 36 ± 4 |
| | quality control.Hsp60 | complex | | | |
| | chaperone system | | | | |
| 19.4.2.9.4 | Protein | ClpC1 | 112 ± 12 | 83 ± 3 | 100 ± 9 |
| | homeostasis.proteolysis.serine- | | | | |
| | type peptidase | | | | |
| | activities.chloroplast Clp- | | | | |
| | type protease | | | | |
| | complex.chaperone | | | | |
| | component ClpC | | | | |
| 20.2.1 | Cytoskeleton | Actin | 194 ± 23 | 132 ± 8 | 166 ± 15 |
| | organisation.microfilament | | | | |
| | network.actin filament | | | | |
| | protein | | | | |
| 24.1.1 | Solute transport.primary | ATP | 13 ± 1 | 10 ± 0 | 14 ± 2 |
| | active transport.V-type | synthase | | | |
| | ATPase complex | vacuolar | | | |
| 25.1.5.1.1 | Nutrient uptake.nitrogen | GSR1 | 785 ± 72 | 20 ± 3 | 110 ± 15 |
| | assimilation.ammonium | | | | |
| | assimilation.glutamine | | | | |
| | synthetase | | | | |
| | activities.cytosolic | | | | |
| | glutamine synthetase | | | | |
| | (GLN1) | | | | |
| 25.1.5.1.2 | Nutrient uptake.nitrogen | GS2 | 1268 ± 288 | 1375 ± 91 | 268 ± 68 |
| | assimilation.ammonium | | | | |
| | assimilation.glutamine | | | | |
| | synthetase | | | | |
| | activities.plastidial | | | | |
| | glutamine synthetase | | | | |
| | (GLN2) | | | | |
| 25.1.5.2.1 | Nutrient uptake.nitrogen | GLU1 | 130 ± 18 | 98 ± 4 | 6 ± 0 |
| | assimilation.ammonium | | | | |
| | assimilation.glutamate | | | | |
| | synthase activities.Fd- | | | | |
| | dependent glutamate | | | | |
| | synthase | | | | |
| 50.4.2 | Enzyme | Enolase | 236 ± 15 | 99 ± 7 | 186 ± 18 |
| | classification.EC_4 | | | | |
| | lyases.EC_4.2 carbon- | | | | |
| | oxygen lyase | | | | |

TABLE 7
Complexes quantified in Examples 5 and 6

| | | | Subunit | | | | | | Number |
|---|---|---|---|---|---|---|---|---|---|
| | | | MapMan | | Reference | Reference | Complex | | of gene |
| | | Complex | bins in | | subunit | subunit | reference | | products |
| | Complex | MapMan | the entire | Reference | MapMan | copies per | subunit | Complex | in |
| Complex | abbreviation | bin | complex | subunits | bin | complex | ratio | MW | complex |
| Photosystem | PSII | 1.1.1.2 | 1.1.1.2.1 | atcg00020.1, | 1.1.1.2.1.1, | 1, 1, 1, 1 | 1 | 331496 | 22 |
| II | | | to | atcg00270.1, | 1.1.1.2.1.2, | | | | |
| | | | 1.1.1.2.2. | atcg00680.1, | 1.1.1.2.1.3, | | | | |
| | | | 2.2; | atcg00280.1 | 1.1.1.2.1.4 | | | | |
| | | | 1.1.1.2.3 | | | | | | |
| | | | to | | | | | | |
| | | | 1.1.1.2.15 | | | | | | |
| Cytochrome | b6f | 1.1.2 | 1.1.2.1 to | atcg00540.1, | 1.1.2.1, | 1, 1 | 1 | 106448 | 8 |
| b6f | | | 1.1.2.8 | atcg00720.1 | 1.1.2.2 | | | | |
| Photosystem | PSI | 1.1.4.2 | 1.1.4.2.1 | atcg00350.1, | 1.1.4.2.1, | 1, 1 | 1 | 298740 | 14 |
| I | | | to | atcg00340.1 | 1.1.4.2.2 | | | | |
| | | | 1.1.4.2.12, | | | | | | |
| | | | 1.1.4.2.14 | | | | | | |
| Chloroplast | Cnp60 | 1.1.8.1.6.1 | 1.1.8.1.6.1.1, | at1g55490.2 | 1.1.8.1.6.1.2 | 3 | 0.333333 | 822645 | 3 |
| chaperonin | | | 1.1.8.1.6.1.2 | | | | | | |
| Cnp60 | | | | | | | | | |
| ATP | ATP | 1.1.9 | 1.1.9.1 to | atcg00480.1 | 1.1.9.2.2 | 3 | 0.333333 | 569743 | 9 |
| synthase | synthase | | 1.1.9.2.5 | | | | | | |
| chloroplastic | chloroplastic | | | | | | | | |
| Rubisco | Rubisco | 1.2.1.1 | 1.2.1.1.1, | atcg00490.1 | 1.2.1.1.1 | 8 | 0.125 | 541468 | 2 |
| | | | 1.2.1.1.2 | | | | | | |
| Chloroplastic | GAP | 1.2.3 | 1.2.3 | at1g42970.1, | 1.2.3 | 4 | 0.25 | 152622 | 1 |
| glyceraldehyde | chloroplast | | | at3g26650.1, | | | | | |
| 3- | | | | at1g12900.4 | | | | | |
| phosphate | | | | | | | | | |
| dehydrogenase | | | | | | | | | |
| Cytosolic | GAP | 2.1.4.1 | 2.1.4.1 | at1g13440 | 2.1.4.1 | 4 | 0.25 | 147657 | 1 |
| glyceraldehyde | cytosolic | | | | | | | | |
| 3- | | | | | | | | | |
| phosphate | | | | | | | | | |
| dehydrogenase | | | | | | | | | |
| Mitochondrial | Mitochondrial | 2.5.6 | 2.5.6.1 to | at2g07698.1, | 2.5.6.2.1, | 3, 3 | 0.333333 | 604886 | 13 |
| ATP | ATP | | 2.5.6.2.6 | at5g08680.1 | 2.5.6.2.2 | | | | |
| synthase | synthase | | | | | | | | |
| ADP- | ADG | 3.2.1 | 3.2.1.3 | at5g48300.1 | 3.2.1.3 | 2 | 0.5 | 202388 | 2 |
| glucose | | | | | | | | | |
| pyrophosph | | | | | | | | | |
| orylase | | | | | | | | | |
| Histones | Histones | 12.1 | 12.1.1 to | at1g54690.1, | 12.1.2, | 2, 2 | 0.5 | 144073 | 5 |
| | | | 12.1.5 | at5g59970.1 | 12.1.5 | | | | |
| Cytosolic | Ribosome | 17.1 | 17.1.1 to | at3g53430.1, | 17.1.1.1.12, | 1, 1 | 1 | 1330626 | 71 |
| ribosome | | | 17.1.2.1. | at5g02960.1 | 17.1.2.1.24 | | | | |
| | | | 33 | | | | | | |
| Eukaryotic | EIF4A | 17.3.2.1 | 17.3.2.1, | at3g13920.1 | 17.3.2.1 | 1 | 1 | 261013 | 3 |
| initiation | | | 17.3.2.3.1, | | | | | | |
| factor-4A | | | 17.3.2.3.2 | | | | | | |
| Vacuolar | Vacuolar | 24.2.1 | 24.2.1 to | at1g78900.2, | 24.2.1.2.1, | 3, 3 | 0.333333 | 797895 | 13 |
| ATP | ATP | | 24.2.1.2.8 | at1g76030.1 | 24.2.1.2.2 | | | | |
| synthase | synthase | | | | | | | | |

| | |
|---|---|
| 25 reference subunits | 167 |

### Example 6

### Measurement of Leaf Proteins for Two Species Outside the Training Set of 12 Vascular Plant Species

Two species, Cotton (Gossypium hirsutum) and Myoporum montanum, not in the training set used to identify conserved plant proteins, and not in orders represented in the training set, were analyzed using the methods in Example 5. The species were analyzed in triplicate, one leaf sample per plant from three plants. Table 8 below shows the protein and complex in mg per m² leaf area included in addition to nmoles per m² leaf area. The average percentage coefficient of variation for cotton and Myoporum were 28% and 12%, respectively. The larger CVs than the species in Example 5 may reflect biological variation across the triplicate plants.

TABLE 8
Protein and complex in mg per m² leaf area

| | | | | | Myoporum | Myoporum |
|---|---|---|---|---|---|---|
| | | | | | montanum, | montanum, |
| MapMan | | Protein or | Cotton, nmol | Cotton, mg | nmol per | mg per |
| bin | MapMan name | complex | per m² | per m² | m² | m² |
| 1.1.1.2.1 | Photosynthesis.photophos- | PSII | 771 ± | 255.5 ± | 1906 ± | 631.8 ± |
| | phorylation.photosystem II.PS-II | complex | 104 | 34.6 | 202 | 67.1 |
| | complex.reaction center complex | | | | | |
| 1.1.1.5.1.2.1 | Photosynthesis.photophos- | PsbS | 449 ± | 9.7 ± | 1858 ± 76 | 40.1 ± 1.6 |
| | phorylation.photosystem | | 114 | 2.5 | | |
| | II.photoprotection.non- | | | | | |
| | photochemical quenching | | | | | |
| | (NPQ).PsbS-dependent | | | | | |
| | machinery.regulatory protein | | | | | |
| | (PsbS) | | | | | |
| 1.1.2 | Photosynthesis.photophosphorylation. | Cytochrome | 466 ± | 49.6 ± | 702 ± 111 | 74.7 ± |
| | cytochrome b6/f complex | b6/f | 229 | 24.3 | | 11.8 |
| 1.1.4.2 | Photosynthesis.photophosphorylation. | PSI | 427 ± | 127.4 ± | 770 ± 150 | 230 ± 44.9 |
| | photosystem I.PS-I complex | complex | 5 | 1.6 | | |
| 1.1.5.2.1 | Photosynthesis.photophosphorylation. | FNR | 6 ± 1 | 0.2 ± 0 | 774 ± 108 | 27.2 ± 3.8 |
| | linear electron flow.ferredoxin- | | | | | |
| | NADP reductase (FNR) | | | | | |
| | activity.ferredoxin-NADP | | | | | |
| | oxidoreductase | | | | | |
| 1.1.8.1.6.2 | Photosynthesis.photophosphorylation. | Cnp60 | 42 ± | 34.9 ± | 68 ± 7 | 55.7 ± 5.4 |
| | chlororespiration.NADH | complex | 23 | 18.7 | | |
| | dehydrogenase-like (NDH) | | | | | |
| | complex.assembly and | | | | | |
| | stabilization.Cpn60 chaperonin | | | | | |
| | heterodimer | | | | | |
| 1.1.9 | Photosynthesis.photophosphorylation. | ATP | 307 ± | 174.9 ± | 718 ± 84 | 408.9 ± |
| | ATP synthase complex | synthase | 92 | 52.3 | | 48.1 |
| | | complex | | | | |
| 1.2.1.1 | Photosynthesis.calvin | Rubisco | 3442 ± | 1863.9 ± | 10012 ± | 5420.9 ± |
| | cycle.ribulose-1,5-bisphosphat | complex | 1184 | 641.4 | 592 | 320.5 |
| | carboxylase/oxygenase (RuBisCo) | | | | | |
| | activity.RuBisCo heterodimer | | | | | |
| 1.2.1.2.1 | Photosynthesis.calvin | Cnp60 | 42 ± | 34.9 ± | 68 ± 7 | 55.7 ± 5.4 |
| | cycle.ribulose-1,5-bisphosphat | complex | 23 | 18.7 | | |
| | carboxylase/oxygenase (RuBisCo) | | | | | |
| | activity.RuBisCo assembly.CPN60 | | | | | |
| | assembly chaperone complex | | | | | |
| 1.2.1.3.2 | Photosynthesis.calvin | RCA | 2637 ± | 122 ± | 3654 ± | 169.1 ± |
| | cycle.ribulose-1,5-bisphosphat | | 927 | 42.9 | 863 | 39.9 |
| | carboxylase/oxygenase (RuBisCo) | | | | | |
| | activity.RuBisCo regulation.ATP- | | | | | |
| | dependent activase (RCA) | | | | | |
| 1.2.2 | Photosynthesis.calvin | PGK both | 470 ± | 20.1 ± | 1347 ± | 57.4 ± 5.1 |
| | cycle.phosphoglycerate kinase | | 160 | 6.8 | 120 | |
| 1.2.2 | Photosynthesis.calvin | PGK | 456 ± | 19.4 ± | 2947 ± | 125.7 ± |
| | cycle.phosphoglycerate kinase | chloroplast | 139 | 5.9 | 487 | 20.8 |
| 1.2.3 | Photosynthesis.calvin | GAP | 175 ± | 26.7 ± | 384 ± 38 | 58.6 ± 5.7 |
| | cycle.glyceraldehyde 3-phosphate | | 70 | 10.7 | | |
| | dehydrogenase | | | | | |
| 1.2.5 | Photosynthesis.calvin | FBA | 912 ± | 34.7 ± | 3736 ± | 142 ± 7.1 |
| | cycle.fructose 1,6-bisphosphate | chloroplast | 189 | 7.2 | 187 | |
| | aldolase | | | | | |
| 1.2.6 | Photosynthesis.calvin | FBPase | 111 ± | 4.3 ± 1 | 482 ± 47 | 18.8 ± 1.8 |
| | cycle.fructose-1,6-bisphosphatase | | 25 | | | |
| 1.2.7 | Photosynthesis.calvin | Transketolase | 288 ± | 21 ± | 29 ± 15 | 2.1 ± 1.1 |
| | cycle.transketolase | | 89 | 6.5 | | |
| 1.2.8 | Photosynthesis.calvin | SBPase | 211 ± | 7.3 ± | 520 ± 45 | 18 ± 1.6 |
| | cycle.sedoheptulose-1,7- | | 56 | 1.9 | | |
| | bisphosphatase | | | | | |
| 1.3.1 | Photosynthesis.photorespiration. | PGLP | 109 ± | 3.7 ± | 267 ± 12 | 9.1 ± 0.4 |
| | phosphoglycolate phosphatase | | 41 | 1.4 | | |
| 1.3.2 | Photosynthesis.photorespiration. | GLO | 468 ± | 18.9 ± | 2179 ± | 87.9 ± |
| | glycolate oxidase | | 92 | 3.7 | 839 | 33.8 |
| 1.3.3.1 | Photosynthesis.photorespiration. | GGT | 264 ± | 14.1 ± | 524 ± 65 | 27.9 ± 3.5 |
| | aminotransferase | | 92 | 4.9 | | |
| | activities.glutamate-glyoxylate | | | | | |
| | transaminase | | | | | |
| 1.3.3.2 | Photosynthesis.photorespiration. | AGT | 413 ± | 18.3 ± | 1057 ± 92 | 46.7 ± 4 |
| | aminotransferase activities.serine- | | 87 | 3.8 | | |
| | glyoxylate transaminase | | | | | |
| 1.3.4.1 | Photosynthesis.photorespiration. | GLDP | 542 ± | 57 ± | 1661 ± | 174.8 ± |
| | glycine decarboxylase | | 242 | 25.4 | 317 | 33.3 |
| | complex.glycine dehydrogenase | | | | | |
| | component P-protein | | | | | |
| 1.3.4.2 | Photosynthesis.photorespiration. | GDCST | 248 ± | 10.3 ± | 488 ± 25 | 20.4 ± 1.1 |
| | glycine decarboxylase | | 44 | 1.8 | | |
| | complex.aminomethyltransferase | | | | | |
| | component T-protein | | | | | |
| 1.3.5 | Photosynthesis.photorespiration. | SHM | 236 ± | 12.8 ± | 1180 ± 81 | 63.7 ± 4.4 |
| | serine hydroxymethyltransferase | | 54 | 2.9 | | |
| | (SHM) | | | | | |
| 1.3.6 | Photosynthesis.photorespiration. | HPR | 104 ± | 4.4 ± | 506 ± 41 | 21.4 ± 1.7 |
| | hydroxypyruvate reductase (HPR) | | 22 | 0.9 | | |
| 1.4.1.1 | Photosynthesis.CAM/C4 | PEPC | 40 ± 9 | 4.4 ± 1 | 144 ± 17 | 15.8 ± 1.8 |
| | photosynthesis.phosphoenolpyruvate | | | | | |
| | (PEP) carboxylase activity.PEP | | | | | |
| | carboxylase | | | | | |
| 1.4.2 | Photosynthesis.CAM/C4 | MDH | 56 ± | 2 ± 0.4 | 366 ± 17 | 13 ± 0.6 |
| | photosynthesis.NAD-dependent | | 11 | | | |
| | malate dehydrogenase | | | | | |
| 2.1.1.2 | Cellular | FBA8 | 193 ± | 7.4 ± | 950 ± 13 | 36.5 ± 0.5 |
| | respiration.glycolysis.cytosolic | | 70 | 2.7 | | |
| | glycolysis.aldolase | | | | | |
| 2.1.1.4.1 | Cellular | GAPC2 | 198 ± | 29.3 ± | 694 ± 83 | 102.5 ± |
| | respiration.glycolysis.cytosolic | | 52 | 7.7 | | 12.2 |
| | glycolysis.glyceraldehyde 3- | | | | | |
| | phosphate dehydrogenase | | | | | |
| | activities.NAD-dependent | | | | | |
| | glyceraldehyde 3-phosphate | | | | | |
| | dehydrogenase | | | | | |
| 2.4.6 | Cellular respiration.oxidative | ATP | 28 ± 2 | 16.7 ± | 118 ± 9 | 71.2 ± 5.6 |
| | phosphorylation.ATP synthase | synthase | | 1.3 | | |
| | complex | mitochondrial | | | | |
| 3.1.2.2 | Carbohydrate metabolism.sucrose | FBA8 | 193 ± | 7.4 ± | 950 ± 13 | 36.5 ± 0.5 |
| | metabolism.biosynthesis.cytosolic | | 70 | 2.7 | | |
| | fructose-bisphosphate aldolase | | | | | |
| 3.2.2.3 | Carbohydrate metabolism.starch | ADG1 | 100 ± | 20.2 ± | 194 ± 7 | 39.2 ± 1.4 |
| | metabolism.biosynthesis.ADP- | | 45 | 9.1 | | |
| | glucose pyrophosphorylase | | | | | |
| 3.9.2.3 | Carbohydrate | Transketolase | 288 ± | 21 ± | 29 ± 15 | 2.1 ± 1.1 |
| | metabolism.oxidative pentose | | 89 | 6.5 | | |
| | phosphate pathway.non-oxidative | | | | | |
| | phase.transketolase | | | | | |
| 3.12.2 | Carbohydrate | FBA | 912 ± | 34.7 ± | 3736 ± | 142 ± 7.1 |
| | metabolism.plastidial | chloroplast | 189 | 7.2 | 187 | |
| | glycolysis.fructose-1,6- | | | | | |
| | bisphosphate aldolase | | | | | |
| 3.12.5 | Carbohydrate | PGK both | 470 ± | 20.1 ± | 1347 ± | 57.4 ± 5.1 |
| | metabolism.plastidial | | 160 | 6.8 | 120 | |
| | glycolysis.phosphoglycerate | | | | | |
| | kinase | | | | | |
| 3.12.5 | Carbohydrate | PGK | 456 ± | 19.4 ± | 2947 ± | 125.7 ± |
| | metabolism.plastidial | chloroplast | 139 | 5.9 | 487 | 20.8 |
| | glycolysis.phosphoglycerate | | | | | |
| | kinase | | | | | |
| 4.1.2.1.3 | Amino acid | AGT | 413 ± | 18.3 ± | 1057 ± 92 | 46.7 ± 4 |
| | metabolism.biosynthesis.aspartate | | 87 | 3.8 | | |
| | family.asparagine.asparagine | | | | | |
| | aminotransaminase | | | | | |
| 4.1.2.2.6.2.1 | Amino acid | ATCIMS | 3 ± 1 | 0.3 ± 0 | 100 ± 23 | 8.4 ± 1.9 |
| | metabolism.biosynthesis.aspartate | | | | | |
| | family.aspartate-derived amino | | | | | |
| | acids.methionine.L-homocysteine | | | | | |
| | S-methyltransferase | | | | | |
| | activities.methyl-tetrahydrofolate- | | | | | |
| | dependent methionine synthase | | | | | |
| 5.1.1.3 | Lipid metabolism.fatty acid | MDH | 56 ± | 2 ± 0.4 | 366 ± 17 | 13 ± 0.6 |
| | biosynthesis.citrate | | 11 | | | |
| | shuttle.cytosolic NAD-dependent | | | | | |
| | malate dehydrogenase | | | | | |
| 10.2.1 | Redox homeostasis.enzymatic | Catalase | 134 ± | 7.6 ± | 211 ± 35 | 12 ± 2 |
| | reactive oxygen species | | 28 | 1.6 | | |
| | scavengers.catalase | | | | | |
| 12.1 | Chromatin organisation.histones | Histone | 207 ± | 29.8 ± | 836 ± 130 | 120.4 ± |
| | | complex | 29 | 4.2 | | 18.7 |
| 17.1.2 | Protein biosynthesis.ribosome | Ribosome | 89 ± | 118.2 ± | 186 ± 16 | 246.9 ± |
| | biogenesis.large ribosomal subunit | complex | 42 | 56.5 | | 20.7 |
| | (LSU) | | | | | |
| 17.4.2 | Protein biosynthesis.translation | EIF4 | 52 ± 7 | 13.7 ± | 177 ± 2 | 46.3 ± 0.6 |
| | initiation.mRNA loading | | | 1.8 | | |
| 17.5.1.1 | Protein biosynthesis.translation | eEF1A | 370 ± | 18.3 ± | 882 ± 48 | 43.7 ± 2.4 |
| | elongation.eEF1 aminoacyl-tRNA | | 99 | 4.9 | | |
| | binding factor activity.aminoacyl- | | | | | |
| | tRNA binding factor (eEF1A) | | | | | |
| 17.5.2.1 | Protein biosynthesis.translation | eEF2 | 76 ± | 7.1 ± | 151 ± 9 | 14.1 ± 0.9 |
| | elongation.eEF2 mRNA- | | 23 | 2.1 | | |
| | translocation factor | | | | | |
| | activity.mRNA-translocation | | | | | |
| | factor (eEF2) | | | | | |
| 18.4.25.2 | Protein | PGLP | 109 ± | 3.7 ± | 267 ± 12 | 9.1 ± 0.4 |
| | modification.phosphorylation. | | 41 | 1.4 | | |
| | aspartate-based protein phosphatase | | | | | |
| | superfamily.phosphatase (CIN) | | | | | |
| 19.1.5.1 | Protein homeostasis.protein | HSP70-1 | 138 ± | 9.9 ± | 614 ± 116 | 43.7 ± 8.2 |
| | quality control.cytosolic Hsp70 | | 22 | 1.6 | | |
| | chaperone system.chaperone | | | | | |
| | (Hsp70) | | | | | |
| 19.1.7 | Protein homeostasis.protein | Cnp60 | 42 ± | 34.9 ± | 68 ± 7 | 55.7 ± 5.4 |
| | quality control.Hsp60 chaperone | complex | 23 | 18.7 | | |
| | system | | | | | |
| 19.4.2.9.4 | Protein | ClpC1 | 69 ± | 6.9 ± | 232 ± 13 | 23.1 ± 1.3 |
| | homeostasis.proteolysis.serine- | | 23 | 2.3 | | |
| | type peptidase | | | | | |
| | activities.chloroplast Clp-type | | | | | |
| | protease complex.chaperone | | | | | |
| | component ClpC | | | | | |
| 20.2.1 | Cytoskeleton | Actin | 184 ± | 7.7 ± | 416 ± 24 | 17.3 ± 1 |
| | organisation.microfilament | | 53 | 2.2 | | |
| | network.actin filament protein | | | | | |
| 24.1.1 | Solute transport.primary active | ATP | 9 ± 1 | 6.8 ± | 48 ± 2 | 38 ± 1.5 |
| | transport.V-type ATPase complex | synthase | | 0.9 | | |
| | | vacuolar | | | | |
| 25.1.5.1.1 | Nutrient uptake.nitrogen | GSR1 | 83 ± | 3.2 ± | 697 ± 94 | 27.2 ± 3.7 |
| | assimilation.ammonium | | 18 | 0.7 | | |
| | assimilation.glutamine synthetase | | | | | |
| | activities.cytosolic glutamine | | | | | |
| | synthetase (GLN1) | | | | | |
| 25.1.5.1.2 | Nutrient uptake.nitrogen | GS2 | 1012 ± | 43 ± | 2729 ± | 115.9 ± |
| | assimilation.ammonium | | 370 | 15.7 | 481 | 20.4 |
| | assimilation.glutamine synthetase | | | | | |
| | activities.plastidial glutamine | | | | | |
| | synthetase (GLN2) | | | | | |
| 25.1.5.2.1 | Nutrient uptake.nitrogen | GLU1 | 72 ± | 11.8 ± | 351 ± 50 | 58 ± 8.2 |
| | assimilation.ammonium | | 19 | 3.2 | | |
| | assimilation.glutamate synthase | | | | | |
| | activities.Fd-dependent glutamate | | | | | |
| | synthase | | | | | |
| 50.4.2 | Enzyme classification.EC_4 | Enolase | 107 ± | 5.1 ± | 309 ± 25 | 14.8 ± 1.2 |
| | lyases.EC_4.2 carbon-oxygen | | 38 | 1.8 | | |
| | lyase | | | | | |

### Example 7

### Absolute Protein Quantification makes New Types of Biological Insights Possible

This example demonstrates how absolute quantification of proteins and protein complexes across multiple species makes new types of biological comparisons possible. Amounts of key components of photosynthesis across 14 species were compared. The 14 species are the 12 species used in Example 4 and the two species in Example 6.

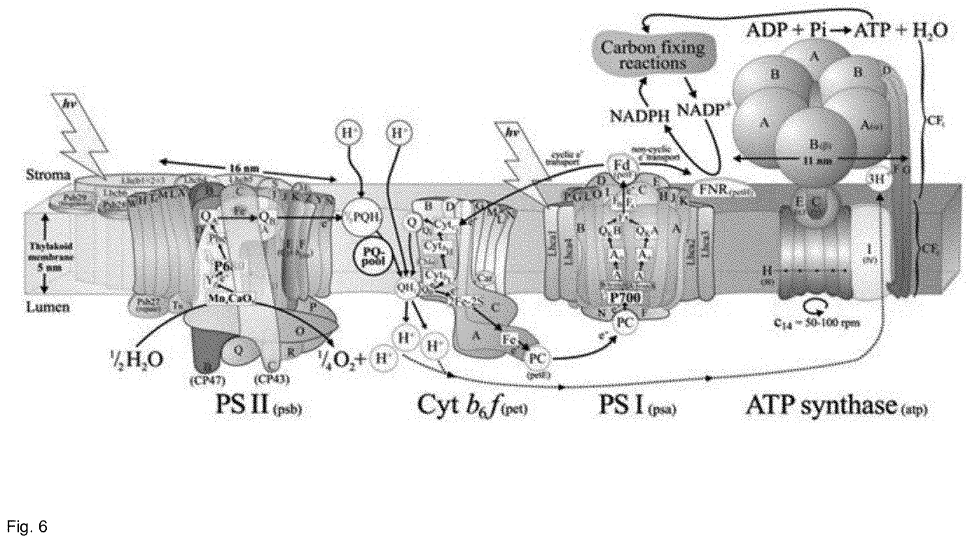

FIG. 6 exemplifies figures of the proteins of photosynthesis found in most university biochemistry and plant physiology textbooks (see Orr and Govindjee (2013), “Photosynthesis Web Resources,” Photosynthesis Research 115:179-214). It shows the major complexes (Photosystems I and II, ATP synthase, Cytochrome b6f) and demonstrates how they are complexes of protein subunits.

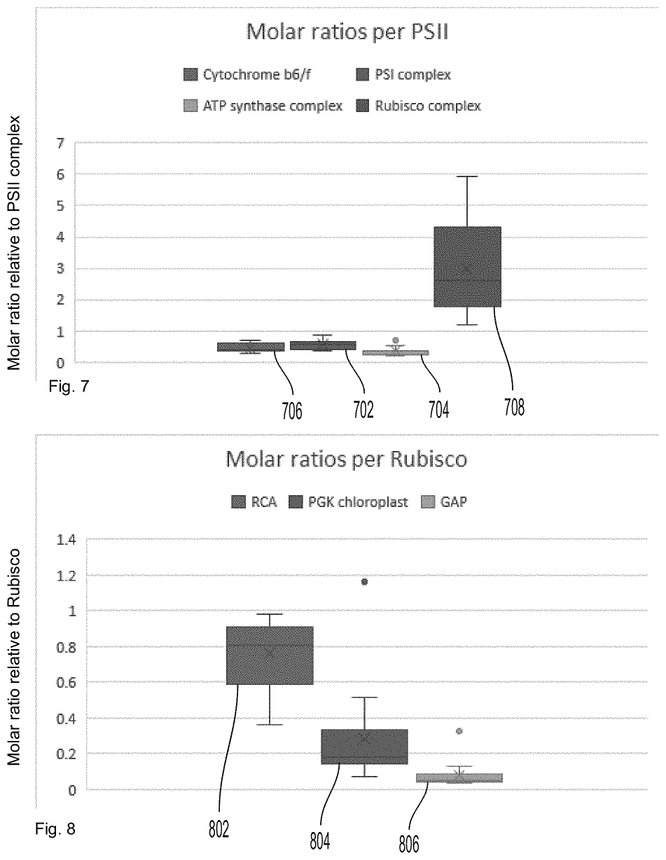

FIG. 7 contains box and whisker plots that summarize the 14 species' protein complex ratios relative to PSII. The ratios of the membrane associated complexes of the light-dependent reactions of photosynthesis, PSI complex (box 702), ATP synthase (box 704), and Cytochrome b6f (box 706), are all conserved with respect to PSII. However, the ratio relative to PSII of Rubisco (box 708), which is not membrane-associated and is part of the light-independent reactions, is not conserved. These sorts of quantitative comparisons across different protein complexes and across species are not possible without isotopically labeled peptide standards that can be used across multiple species.

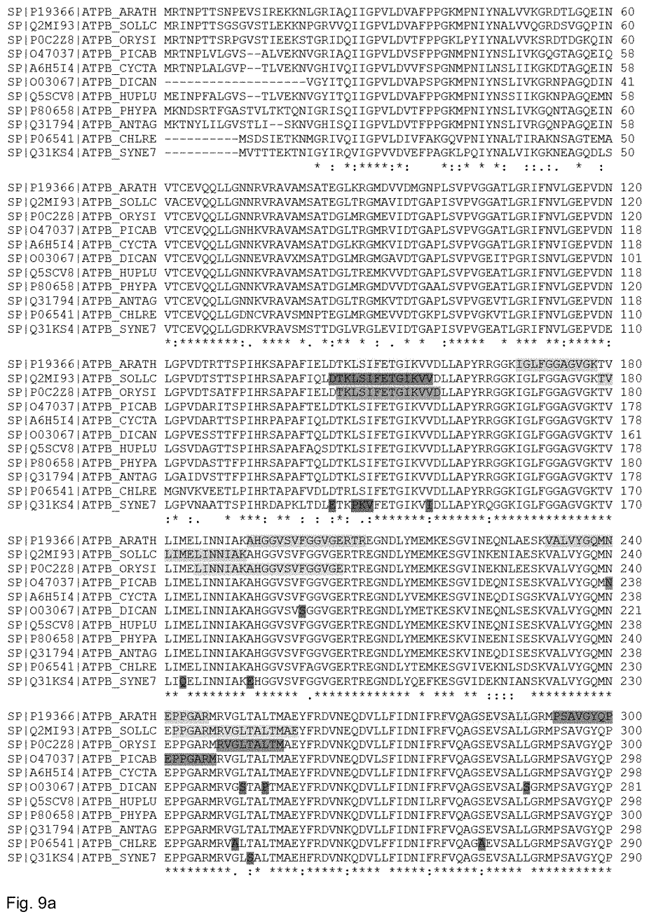

FIG. 8 is a similar box and whisker plot summarizing ratios from the 14 species, but the ratios are relative to Rubisco and the proteins are related to the light-independent reactions of photosynthesis. RCA (box 802) is Rubisco activase, an enzyme that interacts closely with Rubisco to keep Rubisco active during the day. PGK (box 804) and GAP (box 806) are enzymes of the Calvin cycle—the carbon fixing light-independent reactions. FIG. 8 shows that, on a molar basis, there is nearly as much RCA as Rubisco. For PGK and GAP there are outliers with much higher ratios relative to Rubisco. The outliers are both from corn, which probably reflects the different type of photosynthesis corn uses (C4) compared to most other plants (which are C3). C4 plants like corn have mechanisms to enhance the carbon dioxide fixing activity of Rubisco, which means that less Rubisco per amount of other carbon fixing enzymes is required. Like the example in FIG. 7, the quantitative comparisons across proteins and species in FIG. 8 are not possible without internal peptide standards that work across species. Both examples demonstrate how the approach in this disclosure make possible new types of biological insights.

### Example 8

### ATP Synthase Example

A list of 105 conserved tryptic peptides were identified in Example 4 and utilized in Examples 5 through 7. That set of peptides is not exhaustive—there are numerous additional peptides produced by trypsin that could be used as standards. Similarly, additional conserved peptides can be generated by cleavage methods other than trypsin, for example by cyanogen bromide chemical cleavage or cleavage by other proteases such as Asp N. Therefore, the method of using conserved peptides is not restricted to the 105 peptides used in Examples 5 through 7. The invention is extensible to additional cleavage methods, including gas phase fragmentation of intact proteins. In the case of intact protein mass spectrometry, conserved fragment ions could be identified and intact isotope labeled proteins containing those fragment sequences could be used as internal standards.

To demonstrate how different protein digestion and hydrolysis methods produce additional potential conserved peptides, the protein sequences for the beta subunit of chloroplastic ATP synthase from 11 diverse species were aligned. The alignment illustrates stretches of conserved amino acid sequences across the 11 species. Two of the conserved stretches were used in the previous examples to quantify chloroplastic ATP synthase—they are peptides produced by trypsin digestion.

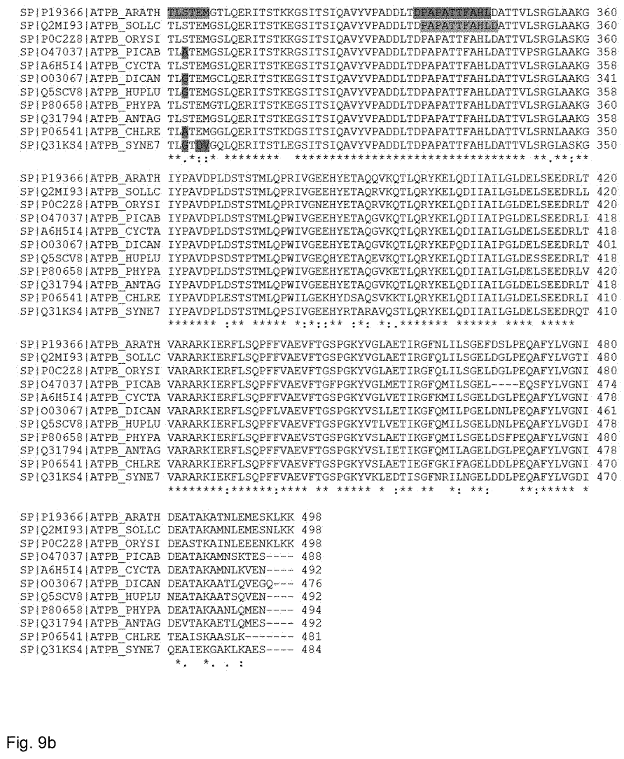

Photosynthetic eukaryote ATP synthase is a highly conserved protein complex located in chloroplast membranes. Other versions of ATP synthase exist in membranes of vacuoles and mitochondria. The 3 different types of ATP synthase are covered by different peptides in the 105 used in Examples 5 through 7, which makes it possible to quantify the three types of complexes independently. The beta subunit is represented in Examples 4 through 7 by two tryptic peptides. The alignment in FIGS. 9A-9B demonstrates that there are many other conserved peptides in the beta subunit that could be used in the kit, e.g., peptides produced by other proteases and chemical cleavage.

The alignment below contains ATP synthase beta subunits sequences from 11 widely divergent species. One of the species is a prokaryote (marine cyanobacteria Synechococcus elongatus), the rest are eukaryotes. The prokaryote does not have organelles (e.g., chloroplast, mitochondria), but it is photosynthetic and its version of ATP synthase beta is still highly conserved with eukaryotic chloroplastic ATP synthase beta. Eukaryotic chloroplasts and the cyanobacteria from which they arose evolutionarily diverged somewhere between 600 million and 2 billion years ago.

TABLE 9
Proteins in the Alignment

| Protein | Uniprot entry | Entry name | Species | Classification |
|---|---|---|---|---|
| ATP Synthase Beta | P19366 | ATPB_ARATH | Arabidopsis | Angiosperm, dicot, |
| subunit, | | | thaliana | Brassicales |
| chloroplastic | | | | |
| ATP Synthase Beta | Q2MI93 | ATPB_SOLLC | Solanum | Angiosperm, dicot, |
| subunit, | | | lycopersicum | Solanales, tomato |
| chloroplastic | | | | |
| ATP Synthase Beta | P0C2Z8 | ATPB_ORYSI | Oryza sativa | Angiosperm, |
| subunit, | | | | monocot, Poales, |
| chloroplastic | | | | rice |
| ATP Synthase Beta | O47037 | ATPB_PICAB | Picea abies | Gymnosperm, |
| subunit, | | | | Norway spruce |
| chloroplastic | | | | |
| ATP Synthase Beta | A6H5I4 | ATPB_CYCTA | Cycas taitungensis | Cycad |
| subunit, | | | | |
| chloroplastic | | | | |
| ATP Synthase Beta | O03067 | ATPB_DICAN | Dicksonia | Australian tree fern |
| subunit, | | | antarctica | |
| chloroplastic | | | | |
| ATP Synthase Beta | Q5SCV8 | ATPB_HUPLU | Huperzia lucidula | Clubmoss |
| subunit, | | | | |
| chloroplastic | | | | |
| ATP Synthase Beta | P80658 | ATPB_PHYPA | Physcomitrella | Moss |
| subunit, | | | patens | |
| chloroplastic | | | | |
| ATP Synthase Beta | Q31794 | ATPB_ANTAG | Anthoceros | Hornwort |
| subunit, | | | angustus | |
| chloroplastic | | | | |
| ATP Synthase Beta | A0A250WRN1 | ATPB_CHLRE | Chlamydomonas | Unicellular algae |
| subunit, | | | reinhardtii | |
| chloroplastic | | | | |
| ATP Synthase Beta | Q31KS4 | ATPB_SYNE7 | Synechococcus | Cyanobacteria |
| subunit | | | elongatus | |

The two kit peptides for ATP synthase beta are highlighted in FIG. 9A as the following sequences within “SP|P19366|ATPB_ARATH”: (1) the “LSIFETGIK” sequence beginning at position 146 (SEQ ID NO: 354), and (2) the “FVQAGSEVSALLGR” sequence beginning at position 278 (SEQ ID NO: 353). Additional, but not exhaustive, examples of conserved peptides produced by trypsin that have not been used in the kit are highlighted as follows: (1) for “SP|P19366|ATPB_ARATH,” the “IGLFGGAGVGK” sequence beginning at position 168 (SEQ ID NO: 55), the “AHGGVSVFGGVGERTR” sequence beginning at position 192 (SEQ ID NO: 454), and the “VALVYGQMNEPPGAR” sequence beginning at position 232 (SEQ ID NO: 455), and (2) for “SP|Q2MI93|ATPB_SOLLC,” the “TVLIMELINNIAK” sequence beginning at position 179 (SEQ ID NO: 456). Examples of conserved peptides produced by Glu C (not in kit) are highlighted as follows: (1) for “SP|POC2Z8|ATPB_ORYSI,” the “LINNIAKAHGGVSVFGGVGE” sequence beginning at position 185 (SEQ ID NO: 457), and (2) for “SP|Q2MI93|ATPB_SOLLC,” the “PPGARMRVGLTALTMAE” sequence beginning at position 242 (SEQ ID NO: 458). Examples of conserved peptides produced by Asp N (not in kit) are highlighted as follows: (1) for “SP|Q2MI93|ATPB_SOLLC,” the “DTKLSIFETGIKVV” sequence beginning at position 143 (SEQ ID NO: 459), and (2) for “SP|P19366|ATPB_ARATH,” the “DPAPATTFAHL” sequence beginning at position 336 (SEQ ID NO: 460). Examples of conserved peptides produced by formic acid cleavage (C terminal side of Asp) are highlighted as follows: (1) for “SP|P0C2Z8|ATPB_ORYSI,” the “TKLSIFETGIKVVD” sequence beginning at position 144 (SEQ ID NO: 461), and (2) for “SP|Q2MI93|ATPB_SOLLC,” the “PAPATTFAHLD” sequence beginning at position 337 (SEQ ID NO: 462). Examples of conserved peptides produced by cyanogen bromide cleavage (C terminal side of M) are highlighted as follows: (1) for “SP|O47037|ATPB_PICAB,” the “NEPPGARM” sequence beginning at position 238 (SEQ ID NO: 463), (2) for “SP|P19366|ATPB_ARATH,” the “PSAVGYQPTLSTEM” sequence beginning at position 293 (SEQ ID NO: 464), and (3) for “SP|P0C2Z8|ATPB_ORYSI,” the “RVGLTALTM” sequence beginning at position 248 (SEQ ID NO: 465). Residues that conflict with highlighted conserved sequences are highlighted as follows: (1) for “SP|Q31KS4|ATPB_SYNE7,” the “E” residue at position 133, the “PKV” sequence beginning at position 136, the “I” residue at position 146, the “Q” residue at position 173, the “E” residue at position 182, the “S” residue at position 242, the “G” residue at position 293, and the “DV” sequence beginning at position 295, (2) for “SP|O03067|ATPB_DICAN,” the “S” residue at position 180, the “S” residue at position 232, the “P” residue at position 235, the “S” residue at position 270, and the “G” residue at position 284, (3) for “SP|P06541|ATPB_CHLRE,” the “A” residue at position 240, the “A” residue at position 273, and the “A” residue at position 293, (4) for “SP|O47037|ATPB_PICAB,” the “A” residue at position 301, and (5) for “SP|Q5SCV8|ATPB_HUPLU,” the “G” residue at position 301.

In FIGS. 9A-9B, alignment by Clustal Omega (available at the uniprot.org website), “*” indicates 100% conserved identity. The first sequence from Arabidopsis is the reference sequence for the methods in Examples 4 through 7. The remaining sequences are approximately in order of evolutionary distance from Arabidopsis.

These and other objectives and features of the invention are apparent in the disclosure, which includes the above and ongoing written specification.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated.

The invention is not limited to the particular embodiments illustrated in the drawings and described above in detail. Those skilled in the art will recognize that other arrangements could be devised. The invention encompasses every possible combination of the various features of each embodiment disclosed. One or more of the elements described herein with respect to various embodiments can be implemented in a more separated or integrated manner than explicitly described, or even removed or rendered as inoperable in certain cases, as is useful in accordance with a particular application. While the invention has been described with reference to specific illustrative embodiments, modifications and variations of the invention may be constructed without departing from the spirit and scope of the invention as set forth in the following claims.

I/we claim:

1. A method for quantitative protein analysis of two or more plant species, the method comprising:
determining a set of common peptides that are common for the two or more plant species;
creating a set of isotope labeled peptides out of the set of common peptides;
adding a predefined amount of one or more labeled peptides from the set of isotope labeled peptides to a sample from one of the two or more plant species;
performing mass spectrometry to create first intensity values for a group of peptides from the sample and second intensity values for the one or more labeled peptides; and
calculating a quantitative amount of the group of peptides based on the first intensity values and the second intensity values.

2. The method of claim 1, wherein determining the common peptides is based on taxonomy comprising the two or more plant species.

3. The method of claim 2, wherein the taxonomy represents evolutionary relationships.

4. The method of claim 1, wherein determining the set of common peptides comprises:
determining, using at least one computer, digital data indicative of multiple species-specific sets of peptides based on digital sequence data from each of species in the two or more plant species, and
determining peptides that are common for the multiple sets of species-specific peptides,
wherein the at least one computer comprises at least one processor, and wherein the at least one processor is operatively connected to at least one non-transitory, computer readable medium having computer-executable instructions stored thereon.

5. The method of claim 1, wherein:
determining the set of common peptides is based on mass spectrometry data, the mass spectrometry data being indicative of multiple species-specific sets of peptides; and
the method further comprises determining peptides that are common for the multiple sets of species-specific peptides.

6. The method of claim 4, wherein the multiple sets of species-specific peptides comprise species-specific sets determined based on the digital sequence data.

7. The method of claim 5, wherein the multiple sets of species-specific peptides comprise species-specific sets determined based on the mass spectrometry data.

8. The method of claim 1, wherein the method is used for quantifying a protein complex.

9. The method of claim 8, wherein the protein complex is the same complex in the two or more species.

10. The method of claim 1, wherein the adding the predefined amount of the one or more labeled peptides further comprises adding the predefined amount of the one or more labeled peptides to a sample from a species in a group for which the set of common peptides was determined.

11. A kit for quantitative protein analysis of two or more plant species, the kit comprising:
two or more labeled peptides corresponding to peptides that are common between two or more plant species.

12. The kit of claim 11, wherein the peptides common to the two or more plant species are selected from a set of common peptides.

13. The kit of claim 11, wherein the peptides common to the two or more plant species are selected using a computational approach, a hybrid approach, and/or an empirical approach.

14. The kit of claim 11, wherein the two or more labeled peptides are selected from the group consisting of: SEQ ID NO. 54 through SEQ ID NO. 153, and combinations thereof.

15. The kit of claim 11, wherein the two or more plant species are two or more species of Rosids, and wherein the two or more labeled peptides are selected from the group consisting of: SEQ ID NO. 54 through SEQ ID NO. 453, and combinations thereof.

16. The kit of claim 11, further comprising two or more groups of labeled peptides corresponding to the peptides that are common between the two or more species, wherein the two or more groups are in a hierarchical relationship in relation to a taxonomy of species.

17. A method for quantitative protein analysis, the method comprising:
receiving, by at least one processor, mass spectrometry data comprising measurements with intensity values and corresponding mass-to-charge values;
based on the mass-to-charge values, identifying, by the at least one processor:
a first set of measurements that relate to labeled peptides from a set of common peptides that are common for two or more plant species; and
a second set of measurements that relate to sample peptides from the set of common peptides; and
calculating, by the at least one processor, a quantitative amount of the sample peptides based on the intensity values of the first set of measurements and the intensity values of the second set of measurements.

18. The method of claim 17, further comprising determining, by the at least one processor, the set of common peptides that are common for the two or more plant species.


SEQUENCE LISTING (465 sequences)

SEQ ID NO: 1 - protein, 13 aa, Artificial Sequence
  Conserved peptides across bacterial species
DVSGEGVQQALLK

SEQ ID NO: 2 - protein, 13 aa, Artificial Sequence
  Conserved peptides across bacterial species
NNPVLIGEPGVGK

SEQ ID NO: 3 - protein, 16 aa, Artificial Sequence
  Conserved peptides across bacterial species
RPIGSFIFLGPTGVGK

SEQ ID NO: 4 - protein, 11 aa, Artificial Sequence
  Conserved peptides across bacterial species
IIVDTYGGYAR

SEQ ID NO: 5 - protein, 12 aa, Artificial Sequence
  Conserved peptides across bacterial species
NFSIIAHIDHGK

SEQ ID NO: 6 - protein, 12 aa, Artificial Sequence
  Conserved peptides across bacterial species
VGIGPGSICTTR

SEQ ID NO: 7 - protein, 8 aa, Artificial Sequence
  Conserved peptides across bacterial species
AHILEGLR

SEQ ID NO: 8 - protein, 10 aa, Artificial Sequence
  Conserved peptides across bacterial species
EFTELGSGFK

SEQ ID NO: 9 - protein, 11 aa, Artificial Sequence
  Conserved peptides across bacterial species
SVGELLQNQFR

SEQ ID NO: 10 - protein, 11 aa, Artificial Sequence
  Conserved peptides across bacterial species
LSALGPGGLTR

SEQ ID NO: 11 - protein, 9 aa, Artificial Sequence
  Conserved peptides across bacterial species
LLHAIFGEK

SEQ ID NO: 12 - protein, 16 aa, Artificial Sequence
  Conserved peptides across bacterial species
STGPYSLVTQQPLGGK

SEQ ID NO: 13 - protein, 7 aa, Artificial Sequence
  Conserved peptides across bacterial species
AQFGGQR

SEQ ID NO: 14 - protein, 8 aa, Artificial Sequence
  Conserved peptides across bacterial species
KPETINYR

SEQ ID NO: 15 - protein, 11 aa, Artificial Sequence
  Conserved peptides across bacterial species
FATSDLNDLYR

SEQ ID NO: 16 - protein, 13 aa, Artificial Sequence
  Conserved peptides across bacterial species
GRPVTGPGNRPLK

SEQ ID NO: 17 - protein, 7 aa, Artificial Sequence
  Conserved peptides across bacterial species
SLSHMLK

SEQ ID NO: 18 - protein, 7 aa, Artificial Sequence
  Conserved peptides across bacterial species
IFGPVAR

SEQ ID NO: 19 - protein, 7 aa, Artificial Sequence
  Conserved peptides across bacterial species
GLMPNPK

SEQ ID NO: 20 - protein, 7 aa, Artificial Sequence
  Conserved peptides across bacterial species
ELIIGDR

SEQ ID NO: 21 - protein, 7 aa, Artificial Sequence
  Conserved peptides across bacterial species
DYLVPSR

SEQ ID NO: 22 - protein, 7 aa, Artificial Sequence
  Conserved peptides across bacterial species
KPNSALR

SEQ ID NO: 23 - protein, 7 aa, Artificial Sequence
  Conserved peptides across bacterial species
LVVSIAK

SEQ ID NO: 24 - protein, 10 aa, Artificial Sequence
  Conserved peptides across bacterial species
FSTYATWWIR

SEQ ID NO: 25 - protein, 7 aa, Artificial Sequence
  Conserved peptides across bacterial species
AIADQAR

SEQ ID NO: 26 - protein, 11 aa, Artificial Sequence
  Conserved peptides across bacterial species
IPVHMVETINK

SEQ ID NO: 27 - protein, 7 aa, Artificial Sequence
  Conserved peptides across bacterial species
FGLDDGR

SEQ ID NO: 28 - protein, 12 aa, Artificial Sequence
  Conserved peptides across bacterial species
ELPMEYAVEMNR

SEQ ID NO: 29 - protein, 15 aa, Artificial Sequence
  Conserved peptides across bacterial species
HYAHVDCPGHADYVK

SEQ ID NO: 30 - protein, 7 aa, Artificial Sequence
  Conserved peptides across bacterial species
GTVATGR

SEQ ID NO: 31 - protein, 7 aa, Artificial Sequence
  Conserved peptides across bacterial species
APGFGDR

SEQ ID NO: 32 - protein, 9 aa, Artificial Sequence
  Conserved peptides across bacterial species
IEDALNSTR

SEQ ID NO: 33 - protein, 7 aa, Artificial Sequence
  Conserved peptides across bacterial species
GGGGYIR

SEQ ID NO: 34 - protein, 8 aa, Artificial Sequence
  Conserved peptides across bacterial species
TMDIGGDK

SEQ ID NO: 35 - protein, 8 aa, Artificial Sequence
  Conserved peptides across bacterial species
NTTIPTSK

SEQ ID NO: 36 - protein, 9 aa, Artificial Sequence
  Conserved peptides across bacterial species
STLFNAITK

SEQ ID NO: 37 - protein, 10 aa, Artificial Sequence
  Conserved peptides across bacterial species
LLQGDVGSGK

SEQ ID NO: 38 - protein, 7 aa, Artificial Sequence
  Conserved peptides across bacterial species
GLLMGAR

SEQ ID NO: 39 - protein, 8 aa, Artificial Sequence
  Conserved peptides across bacterial species
DGLKPVQR

SEQ ID NO: 40 - protein, 8 aa, Artificial Sequence
  Conserved peptides across bacterial species
DGLKPVHR

SEQ ID NO: 41 - protein, 7 aa, Artificial Sequence
  Conserved peptides across bacterial species
GGTDGSK

SEQ ID NO: 42 - protein, 8 aa, Artificial Sequence
  Conserved peptides across bacterial species
VADNSGAR

SEQ ID NO: 43 - protein, 11 aa, Artificial Sequence
  Conserved peptides across bacterial species
GYGTTLGNSLR

SEQ ID NO: 44 - protein, 7 aa, Artificial Sequence
  Conserved peptides across bacterial species
LRPGEPK

SEQ ID NO: 45 - protein, 9 aa, Artificial Sequence
  Conserved peptides across bacterial species
ALMGANMQR

SEQ ID NO: 46 - protein, 7 aa, Artificial Sequence
  Conserved peptides across bacterial species
STPEGAR

SEQ ID NO: 47 - protein, 7 aa, Artificial Sequence
  Conserved peptides across bacterial species
EVIAFPK

SEQ ID NO: 48 - protein, 8 aa, Artificial Sequence
  Conserved peptides across bacterial species
GMTDTALK

SEQ ID NO: 49 - protein, 8 aa, Artificial Sequence
  Conserved peptides across bacterial species
VLTDAAIR

SEQ ID NO: 50 - protein, 7 aa, Artificial Sequence
  Conserved peptides across bacterial species
ENVIIGK

SEQ ID NO: 51 - protein, 10 aa, Artificial Sequence
  Conserved peptides across bacterial species
VEFFGDEIDR

SEQ ID NO: 52 - protein, 7 aa, Artificial Sequence
  Conserved peptides across bacterial species
GDWVISR

SEQ ID NO: 53 - protein, 14 aa, Artificial Sequence
  Conserved peptides across bacterial species
SSLAFDTLYAEGQR

SEQ ID NO: 54 - protein, 7 aa, Artificial Sequence
  Conserved peptides in yeast
LTGMAFR

SEQ ID NO: 55 - protein, 11 aa, Artificial Sequence
  Conserved peptides in yeast
IGLFGGAGVGK

SEQ ID NO: 56 - protein, 11 aa, Artificial Sequence
  Conserved peptides in yeast
LQIWDTAGQER

SEQ ID NO: 57 - protein, 8 aa, Artificial Sequence
  Conserved peptides in yeast
TITSSYYR

SEQ ID NO: 58 - protein, 7 aa, Artificial Sequence
  Conserved peptides in yeast
EIQTAVR

SEQ ID NO: 59 - protein, 12 aa, Artificial Sequence
  Conserved peptides in yeast
DNIQGITKPAIR

SEQ ID NO: 60 - protein, 7 aa, Artificial Sequence
  Conserved peptides in yeast
TLYGFGG

SEQ ID NO: 61 - protein, 12 aa, Artificial Sequence
  Conserved peptides in yeast
ELISNASDALDK

SEQ ID NO: 62 - protein, 10 aa, Artificial Sequence
  Conserved peptides in yeast
STTTGHLIYK

SEQ ID NO: 63 - protein, 8 aa, Artificial Sequence
  Conserved peptides in yeast
LPLQDVYK

SEQ ID NO: 64 - protein, 11 aa, Artificial Sequence
  Conserved peptides in yeast
IGGIGTVPVGR

SEQ ID NO: 65 - protein, 9 aa, Artificial Sequence
  Conserved peptides in yeast
QTVAVGVIK

SEQ ID NO: 66 - protein, 9 aa, Artificial Sequence
  Conserved peptides in yeast
EGLIDTAVK

SEQ ID NO: 67 - protein, 9 aa, Artificial Sequence
  Conserved peptides in yeast
EGLVDTAVK

SEQ ID NO: 68 - protein, 9 aa, Artificial Sequence
  Conserved peptides in yeast
EGIPPDQQR

SEQ ID NO: 69 - protein, 9 aa, Artificial Sequence
  Conserved peptides in yeast
ESTLHLVLR

SEQ ID NO: 70 - protein, 8 aa, Artificial Sequence
  Conserved peptides in yeast
VADFGLAR

SEQ ID NO: 71 - protein, 11 aa, Artificial Sequence
  Conserved peptides in yeast
MLDMGFEPQIR

SEQ ID NO: 72 - protein, 7 aa, Artificial Sequence
  Conserved peptides in yeast
SSALASK

SEQ ID NO: 73 - protein, 9 aa, Artificial Sequence
  Conserved peptides in yeast
YDLTVPFAR

SEQ ID NO: 74 - protein, 8 aa, Artificial Sequence
  Conserved peptides in yeast
TITTAYYR

SEQ ID NO: 75 - protein, 7 aa, Artificial Sequence
  Conserved peptides in yeast
QLWWGHR

SEQ ID NO: 76 - protein, 9 aa, Artificial Sequence
  Conserved peptides in yeast
AGVSQVLNR

SEQ ID NO: 77 - protein, 9 aa, Artificial Sequence
  Conserved peptides in yeast
NTYQSAMGK

SEQ ID NO: 78 - protein, 11 aa, Artificial Sequence
  Conserved peptides in yeast
LLLLGAGESGK

SEQ ID NO: 79 - protein, 11 aa, Artificial Sequence
  Conserved peptides in yeast
VEIIANDQGNR

SEQ ID NO: 80 - protein, 13 aa, Artificial Sequence
  Conserved peptides in yeast
TTPSYVAFTDTER

SEQ ID NO: 81 - protein, 16 aa, Artificial Sequence
  Conserved peptides in yeast
IINEPTAAAIAYGLDK

SEQ ID NO: 82 - protein, 7 aa, Artificial Sequence
  Conserved peptides in yeast
ITITNDK

SEQ ID NO: 83 - protein, 7 aa, Artificial Sequence
  Conserved peptides in yeast
FDLMYAK

SEQ ID NO: 84 - protein, 8 aa, Artificial Sequence
  Conserved peptides in yeast
GGMQIFVK

SEQ ID NO: 85 - protein, 7 aa, Artificial Sequence
  Conserved peptides in yeast
NTTIPTK

SEQ ID NO: 86 - protein, 7 aa, Artificial Sequence
  Conserved peptides in yeast
VHGSLAR

SEQ ID NO: 87 - protein, 8 aa, Artificial Sequence
  Conserved peptides in yeast
ECADLWPR

SEQ ID NO: 88 - protein, 9 aa, Artificial Sequence
  Conserved peptides in yeast
DELTLEGIK

SEQ ID NO: 89 - protein, 7 aa, Artificial Sequence
  Conserved peptides in yeast
IDHYLGK

SEQ ID NO: 90 - protein, 7 aa, Artificial Sequence
  Conserved peptides in yeast
NAEYNPK

SEQ ID NO: 91 - protein, 7 aa, Artificial Sequence
  Conserved peptides in yeast
ALCTGEK

SEQ ID NO: 92 - protein, 7 aa, Artificial Sequence
  Conserved peptides in yeast
DVIAFPK

SEQ ID NO: 93 - protein, 9 aa, Artificial Sequence
  Conserved peptides in yeast
SAIGEGMTR

SEQ ID NO: 94 - protein, 7 aa, Artificial Sequence
  Conserved peptides in yeast
DNNLLGK

SEQ ID NO: 95 - protein, 12 aa, Artificial Sequence
  Conserved peptides in yeast
YFPTQALNFAFK

SEQ ID NO: 96 - protein, 8 aa, Artificial Sequence
  Conserved peptides in yeast
APGFGDNR

SEQ ID NO: 97 - protein, 8 aa, Artificial Sequence
  Conserved peptides in yeast
AGAFDQLK

SEQ ID NO: 98 - protein, 7 aa, Artificial Sequence
  Conserved peptides in yeast
GYIDLSK

SEQ ID NO: 99 - protein, 8 aa, Artificial Sequence
  Conserved peptides in yeast
TTLLHMLK

SEQ ID NO: 100 - protein, 10 aa, Artificial Sequence
  Conserved peptides in yeast
HITIFSPEGR

SEQ ID NO: 101 - protein, 9 aa, Artificial Sequence
  Conserved peptides in yeast
NTYQCAMGK

SEQ ID NO: 102 - protein, 14 aa, Artificial Sequence
  Conserved peptides in yeast
QITQVYGFYDECLR

SEQ ID NO: 103 - protein, 12 aa, Artificial Sequence
  Conserved peptides in yeast
NIGISAHIDSGK

SEQ ID NO: 104 - protein, 9 aa, Artificial Sequence
  Conserved peptides in yeast
GSLPWQGLK

SEQ ID NO: 105 - protein, 16 aa, Artificial Sequence
  Conserved peptides in yeast
VAIHEAMEQQTISIAK

SEQ ID NO: 106 - protein, 12 aa, Artificial Sequence
  Conserved peptides in yeast
NMSVIAHVDHGK

SEQ ID NO: 107 - protein, 16 aa, Artificial Sequence
  Conserved peptides in yeast
QATINIGTIGHVAHGK

SEQ ID NO: 108 - protein, 7 aa, Artificial Sequence
  Conserved peptides in yeast
LGYANAK

SEQ ID NO: 109 - protein, 13 aa, Artificial Sequence
  Conserved peptides in yeast
QSLETICLLLAYK

SEQ ID NO: 110 - protein, 10 aa, Artificial Sequence
  Conserved peptides in yeast
GNHECASINR

SEQ ID NO: 111 - protein, 9 aa, Artificial Sequence
  Conserved peptides in yeast
IYGFYDECK

SEQ ID NO: 112 - protein, 8 aa, Artificial Sequence
  Conserved peptides in yeast
HLTGEFEK

SEQ ID NO: 113 - protein, 13 aa, Artificial Sequence
  Conserved peptides in yeast
VCENIPIVLCGNK

SEQ ID NO: 114 - protein, 10 aa, Artificial Sequence
  Conserved peptides in yeast
FQSLGVAFYR

SEQ ID NO: 115 - protein, 7 aa, Artificial Sequence
  Conserved peptides in yeast
YLGEGPR

SEQ ID NO: 116 - protein, 7 aa, Artificial Sequence
  Conserved peptides in yeast
VIMATNR

SEQ ID NO: 117 - protein, 9 aa, Artificial Sequence
  Conserved peptides in yeast
VIGSELVQK

SEQ ID NO: 118 - protein, 7 aa, Artificial Sequence
  Conserved peptides in yeast
YVGEGAR

SEQ ID NO: 119 - protein, 10 aa, Artificial Sequence
  Conserved peptides in yeast
TGHSGTLDPK

SEQ ID NO: 120 - protein, 11 aa, Artificial Sequence
  Conserved peptides in yeast
FTLWWSPTINR

SEQ ID NO: 121 - protein, 8 aa, Artificial Sequence
  Conserved peptides in yeast
ISLIQIFR

SEQ ID NO: 122 - protein, 11 aa, Artificial Sequence
  Conserved peptides in yeast
IIHTSVWAGQK

SEQ ID NO: 123 - protein, 7 aa, Artificial Sequence
  Conserved peptides in yeast
LAEQAER

SEQ ID NO: 124 - protein, 8 aa, Artificial Sequence
  Conserved peptides in yeast
NLLSVAYK

SEQ ID NO: 125 - protein, 10 aa, Artificial Sequence
  Conserved peptides in yeast
DSTLIMQLLR

SEQ ID NO: 126 - protein, 10 aa, Artificial Sequence
  Conserved peptides in yeast
DIVFAASLYL

SEQ ID NO: 127 - protein, 11 aa, Artificial Sequence
  Conserved peptides in yeast
AQIWDTAGQER

SEQ ID NO: 128 - protein, 8 aa, Artificial Sequence
  Conserved peptides in yeast
AITSAYYR

SEQ ID NO: 129 - protein, 8 aa, Artificial Sequence
  Conserved peptides in yeast
LCDFGSAK

SEQ ID NO: 130 - protein, 8 aa, Artificial Sequence
  Conserved peptides in yeast
IADFGLAK

SEQ ID NO: 131 - protein, 7 aa, Artificial Sequence
  Conserved peptides in yeast
GANEATK

SEQ ID NO: 132 - protein, 7 aa, Artificial Sequence
  Conserved peptides in yeast
LIGDAAK

SEQ ID NO: 133 - protein, 7 aa, Artificial Sequence
  Conserved peptides in yeast
DTQCGFK

SEQ ID NO: 134 - protein, 9 aa, Artificial Sequence
  Conserved peptides in yeast
MLSCAGADR

SEQ ID NO: 135 - protein, 8 aa, Artificial Sequence
  Conserved peptides in yeast
ICDFGLAR

SEQ ID NO: 136 - protein, 13 aa, Artificial Sequence
  Conserved peptides in yeast
AVAVVVDPIQSVK

SEQ ID NO: 137 - protein, 7 aa, Artificial Sequence
  Conserved peptides in yeast
VVIDAFR

SEQ ID NO: 138 - protein, 9 aa, Artificial Sequence
  Conserved peptides in yeast
YMTDGMLLR

SEQ ID NO: 139 - protein, 12 aa, Artificial Sequence
  Conserved peptides in yeast
GVLLYGPPGTGK

SEQ ID NO: 140 - protein, 7 aa, Artificial Sequence
  Conserved peptides in yeast
YIGESAR

SEQ ID NO: 141 - protein, 12 aa, Artificial Sequence
  Conserved peptides in yeast
LTSLGVIGALVK

SEQ ID NO: 142 - protein, 7 aa, Artificial Sequence
  Conserved peptides in yeast
GAFGEVR

SEQ ID NO: 143 - protein, 10 aa, Artificial Sequence
  Conserved peptides in yeast
CATITPDEAR

SEQ ID NO: 144 - protein, 7 aa, Artificial Sequence
  Conserved peptides in yeast
SPNGTIR

SEQ ID NO: 145 - protein, 10 aa, Artificial Sequence
  Conserved peptides in yeast
AGFAGDDAPR

SEQ ID NO: 146 - protein, 11 aa, Artificial Sequence
  Conserved peptides in yeast
IWHHTFYNELR

SEQ ID NO: 147 - protein, 7 aa, Artificial Sequence
  Conserved peptides in yeast
STELLIR

SEQ ID NO: 148 - protein, 7 aa, Artificial Sequence
  Conserved peptides in yeast
EIAQDFK

SEQ ID NO: 149 - protein, 9 aa, Artificial Sequence
  Conserved peptides in yeast
LGLTATLVR

SEQ ID NO: 150 - protein, 7 aa, Artificial Sequence
  Conserved peptides in yeast
ELFVMAR

SEQ ID NO: 151 - protein, 9 aa, Artificial Sequence
  Conserved peptides in yeast
GTGLYELWK

SEQ ID NO: 152 - protein, 9 aa, Artificial Sequence
  Conserved peptides in yeast
TEALTQAFR

SEQ ID NO: 153 - protein, 9 aa, Artificial Sequence
  Conserved peptides in yeast
AGLQFPVGR

SEQ ID NO: 154 - protein, 12 aa, Artificial Sequence
  Conserved rosid peptides
NPFAPTLHFNYR

SEQ ID NO: 155 - protein, 10 aa, Artificial Sequence
  Conserved rosid peptides
LNLFPGYMER

SEQ ID NO: 156 - protein, 7 aa, Artificial Sequence
  Conserved rosid peptides
FAALPWR

SEQ ID NO: 157 - protein, 13 aa, Artificial Sequence
  Conserved rosid peptides
AAVIGDTIGDPLK

SEQ ID NO: 158 - protein, 11 aa, Artificial Sequence
  Conserved rosid peptides
AADVGADLVGK

SEQ ID NO: 159 - protein, 16 aa, Artificial Sequence
  Conserved rosid peptides
TDALDAAGNTTAAIGK

SEQ ID NO: 160 - protein, 12 aa, Artificial Sequence
  Conserved rosid peptides
INVYYNEASGGR

SEQ ID NO: 161 - protein, 10 aa, Artificial Sequence
  Conserved rosid peptides
VLILGGGPNR

SEQ ID NO: 162 - protein, 11 aa, Artificial Sequence
  Conserved rosid peptides
FYGEVTQQMLK

SEQ ID NO: 163 - protein, 14 aa, Artificial Sequence
  Conserved rosid peptides
VVAWYDNEWGYSQR

SEQ ID NO: 164 - protein, 13 aa, Artificial Sequence
  Conserved rosid peptides
TIEAEAAHGTVTR

SEQ ID NO: 165 - protein, 9 aa, Artificial Sequence
  Conserved rosid peptides
MDFPDPVIK

SEQ ID NO: 166 - protein, 13 aa, Artificial Sequence
  Conserved rosid peptides
VEANVGAPQVNYR

SEQ ID NO: 167 - protein, 12 aa, Artificial Sequence
  Conserved rosid peptides
LAQEDPSFHFSR

SEQ ID NO: 168 - protein, 18 aa, Artificial Sequence
  Conserved rosid peptides
INIIDTPGHVDFTLEVER

SEQ ID NO: 169 - protein, 20 aa, Artificial Sequence
  Conserved rosid peptides
IGEVHEGTATMDWMEQEQER

SEQ ID NO: 170 - protein, 8 aa, Artificial Sequence
  Conserved rosid peptides
AFGMELLR

SEQ ID NO: 171 - protein, 11 aa, Artificial Sequence
  Conserved rosid peptides
ITACLDPDGWK

SEQ ID NO: 172 - protein, 13 aa, Artificial Sequence
  Conserved rosid peptides
GPTPEPLCQVMLR

SEQ ID NO: 173 - protein, 12 aa, Artificial Sequence
  Conserved rosid peptides
LSGTGSEGATIR

SEQ ID NO: 174 - protein, 13 aa, Artificial Sequence
  Conserved rosid peptides
EDDLNEIVQLVGK

SEQ ID NO: 175 - protein, 13 aa, Artificial Sequence
  Conserved rosid peptides
HFPSVNWLISYSK

SEQ ID NO: 176 - protein, 24 aa, Artificial Sequence
  Conserved rosid peptides
VLDALFPSVLGGTCAIPGAFGCGK

SEQ ID NO: 177 - protein, 12 aa, Artificial Sequence
  Conserved rosid peptides
ELVSNASDALDK

SEQ ID NO: 178 - protein, 10 aa, Artificial Sequence
  Conserved rosid peptides
VVNDGVTIAR

SEQ ID NO: 179 - protein, 21 aa, Artificial Sequence
  Conserved rosid peptides
FQMEPNTGVTFDDVAGVDEAK

SEQ ID NO: 180 - protein, 11 aa, Artificial Sequence
  Conserved rosid peptides
VPLILGIWGGK

SEQ ID NO: 181 - protein, 15 aa, Artificial Sequence
  Conserved rosid peptides
MCCLFINDLDAGAGR

SEQ ID NO: 182 - protein, 23 aa, Artificial Sequence
  Conserved rosid peptides
MGINPIMMSAGELESGNAGEPAK

SEQ ID NO: 183 - protein, 12 aa, Artificial Sequence
  Conserved rosid peptides
DVAWAPNLGLPK

SEQ ID NO: 184 - protein, 20 aa, Artificial Sequence
  Conserved rosid peptides
IGLAGLAVMGQNLALNIAEK

SEQ ID NO: 185 - protein, 12 aa, Artificial Sequence
  Conserved rosid peptides
GVLLVGPPGTGK

SEQ ID NO: 186 - protein, 10 aa, Artificial Sequence
  Conserved rosid peptides
GSAITGPIGK

SEQ ID NO: 187 - protein, 8 aa, Artificial Sequence
  Conserved rosid peptides
NLYIISVK

SEQ ID NO: 188 - protein, 20 aa, Artificial Sequence
  Conserved rosid peptides
MSLGLPVAATVNCADNTGAK

SEQ ID NO: 189 - protein, 8 aa, Artificial Sequence
  Conserved rosid peptides
LLILTDPR

SEQ ID NO: 190 - protein, 10 aa, Artificial Sequence
  Conserved rosid peptides
ADILDPALMR

SEQ ID NO: 191 - protein, 11 aa, Artificial Sequence
  Conserved rosid peptides
VGSSEAALLAK

SEQ ID NO: 192 - protein, 9 aa, Artificial Sequence
  Conserved rosid peptides
QAVDISPLR

SEQ ID NO: 193 - protein, 15 aa, Artificial Sequence
  Conserved rosid peptides
TIAECLADELINAAK

SEQ ID NO: 194 - protein, 10 aa, Artificial Sequence
  Conserved rosid peptides
TMGPVPLPTK

SEQ ID NO: 195 - protein, 12 aa, Artificial Sequence
  Conserved rosid peptides
VIDGAIGAEWLK

SEQ ID NO: 196 - protein, 11 aa, Artificial Sequence
  Conserved rosid peptides
LFGVTTLDVVR

SEQ ID NO: 197 - protein, 12 aa, Artificial Sequence
  Conserved rosid peptides
DDLFNINAGIVK

SEQ ID NO: 198 - protein, 9 aa, Artificial Sequence
  Conserved rosid peptides
VVDIVDTFR

SEQ ID NO: 199 - protein, 7 aa, Artificial Sequence
  Conserved rosid peptides
LLDASHR

SEQ ID NO: 200 - protein, 8 aa, Artificial Sequence
  Conserved rosid peptides
VAINGFGR

SEQ ID NO: 201 - protein, 14 aa, Artificial Sequence
  Conserved rosid peptides
GTMTTTHSYTGDQR

SEQ ID NO: 202 - protein, 14 aa, Artificial Sequence
  Conserved rosid peptides
VIAWYDNEWGYSQR

SEQ ID NO: 203 - protein, 11 aa, Artificial Sequence
  Conserved rosid peptides
MSILSTAGSGK

SEQ ID NO: 204 - protein, 8 aa, Artificial Sequence
  Conserved rosid peptides
QIASLVQR

SEQ ID NO: 205 - protein, 12 aa, Artificial Sequence
  Conserved rosid peptides
TLLYGGIYGYPR

SEQ ID NO: 206 - protein, 26 aa, Artificial Sequence
  Conserved rosid peptides
GHSYSEIINESVIESVDSLNPFMHAR

SEQ ID NO: 207 - protein, 9 aa, Artificial Sequence
  Conserved rosid peptides
DCEEWFFDR

SEQ ID NO: 208 - protein, 14 aa, Artificial Sequence
  Conserved rosid peptides
NVTILDQSPHQLAK

SEQ ID NO: 209 - protein, 9 aa, Artificial Sequence
  Conserved rosid peptides
VENYFFDIR

SEQ ID NO: 210 - protein, 11 aa, Artificial Sequence
  Conserved rosid peptides
ILFLGLDNAGK

SEQ ID NO: 211 - protein, 9 aa, Artificial Sequence
  Conserved rosid peptides
EQCLALGTR

SEQ ID NO: 212 - protein, 15 aa, Artificial Sequence
  Conserved rosid peptides
EQIFEMPTGGAAIMR

SEQ ID NO: 213 - protein, 7 aa, Artificial Sequence
  Conserved rosid peptides
VELLYTK

SEQ ID NO: 214 - protein, 19 aa, Artificial Sequence
  Conserved rosid peptides
QAFDEAIAELDTLGEESYK

SEQ ID NO: 215 - protein, 10 aa, Artificial Sequence
  Conserved rosid peptides
GDEELDTLIK

SEQ ID NO: 216 - protein, 9 aa, Artificial Sequence
  Conserved rosid peptides
HSLPDGLMR

SEQ ID NO: 217 - protein, 8 aa, Artificial Sequence
  Conserved rosid peptides
YTLDVDLK

SEQ ID NO: 218 - protein, 11 aa, Artificial Sequence
  Conserved rosid peptides
YIIIGDTGVGK

SEQ ID NO: 219 - protein, 7 aa, Artificial Sequence
  Conserved rosid peptides
MVMPGDR

SEQ ID NO: 220 - protein, 11 aa, Artificial Sequence
  Conserved rosid peptides
YDEIDAAPEER

SEQ ID NO: 221 - protein, 16 aa, Artificial Sequence
  Conserved rosid peptides
GITINTATVEYETENR

SEQ ID NO: 222 - protein, 15 aa, Artificial Sequence
  Conserved rosid peptides
HSPFFAGYRPQFYMR

SEQ ID NO: 223 - protein, 9 aa, Artificial Sequence
  Conserved rosid peptides
FGWSANMER

SEQ ID NO: 224 - protein, 8 aa, Artificial Sequence
  Conserved rosid peptides
ILLESAIR

SEQ ID NO: 225 - protein, 9 aa, Artificial Sequence
  Conserved rosid peptides
EWTAWDIAR

SEQ ID NO: 226 - protein, 11 aa, Artificial Sequence
  Conserved rosid peptides
EETGAGMMDCK

SEQ ID NO: 227 - protein, 10 aa, Artificial Sequence
  Conserved rosid peptides
ELSEIAEQAK

SEQ ID NO: 228 - protein, 12 aa, Artificial Sequence
  Conserved rosid peptides
TIEVNNTDAEGR

SEQ ID NO: 229 - protein, 8 aa, Artificial Sequence
  Conserved rosid peptides
VDNVYGDR

SEQ ID NO: 230 - protein, 19 aa, Artificial Sequence
  Conserved rosid peptides
TFCIPHGGGGPGMGPIGVK

SEQ ID NO: 231 - protein, 12 aa, Artificial Sequence
  Conserved rosid peptides
SIATLAITTLLK

SEQ ID NO: 232 - protein, 11 aa, Artificial Sequence
  Conserved rosid peptides
LADGLFLESCR

SEQ ID NO: 233 - protein, 19 aa, Artificial Sequence
  Conserved rosid peptides
VLLQDFTGVPAVVDLACMR

SEQ ID NO: 234 - protein, 12 aa, Artificial Sequence
  Conserved rosid peptides
TSLAPGSGVVTK

SEQ ID NO: 235 - protein, 15 aa, Artificial Sequence
  Conserved rosid peptides
IALTTAEYLAYECGK

SEQ ID NO: 236 - protein, 20 aa, Artificial Sequence
  Conserved rosid peptides
IPLFSAAGLPHNEIAAQICR

SEQ ID NO: 237 - protein, 8 aa, Artificial Sequence
  Conserved rosid peptides
ALQNTCLK

SEQ ID NO: 238 - protein, 9 aa, Artificial Sequence
  Conserved rosid peptides
DFSTAILER

SEQ ID NO: 239 - protein, 12 aa, Artificial Sequence
  Conserved rosid peptides
GILLYGPPGSGK

SEQ ID NO: 240 - protein, 13 aa, Artificial Sequence
  Conserved rosid peptides
IVSQLLTLMDGLK

SEQ ID NO: 241 - protein, 8 aa, Artificial Sequence
  Conserved rosid peptides
WPLAQPMR

SEQ ID NO: 242 - protein, 13 aa, Artificial Sequence
  Conserved rosid peptides
FCTGGMSLGAISR

SEQ ID NO: 243 - protein, 9 aa, Artificial Sequence
  Conserved rosid peptides
EMIESGVIK

SEQ ID NO: 244 - protein, 13 aa, Artificial Sequence
  Conserved rosid peptides
TVLIMELINNVAK

SEQ ID NO: 245 - protein, 14 aa, Artificial Sequence
  Conserved rosid peptides
FTQANSEVSALLGR

SEQ ID NO: 246 - protein, 15 aa, Artificial Sequence
  Conserved rosid peptides
CALVYGQMNEPPGAR

SEQ ID NO: 247 - protein, 14 aa, Artificial Sequence
  Conserved rosid peptides
ANTFVAEVLGLDPR

SEQ ID NO: 248 - protein, 16 aa, Artificial Sequence
  Conserved rosid peptides
YPIEHGIVSNWDDMEK

SEQ ID NO: 249 - protein, 10 aa, Artificial Sequence
  Conserved rosid peptides
VGDIMTEENK

SEQ ID NO: 250 - protein, 9 aa, Artificial Sequence
  Conserved rosid peptides
LNLGVGAYR

SEQ ID NO: 251 - protein, 8 aa, Artificial Sequence
  Conserved rosid peptides
TAAAPIER

SEQ ID NO: 252 - protein, 10 aa, Artificial Sequence
  Conserved rosid peptides
MMMTSGEAVK

SEQ ID NO: 253 - protein, 10 aa, Artificial Sequence
  Conserved rosid peptides
DLQMVNLTLR

SEQ ID NO: 254 - protein, 11 aa, Artificial Sequence
  Conserved rosid peptides
ILMVGLDAAGK

SEQ ID NO: 255 - protein, 14 aa, Artificial Sequence
  Conserved rosid peptides
NISFTVWDVGGQDK

SEQ ID NO: 256 - protein, 9 aa, Artificial Sequence
  Conserved rosid peptides
IFEGEALLR

SEQ ID NO: 257 - protein, 11 aa, Artificial Sequence
  Conserved rosid peptides
DELDIVIPTIR

SEQ ID NO: 258 - protein, 10 aa, Artificial Sequence
  Conserved rosid peptides
AFSVFLFNSK

SEQ ID NO: 259 - protein, 11 aa, Artificial Sequence
  Conserved rosid peptides
NLYLSCDPYMR

SEQ ID NO: 260 - protein, 10 aa, Artificial Sequence
  Conserved rosid peptides
YLFAGVVDGR

SEQ ID NO: 261 - protein, 8 aa, Artificial Sequence
  Conserved rosid peptides
TLLVADPR

SEQ ID NO: 262 - protein, 15 aa, Artificial Sequence
  Conserved rosid peptides
AVFVDLEPTVIDEVR

SEQ ID NO: 263 - protein, 9 aa, Artificial Sequence
  Conserved rosid peptides
SWLAFAAQK

SEQ ID NO: 264 - protein, 16 aa, Artificial Sequence
  Conserved rosid peptides
YGAGIGPGVYDIHSPR

SEQ ID NO: 265 - protein, 16 aa, Artificial Sequence
  Conserved rosid peptides
GMLTGPVTILNWSFVR

SEQ ID NO: 266 - protein, 10 aa, Artificial Sequence
  Conserved rosid peptides
GFGILDVGYR

SEQ ID NO: 267 - protein, 10 aa, Artificial Sequence
  Conserved rosid peptides
LAVNLIPFPR

SEQ ID NO: 268 - protein, 14 aa, Artificial Sequence
  Conserved rosid peptides
LHFFMVGFAPLTSR

SEQ ID NO: 269 - protein, 18 aa, Artificial Sequence
  Conserved rosid peptides
GHYTEGAELIDSVLDVVR

SEQ ID NO: 270 - protein, 7 aa, Artificial Sequence
  Conserved rosid peptides
IWLVDSK

SEQ ID NO: 271 - protein, 18 aa, Artificial Sequence
  Conserved rosid peptides
ILGLGDLGCQGMGIPVGK

SEQ ID NO: 272 - protein, 7 aa, Artificial Sequence
  Conserved rosid peptides
GAMIFFR

SEQ ID NO: 273 - protein, 9 aa, Artificial Sequence
  Conserved rosid peptides
MGTPALTSR

SEQ ID NO: 274 - protein, 11 aa, Artificial Sequence
  Conserved rosid peptides
LIVAGASAYAR

SEQ ID NO: 275 - protein, 16 aa, Artificial Sequence
  Conserved rosid peptides
NTVPGDVSAMVPGGIR

SEQ ID NO: 276 - protein, 14 aa, Artificial Sequence
  Conserved rosid peptides
ISAVSIFFETMPYR

SEQ ID NO: 277 - protein, 9 aa, Artificial Sequence
  Conserved rosid peptides
AEEMAQTFR

SEQ ID NO: 278 - protein, 12 aa, Artificial Sequence
  Conserved rosid peptides
GLCAIAQAESLR

SEQ ID NO: 279 - protein, 12 aa, Artificial Sequence
  Conserved rosid peptides
ENPGCLFIATNR

SEQ ID NO: 280 - protein, 27 aa, Artificial Sequence
  Conserved rosid peptides
WNYDGSSTGQAPGEDSEVILYPQAIFK

SEQ ID NO: 281 - protein, 10 aa, Artificial Sequence
  Conserved rosid peptides
YEEMVEFMEK

SEQ ID NO: 282 - protein, 9 aa, Artificial Sequence
  Conserved rosid peptides
GFPISVYNR

SEQ ID NO: 283 - protein, 8 aa, Artificial Sequence
  Conserved rosid peptides
LESGLYSR

SEQ ID NO: 284 - protein, 9 aa, Artificial Sequence
  Conserved rosid peptides
DEISDALER

SEQ ID NO: 285 - protein, 11 aa, Artificial Sequence
  Conserved rosid peptides
LELQEVVDFLK

SEQ ID NO: 286 - protein, 22 aa, Artificial Sequence
  Conserved rosid peptides
TPGFTGADLQNLMNEAAILAAR

SEQ ID NO: 287 - protein, 8 aa, Artificial Sequence
  Conserved rosid peptides
YEGVILNK

SEQ ID NO: 288 - protein, 10 aa, Artificial Sequence
  Conserved rosid peptides
AMQLLESGLK

SEQ ID NO: 289 - protein, 10 aa, Artificial Sequence
  Conserved rosid peptides
IGGVMIMGDR

SEQ ID NO: 290 - protein, 14 aa, Artificial Sequence
  Conserved rosid peptides
INMVDLPLGATEDR

SEQ ID NO: 291 - protein, 21 aa, Artificial Sequence
  Conserved rosid peptides
FILIGSGNPEEGELRPQLLDR

SEQ ID NO: 292 - protein, 18 aa, Artificial Sequence
  Conserved rosid peptides
MLDADVTDSVIGEGCVIK

SEQ ID NO: 293 - protein, 8 aa, Artificial Sequence
  Conserved rosid peptides
IAGLEVLR

SEQ ID NO: 294 - protein, 11 aa, Artificial Sequence
  Conserved rosid peptides
FEELCSDLLDR

SEQ ID NO: 295 - protein, 13 aa, Artificial Sequence
  Conserved rosid peptides
QFAAEEISAQVLR

SEQ ID NO: 296 - protein, 8 aa, Artificial Sequence
  Conserved rosid peptides
LDEMIVFR

SEQ ID NO: 297 - protein, 9 aa, Artificial Sequence
  Conserved rosid peptides
LDMSEFMER

SEQ ID NO: 298 - protein, 10 aa, Artificial Sequence
  Conserved rosid peptides
VIMLAQEEAR

SEQ ID NO: 299 - protein, 10 aa, Artificial Sequence
  Conserved rosid peptides
IGFDLDYDEK

SEQ ID NO: 300 - protein, 14 aa, Artificial Sequence
  Conserved rosid peptides
VITLDMGLLVAGTK

SEQ ID NO: 301 - protein, 15 aa, Artificial Sequence
  Conserved rosid peptides
ALAAYYFGSEEAMIR

SEQ ID NO: 302 - protein, 17 aa, Artificial Sequence
  Conserved rosid peptides
NTLLIMTSNVGSSVIEK

SEQ ID NO: 303 - protein, 17 aa, Artificial Sequence
  Conserved rosid peptides
AHPDVFNMMLQILEDGR

SEQ ID NO: 304 - protein, 22 aa, Artificial Sequence
  Conserved rosid peptides
LIGSPPGYVGYTEGGQLTEAVR

SEQ ID NO: 305 - protein, 8 aa, Artificial Sequence
  Conserved rosid peptides
GLVVPVIR

SEQ ID NO: 306 - protein, 8 aa, Artificial Sequence
  Conserved rosid peptides
EEYAAFYK

SEQ ID NO: 307 - protein, 10 aa, Artificial Sequence
  Conserved rosid peptides
AVENSPFLEK

SEQ ID NO: 308 - protein, 12 aa, Artificial Sequence
  Conserved rosid peptides
ADLVNNLGTIAR

SEQ ID NO: 309 - protein, 10 aa, Artificial Sequence
  Conserved rosid peptides
EDQLEYLEER

SEQ ID NO: 310 - protein, 14 aa, Artificial Sequence
  Conserved rosid peptides
GIVDSEDLPLNISR

SEQ ID NO: 311 - protein, 9 aa, Artificial Sequence
  Conserved rosid peptides
VEDALNATK

SEQ ID NO: 312 - protein, 13 aa, Artificial Sequence
  Conserved rosid peptides
VVAAGANPVLITR

SEQ ID NO: 313 - protein, 14 aa, Artificial Sequence
  Conserved rosid peptides
EVELEDPVENIGAK

SEQ ID NO: 314 - protein, 17 aa, Artificial Sequence
  Conserved rosid peptides
AAVEEGIVVGGGCTLLR

SEQ ID NO: 315 - protein, 19 aa, Artificial Sequence
  Conserved rosid peptides
LSGGVAVIQVGAQTETELK

SEQ ID NO: 316 - protein, 10 aa, Artificial Sequence
  Conserved rosid peptides
LGDIIPADAR

SEQ ID NO: 317 - protein, 12 aa, Artificial Sequence
  Conserved rosid peptides
ADGFAGVFPEHK

SEQ ID NO: 318 - protein, 15 aa, Artificial Sequence
  Conserved rosid peptides
ADIGIAVADATDAAR

SEQ ID NO: 319 - protein, 17 aa, Artificial Sequence
  Conserved rosid peptides
MTAIEEMAGMDVLCSDK

SEQ ID NO: 320 - protein, 10 aa, Artificial Sequence
  Conserved rosid peptides
GYSFTTTAER

SEQ ID NO: 321 - protein, 11 aa, Artificial Sequence
  Conserved rosid peptides
HTGVMVGMGQK

SEQ ID NO: 322 - protein, 18 aa, Artificial Sequence
  Conserved rosid peptides
VAPEEHPVLLTEAPLNPK

SEQ ID NO: 323 - protein, 11 aa, Artificial Sequence
  Conserved rosid peptides
LLLIGDSGVGK

SEQ ID NO: 324 - protein, 8 aa, Artificial Sequence
  Conserved rosid peptides
IVVELNGR

SEQ ID NO: 325 - protein, 9 aa, Artificial Sequence
  Conserved rosid peptides
LVLPGELAK

SEQ ID NO: 326 - protein, 15 aa, Artificial Sequence
  Conserved rosid peptides
AMGIMNSFINDIFEK

SEQ ID NO: 327 - protein, 10 aa, Artificial Sequence
  Conserved rosid peptides
DAVTYTEHAR

SEQ ID NO: 328 - protein, 10 aa, Artificial Sequence
  Conserved rosid peptides
ISGLIYEETR

SEQ ID NO: 329 - protein, 12 aa, Artificial Sequence
  Conserved rosid peptides
TVTAMDVVYALK

SEQ ID NO: 330 - protein, 8 aa, Artificial Sequence
  Conserved rosid peptides
STNLDWYK

SEQ ID NO: 331 - protein, 12 aa, Artificial Sequence
  Conserved rosid peptides
EHALLAFTLGVK

SEQ ID NO: 332 - protein, 12 aa, Artificial Sequence
  Conserved rosid peptides
YYCTVIDAPGHR

SEQ ID NO: 333 - protein, 29 aa, Artificial Sequence
  Conserved rosid peptides
NMITGTSQADCAVLIIDSTTGGFEAGISK

SEQ ID NO: 334 - protein, 19 aa, Artificial Sequence
  Conserved rosid peptides
VIEAGANALVAGSAVFGAK

SEQ ID NO: 335 - protein, 8 aa, Artificial Sequence
  Conserved rosid peptides
CGSNVFWK

SEQ ID NO: 336 - protein, 13 aa, Artificial Sequence
  Conserved rosid peptides
FPENFTGCQDLAK

SEQ ID NO: 337 - protein, 11 aa, Artificial Sequence
  Conserved rosid peptides
ALLEVVESGGK

SEQ ID NO: 338 - protein, 7 aa, Artificial Sequence
  Conserved rosid peptides
LDFAVSR

SEQ ID NO: 339 - protein, 12 aa, Artificial Sequence
  Conserved peptides
LIFQYASFNNSR

SEQ ID NO: 340 - protein, 11 aa, Artificial Sequence
  Conserved peptides
VINTWADIINR

SEQ ID NO: 341 - protein, 10 aa, Artificial Sequence
  Conserved peptides
AYDFVSQEIR

SEQ ID NO: 342 - protein, 9 aa, Artificial Sequence
  Conserved peptides
NILLNEGIR

SEQ ID NO: 343 - protein, 13 aa, Artificial Sequence
  Conserved peptides
LAFYDYIGNNPAK

SEQ ID NO: 344 - protein, 11 aa, Artificial Sequence
  Conserved peptides
VHTVVLNDPGR

SEQ ID NO: 345 - protein, 8 aa, Artificial Sequence
  Conserved peptides
APWLEPLR

SEQ ID NO: 346 - protein, 15 aa, Artificial Sequence
  Conserved peptides
DQETTGFAWWAGNAR

SEQ ID NO: 347 - protein, 9 aa, Artificial Sequence
  Conserved peptides
YPIYVGGNR

SEQ ID NO: 348 - protein, 8 aa, Artificial Sequence
  Conserved peptides
VYDWFEER

SEQ ID NO: 349 - protein, 13 aa, Artificial Sequence
  Conserved peptides
  BINDING (8)..(9): 2-(2-Pyridyl)ethylamine (Pye)
DFGYSFPCDGPGR

SEQ ID NO: 350 - protein, 12 aa, Artificial Sequence
  Conserved peptides
DKPVALSIVQAR

SEQ ID NO: 351 - protein, 18 aa, Artificial Sequence
  Conserved peptides
QILIEPIFAQWIQSAHGK

SEQ ID NO: 352 - protein, 13 aa, Artificial Sequence
  Conserved peptides
VFPNGEVQYLHPK

SEQ ID NO: 353 - protein, 14 aa, Artificial Sequence
  Conserved peptides
FVQAGSEVSALLGR

SEQ ID NO: 354 - protein, 9 aa, Artificial Sequence
  Conserved peptides
LSIFETGIK

SEQ ID NO: 355 - protein, 9 aa, Artificial Sequence
  Conserved peptides
DTDILAAFR

SEQ ID NO: 356 - protein, 13 aa, Artificial Sequence
  Conserved peptides
TFQGPPHGIQVER

SEQ ID NO: 357 - protein, 7 aa, Artificial Sequence
  Conserved peptides
FYWAPTR

SEQ ID NO: 358 - protein, 7 aa, Artificial Sequence
  Conserved peptides
VYDDEVR

SEQ ID NO: 359 - protein, 10 aa, Artificial Sequence
  Conserved peptides
IGVIESLLEK

SEQ ID NO: 360 - protein, 15 aa, Artificial Sequence
  Conserved peptides
AAALNIVPTSTGAAK

SEQ ID NO: 361 - protein, 8 aa, Artificial Sequence
  Conserved peptides
VIITAPAK

SEQ ID NO: 362 - protein, 16 aa, Artificial Sequence
  Conserved peptides
GKRLASIGLENTEANR

SEQ ID NO: 363 - protein, 11 aa, Artificial Sequence
  Conserved peptides
YIGSLVGDFHR

SEQ ID NO: 364 - protein, 8 aa, Artificial Sequence
  Conserved peptides
FFQLYVYK

SEQ ID NO: 365 - protein, 9 aa, Artificial Sequence
  Conserved peptides
NFEGLDLGK

SEQ ID NO: 366 - protein, 12 aa, Artificial Sequence
  Conserved peptides
AIPWIFAWTQTR

SEQ ID NO: 367 - protein, 12 aa, Artificial Sequence
  Conserved peptides
AIPWIFSWTQTR

SEQ ID NO: 368 - protein, 9 aa, Artificial Sequence
  Conserved peptides
EFAPSIPEK

SEQ ID NO: 369 - protein, 16 aa, Artificial Sequence
  Conserved peptides
VLVVANPANTNALILK

SEQ ID NO: 370 - protein, 9 aa, Artificial Sequence
  Conserved peptides
AGLQFPVGR

SEQ ID NO: 371 - protein, 8 aa, Artificial Sequence
  Conserved peptides
IFLENVIR

SEQ ID NO: 372 - protein, 15 aa, Artificial Sequence
  Conserved peptides
VTGGEVGAASSLAPK

SEQ ID NO: 373 - protein, 11 aa, Artificial Sequence
  Conserved peptides
VSGVSLLALFK

SEQ ID NO: 374 - protein, 13 aa, Artificial Sequence
  Conserved peptides
ELAEDGYSGVEVR

SEQ ID NO: 375 - protein, 14 aa, Artificial Sequence
  Conserved peptides
GLDVIQQAQSGTGK

SEQ ID NO: 376 - protein, 10 aa, Artificial Sequence
  Conserved peptides
VLITTDLLAR

SEQ ID NO: 377 - protein, 11 aa, Artificial Sequence
  Conserved peptides
IGGIGTVPVGR

SEQ ID NO: 378 - protein, 8 aa, Artificial Sequence
  Conserved peptides
LPLQDVYK

SEQ ID NO: 379 - protein, 14 aa, Artificial Sequence
  Conserved peptides
GSGFVAVEIPFTPR

SEQ ID NO: 380 - protein, 10 aa, Artificial Sequence
  Conserved peptides
TAIAEGLAQR

SEQ ID NO: 381 - protein, 14 aa, Artificial Sequence
  Conserved peptides
GILAADESTGTIGK

SEQ ID NO: 382 - protein, 10 aa, Artificial Sequence
  Conserved peptides
AVDSLVPIGR

SEQ ID NO: 383 - protein, 14 aa, Artificial Sequence
  Conserved peptides
AHGGFSVFAGVGER

SEQ ID NO: 384 - protein, 10 aa, Artificial Sequence
  Conserved peptides
VVDLLAPYQR

SEQ ID NO: 385 - protein, 10 aa, Artificial Sequence
  Conserved peptides
AGFAGDDAPR

SEQ ID NO: 386 - protein, 11 aa, Artificial Sequence
  Conserved peptides
IWHHTFYNELR

SEQ ID NO: 387 - protein, 16 aa, Artificial Sequence
  Conserved peptides
ATAGDTHLGGEDFDNR

SEQ ID NO: 388 - protein, 16 aa, Artificial Sequence
  Conserved peptides
IINEPTAAAIAYGLDK

SEQ ID NO: 389 - protein, 8 aa, Artificial Sequence
  Conserved peptides
ETDGYFIK

SEQ ID NO: 390 - protein, 14 aa, Artificial Sequence
  Conserved peptides
IYVLTQFNSASLNR

SEQ ID NO: 391 - protein, 6 aa, Artificial Sequence
  Conserved peptides
YNQLLR

SEQ ID NO: 392 - protein, 11 aa, Artificial Sequence
  Conserved peptides
LFTGHPETLEK

SEQ ID NO: 393 - protein, 15 aa, Artificial Sequence
  Conserved peptides
VEADIAGHGQEVLIR

SEQ ID NO: 394 - protein, 10 aa, Artificial Sequence
  Conserved peptides
DEDTQAMPFR

SEQ ID NO: 395 - protein, 16 aa, Artificial Sequence
  Conserved peptides
GGLEPINFQTAADQAR

SEQ ID NO: 396 - protein, 17 aa, Artificial Sequence
  Conserved peptides
ISQAVHAAHAEINEAGR

SEQ ID NO: 397 - protein, 17 aa, Artificial Sequence
  Conserved peptides
WAMLGALGCVFPELLAR

SEQ ID NO: 398 - protein, 14 aa, Artificial Sequence
  Conserved peptides
STPQSIWYGPDRPK

SEQ ID NO: 399 - protein, 8 aa, Artificial Sequence
  Conserved peptides
ALEVIHGR

SEQ ID NO: 400 - protein, 8 aa, Artificial Sequence
  Conserved peptides
ECELIHGR

SEQ ID NO: 401 - protein, 14 aa, Artificial Sequence
  Conserved peptides
LHPGGPFDPLGLAK

SEQ ID NO: 402 - protein, 16 aa, Artificial Sequence
  Conserved peptides
TGALLLDGNTLNYFGK

SEQ ID NO: 403 - protein, 8 aa, Artificial Sequence
  Conserved peptides
EAELIHGR

SEQ ID NO: 404 - protein, 17 aa, Artificial Sequence
  Conserved peptides
GGSTGYDNAVALPAGGR

SEQ ID NO: 405 - protein, 8 aa, Artificial Sequence
  Conserved peptides
GSSFLDPK

SEQ ID NO: 406 - protein, 13 aa, Artificial Sequence
  Conserved peptides
AYGEAANVFGKPK

SEQ ID NO: 407 - protein, 10 aa, Artificial Sequence
  Conserved peptides
AWPYVQNDLR

SEQ ID NO: 408 - protein, 8 aa, Artificial Sequence
  Conserved peptides
ANELFVGR

SEQ ID NO: 409 - protein, 8 aa, Artificial Sequence
  Conserved peptides
ESELIHCR

SEQ ID NO: 410 - protein, 8 aa, Artificial Sequence
  Conserved peptides
QYFLGLEK

SEQ ID NO: 411 - protein, 12 aa, Artificial Sequence
  Conserved peptides
EIPLPHEFILNR

SEQ ID NO: 412 - protein, 8 aa, Artificial Sequence
  Conserved peptides
TAVNPLLR

SEQ ID NO: 413 - protein, 9 aa, Artificial Sequence
  Conserved peptides
VYLWHETTR

SEQ ID NO: 414 - protein, 11 aa, Artificial Sequence
  Conserved peptides
EIIIDVPLASR

SEQ ID NO: 415 - protein, 16 aa, Artificial Sequence
  Conserved peptides
LYSIASSAIGDFGDSK

SEQ ID NO: 416 - protein, 13 aa, Artificial Sequence
  Conserved peptides
GYISPYFVTDSEK

SEQ ID NO: 417 - protein, 12 aa, Artificial Sequence
  Conserved peptides
LADLVGVTLGPK

SEQ ID NO: 418 - protein, 8 aa, Artificial Sequence
  Conserved peptides
AMHAVIDR

SEQ ID NO: 419 - protein, 9 aa, Artificial Sequence
  Conserved peptides
SQAETGEIK

SEQ ID NO: 420 - protein, 17 aa, Artificial Sequence
  Conserved peptides
LDELIYVESHLSNLSTK

SEQ ID NO: 421 - protein, 22 aa, Artificial Sequence
  Conserved peptides
QYADAVIEVLPTTLIPDDNEGK

SEQ ID NO: 422 - protein, 17 aa, Artificial Sequence
  Conserved peptides
GVTTIIGGGDSVAAVEK

SEQ ID NO: 423 - protein, 14 aa, Artificial Sequence
  Conserved peptides
GGAFTGEISVEQLK

SEQ ID NO: 424 - protein, 8 aa, Artificial Sequence
  Conserved peptides
EAAWGLAR

SEQ ID NO: 425 - protein, 12 aa, Artificial Sequence
  Conserved peptides
VTTTIGYGSPNK

SEQ ID NO: 426 - protein, 15 aa, Artificial Sequence
  Conserved peptides
YTGGMVPDVNQIIVK

SEQ ID NO: 427 - protein, 19 aa, Artificial Sequence
  Conserved peptides
IDLAIDGADEVDPNLDLVK

SEQ ID NO: 428 - protein, 10 aa, Artificial Sequence
  Conserved peptides
LVFVTNNSTK

SEQ ID NO: 429 - protein, 18 aa, Artificial Sequence
  Conserved peptides
LLEATGISTVPGSGFGQK

SEQ ID NO: 430 - protein, 9 aa, Artificial Sequence
  Conserved peptides
LAVEAWGLK

SEQ ID NO: 431 - protein, 11 aa, Artificial Sequence
  Conserved peptides
IAILNANYMAK

SEQ ID NO: 432 - protein, 19 aa, Artificial Sequence
  Conserved peptides
SLLALQGPLAAPVLQHLTK

SEQ ID NO: 433 - protein, 9 aa, Artificial Sequence
  Conserved peptides
YSEGYPGAR

SEQ ID NO: 434 - protein, 11 aa, Artificial Sequence
  Conserved peptides
GQTVGVIGAGR

SEQ ID NO: 435 - protein, 10 aa, Artificial Sequence
  Conserved peptides
FDFDPLDVTK

SEQ ID NO: 436 - protein, 8 aa, Artificial Sequence
  Conserved peptides
FSVSPVVR

SEQ ID NO: 437 - protein, 9 aa, Artificial Sequence
  Conserved peptides
GVQYLNEIK

SEQ ID NO: 438 - protein, 15 aa, Artificial Sequence
  Conserved peptides
AASFNIIPSSTGAAK

SEQ ID NO: 439 - protein, 14 aa, Artificial Sequence
  Conserved peptides
VPTVDVSVVDLTVR

SEQ ID NO: 440 - protein, 17 aa, Artificial Sequence
  Conserved peptides
LVAGLPEGGVLLLENVR

SEQ ID NO: 441 - protein, 12 aa, Artificial Sequence
  Conserved peptides
LAADTPLLTGQR

SEQ ID NO: 442 - protein, 15 aa, Artificial Sequence
  Conserved peptides
AVVQVFEGTSGIDNK

SEQ ID NO: 443 - protein, 8 aa, Artificial Sequence
  Conserved peptides
AILNLSLR

SEQ ID NO: 444 - protein, 12 aa, Artificial Sequence
  Conserved peptides
EHIAAYGEGNER

SEQ ID NO: 445 - protein, 16 aa, Artificial Sequence
  Conserved peptides
LVAEAGIGTVASGVAK

SEQ ID NO: 446 - protein, 18 aa, Artificial Sequence
  Conserved peptides
VCPSHILNFQPGEAFVVR

SEQ ID NO: 447 - protein, 9 aa, Artificial Sequence
  Conserved peptides
DVATILHWK

SEQ ID NO: 448 - protein, 10 aa, Artificial Sequence
  Conserved peptides
FALESFWDGK

SEQ ID NO: 449 - protein, 10 aa, Artificial Sequence
  Conserved peptides
DEDTQAMPFR

SEQ ID NO: 450 - protein, 16 aa, Artificial Sequence
  Conserved peptides
GGLEPINFQTAADQAR

SEQ ID NO: 451 - protein, 15 aa, Artificial Sequence
  Conserved peptides
VEADIAGHGQEVLIR

SEQ ID NO: 452 - protein, 684 aa, Artificial Sequence
  Conserved peptides
MAGRNFEGLDLGKELAEDGYSGVEVRAHGGFSVFAGVGERTAIAEGLAQREFAPSIPEKGGLEPINFQTAADQARLPLQDVYKAYDFVSQEIRGKRLASIGLENTEANRDKPVALSIVQARAGFAGDDAPRQILIEPIFAQWIQSAHGKIGGIGTVPVGRVHTVVLNDPGRVYDDEVRLSIFETGIKVYDWFEERLIFQYASFNNSRVSGVSLLALFKETDGYFIKVIITAPAKYPIYVGGNRAVDSLVPIGRAGLQFPVGRVVDLLAPYQRLAFYDYIGNNPAKVLVVANPANTNALILKAIPWIFAWTQTRLFTGHPETLEKFVQAGSEVSALLGRNILLNEGIRFYWAPTRGLDVIQQAQSGTGKATAGDTHLGGEDFDNRDFGYSFPCDGPGRAAALNIVPTSTGAAKISQAVHAAHAEINEAGRYIGSLVGDFHRYNQLLRIGVIESLLEKFFQLYVYKVLITTDLLARIYVLTQFNSASLNRAPWLEPLRGILAADESTGTIGKIWHHTFYNELRVTGGEVGAASSLAPKVFPNGEVQYLHPKVINTWADIINRIFLENVIRIINEPTAAAIAYGLDKTFQGPPHGIQVERGSGFVAVEIPFTPRDQETTGFAWWAGNARVEADIAGHGQEVLIRAIPWIFSWTQTRDTDILAAFRDEDTQAMPFRLAAALEHHHHHH

SEQ ID NO: 453 - protein, 697 aa, Artificial Sequence
  Conserved peptides
HMAGRGGLEPINFQTAADQARLHPGGPFDPLGLAKTGALLLDGNTLNYFGKDEDTQAMPFRWAMLGALGCVFPELLARAWPYVQNDLRYSEGYPGARFSVSPVVRGVQYLNEIKEAELIHGRECELIHGRAYGEAANVFGKPKANELFVGRLVFVTNNSTKLLEATGISTVPGSGFGQKLAVEAWGLKQYFLGLEKESELIHCREIIIDVPLASRVYLWHETTREIPLPHEFILNRTAVNPLLRSTPQSIWYGPDRPKAILNLSLRIAILNANYMAKSLLALQGPLAAPVLQHLTKGQTVGVIGAGRAMHAVIDREHIAAYGEGNERALEVIHGRGVTTIIGGGDSVAAVEKGGAFTGEISVEQLKEAAWGLARGGSTGYDNAVALPAGGRFALESFWDGKFDFDPLDVTKLYSIASSAIGDFGDSKGSSFLDPKLVAEAGIGTVASGVAKSQAETGEIKIDLAIDGADEVDPNLDLVKLDELIYVESHLSNLSTKQYADAVIEVLPTTLIPDDNEGKLADLVGVTLGPKGYISPYFVTDSEKYTGGMVPDVNQIIVKVTTTIGYGSPNKAVVQVFEGTSGIDNKLAADTPLLTGQRLVAGLPEGGVLLLENVRVPTVDVSVVDLTVRAASFNIIPSSTGAAKDVATILHWKVCPSHILNFQPGEAFVVRVEADIAGHGQEVLIRLAAALEHHHHHH

SEQ ID NO: 454 - protein, 16 aa, Artificial Sequence
  Conserved peptides produced by trypsin
AHGGVSVFGGVGERTR

SEQ ID NO: 455 - protein, 15 aa, Artificial Sequence
  Conserved peptides produced by trypsin
VALVYGQMNEPPGAR

SEQ ID NO: 456 - protein, 13 aa, Artificial Sequence
  Conserved peptides produced by trypsin
TVLIMELINNIAK

SEQ ID NO: 457 - protein, 20 aa, Artificial Sequence
  Conserved peptides produced by Glu C
LINNIAKAHGGVSVFGGVGE

SEQ ID NO: 458 - protein, 17 aa, Artificial Sequence
  Conserved peptides produced by Glu C
PPGARMRVGLTALTMAE

SEQ ID NO: 459 - protein, 14 aa, Artificial Sequence
  Conserved peptides produced by Asp N
DTKLSIFETGIKVV

SEQ ID NO: 460 - protein, 11 aa, Artificial Sequence
  Conserved peptides produced by Asp N
DPAPATTFAHL

SEQ ID NO: 461 - protein, 14 aa, Artificial Sequence
  Conserved peptides produced by formic acid cleavage
TKLSIFETGIKVVD

SEQ ID NO: 462 - protein, 11 aa, Artificial Sequence
  Conserved peptides produced by formic acid cleavage
PAPATTFAHLD

SEQ ID NO: 463 - protein, 8 aa, Artificial Sequence
  Conserved peptides produced by cyanogen bromide cleavage
NEPPGARM

SEQ ID NO: 464 - protein, 14 aa, Artificial Sequence
  Conserved peptides produced by cyanogen bromide cleavage
PSAVGYQPTLSTEM

SEQ ID NO: 465 - protein, 9 aa, Artificial Sequence
  Conserved peptides produced by cyanogen bromide cleavage
RVGLTALTM